(19) United States
(12) Patent Application Publication
(10) Pub. No.: US 2023/0167155 A1
(43) Pub. Date: Jun. 1, 2023

(54) COMPOSITIONS AND METHODS

(71) Applicant: KING'S COLLEGE LONDON, London (GB)

(72) Inventors: Anthony DORLING, London (GB); Richard A. SMITH, London (GB)

(21) Appl. No.: 17/904,967

(22) PCT Filed: Feb. 25, 2021
(86) PCT No.: PCT/GB2021/050485

§ 371 Date: Aug. 25, 2022

(30) Foreign Application Priority Data
Feb. 26, 2020 (GB) 2002707.4

(51) Int. Cl. C07K 7/06; A61P 9/10; A61P 13/12

(52) U.S. Cl. CPC C07K 7/06; A61P 9/10; A61P 13/12; A61K 38/00

(57) ABSTRACT

The invention relates to a compound comprising: CH₂—CH₂—F-Cha-Cha-RKPNDK-NH₂ joined via a linking group to [Myr2]-KSSKSPSKKDDKKPGD. The invention also relates to its use in treatment of atheroma, use in treatment of atherosclerosis, use in inducing regression of atherosclerosis, and use in treatment of Acute Kidney Injury (AKI).

### FIELD OF THE INVENTION

The invention is in the field of atherosclerosis and associated disorders and treatment of same.

### BACKGROUND

Atherosclerosis is a chronic inflammatory disease that causes coronary artery, peripheral vascular and cerebrovascular disease. It is a major cause of death in the Western world. Aside from dietary manipulation and statins to reduce plasma lipids, there are no other proven treatments to reverse (or regress) the disease process.

Important early steps in atherogenesis, such as in the context of a high lipid microenvironment, include secretion of chemokines such as CCL-2 and macrophage migration inhibitory factor (MIF), by activated endothelial cells (ECs) and smooth muscle cells (SMCs). These promote infiltration of monocytes into the subendothelial space, where they become macrophages and take up very low-density lipoprotein (VLDL) and low-density lipoprotein (LDL) to become foam cells, initiating the process of atheroma formation.

Continuous monocyte recruitment into the vessel wall is one of the major steps in the pathogenesis of atherosclerosis, as evidenced by studies showing that simultaneous inhibition of the chemokines CCL2, CX3CR1 and CCR5 (all involved in monocyte recruitment), near abolishes development of atheroma in ApoE−/− mice. In addition, deficiency of MIF also impairs atheroma development in LDL-R deficient mice and an inhibitory anti-MIF antibody has been shown to prevent atherosclerosis in ApoE−/− mice. The foam cells which develop from the recruited monocytes have a distinct proinflammatory phenotype and eventually undergo cell death, perpetuating an inflammatory cycle of recruitment and plaque expansion.

Studies in which atheroma regression has been induced, many of which have involved transplantation of atheromatous aorta from ApoE−/− mice into BL/6 mice, identify the monocyte/macrophage phenotype as the critical determinant of regression. Therefore, maintenance of expression of the chemokine receptor CCR7 by foam cells allowed them to leave the atherosclerotic plaque (emigrate), as demonstrated by inhibiting the chemokine ligands for CCR7. In another model, LDLR−/− mice treated with an antisense to miR-33 showed regression associated with upregulated ABCA1 expression in plaque macrophages and enhanced reverse cholesterol transport, in association with increased levels of circulating HDL, consistent with the known importance of ABCA1 for cholesterol loading into HDL and with the phenotype of ABCA1-deficient mice. In addition, the importance of polarising new monocyte recruits to the plaque towards an M2 phenotype has been recently demonstrated in the aortic transplant model, by confirming that regression is dependent on the expression of both appropriate chemokine receptors (CCR2/CX3CR1) and the transcription factor STAT6 by recipient monocytes.

Coagulation proteases, such as thrombin, signal though protease activated receptors (PAR) as well as catalysing fibrin formation and are known to play a role in atheroma formation and progression.

Key evidence for this is as follows;
Increased activity of tissue factor (TF), the 47-Kd cell membrane-bound glycoprotein that initiates the serine protease cascade, is seen in the neointima and underlying media of atherosclerotic plaques and TF is expressed by EC, monocytes/macrophages and SMC.
ApoE−/− mice made deficient in HCII, a natural thrombin inhibitor, or carrying a DNA variant resulting in defective thrombomodulin-mediated generation of activated protein C develop severe atheroma, indicating that in this model, endogenous regulators of thrombin act to limit disease severity.
Transgenic expression of a TF inhibitor, TFPI, on SMC of ApoE−/− mice completely inhibited development of atheroma in mice fed a high fat diet, via suppression of the chemokine gradients involved in recruitment of monocytes.
Factor (F)Xa inhibitors and direct thrombin inhibitors prevent atheroma progression and maintain plaque stability.
Systemic anticoagulants can also induce regression of atherosclerosis in ApoE−/− mice. Megalatran used in 30-week-old animals showed reduced burden of advanced atheromatous lesions associated with plaque stability. Atheroma burden is reduced in 22 week old animals by 25% after daily treatment for 6 weeks with clinically relevant doses of the FXa inhibitor rivaroxaban.

However, clinical translation of these effects suffers from the drawback of a significant increase in the incidence of major bleeding events. This causes serious risk of death from events such as brain haemorrhage or other internal bleeds, as well as the range of risks associated with impaired clotting. This is a very serious problem with using systemic anticoagulants or antiplatelet drugs for non-thrombotic diseases. It is a problem that the negative impact on haemostasis cannot be separated from the clinical efficacy for known compounds in this field.

Kawabata 1999 (J Pharm and Exp Ther volume 288 pages 358-370) discloses evaluation of proteinase activated receptor 1 (PAR-1) agonists and antagonists using a cultured cell receptor desensitisation assay: activation of PAR-2 by PAR-1 targeted ligands. This disclosure does not relate to delivering any PAR-1 antagonist/PAR-2 agonist as being clinically useful. This disclosure is confined to the particular cultured cell assay developed for in vitro use in assessing the selectivity of PAR-activating agonists in the laboratory. This disclosure does not relate to atheroma, atherosclerosis or regression of atherosclerotic plaques in any way.

WO/2011/027175 discloses a soluble compound for preventing or reducing blood coagulation comprising an antithrombotic agent and a membrane binding element. This document neither discloses, nor suggests, the compounds disclosed herein.

The present invention seeks to overcome problem(s) associated with the art.

### SUMMARY

The invention is based on novel compounds (exemplified with compounds PTL032 and PTL0GC-1) that have significant unprecedented potency at inducing regression of atherosclerosis. The inventors believe that amongst the reasons for this very surprising activity is the dual targeting of PAR-1 and PAR-2 signalling in a single molecule. The inventors also believe that the new compounds offer a level of safety not offered by other agents targeting these pathways, and that amongst the reasons for this is the cytotopic tail that uncouples the potent effect on inflammation from that on haemostasis.

The inventors support their breakthrough finding with comparative data which directly demonstrate the technical advances provided by the invention. The invention is based on these surprising advances.

Thus in one aspect the invention relates to a compound comprising:

### CH2-CH2-F-Cha-Cha-RKPNDK-NH2

joined via a linking group to

### [Myr2]-KSSKSPSKKDDKKPGD.

In another embodiment the invention relates to a compound as described above which has the formula:

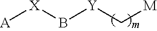

wherein

### A is CH2-CH2-F-Cha-Cha-RKPNDK-NH2

### M is [Myr2]-KSSKSPSKKDDKKPGD

### X is S, O, or NR

B is optional and is an optionally substituted C1 to C6 alkyl

### Y is S, O, or NR

m is 1, 2, 3, 4, 5, or 6
wherein each R is independently selected from H, or optionally substituted C1-6 alkyl

In one embodiment suitably said linking group comprises a disulphide bridge. In one embodiment suitably said linking group consists of a cysteine residue-disulphide bridge.

In one embodiment suitably said linking group comprises a thioether group. In one embodiment suitably said linking group consists of a lysine residue-thioether group.

In one embodiment suitably the compound has the formula:

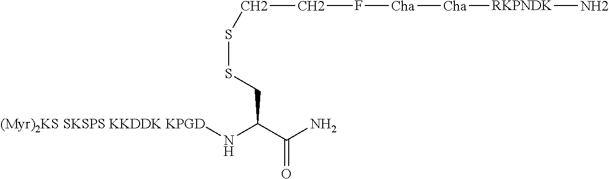

In one embodiment suitably the compound has the formula:

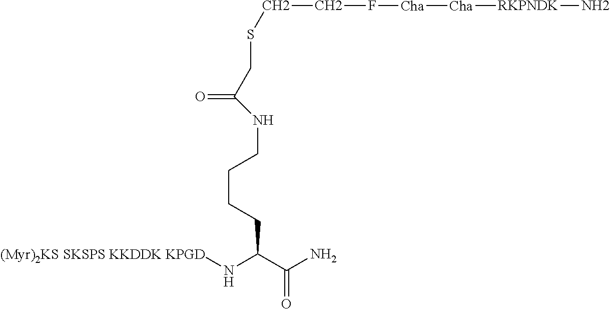

In another embodiment the invention relates to a compound as described above for use in medicine.

In another embodiment the invention relates to a compound as described above for use as a medicament.

In another embodiment the invention relates to use of a compound as described above for the manufacture of a medicament for atheroma.

In another embodiment the invention relates to use of a compound as described above for the manufacture of a medicament for atherosclerosis.

In another embodiment the invention relates to use of a compound as described above for the manufacture of a medicament for inducing regression of atherosclerosis.

In another embodiment the invention relates to a compound as described above for use in treatment of atheroma.

In another embodiment the invention relates to a compound as described above for use in treatment of atherosclerosis.

In another embodiment the invention relates to a compound as described above for use in inducing regression of atherosclerosis.

In another embodiment the invention relates to a method of treatment comprising administering a therapeutic amount of a compound as described above to a subject in need of same.

In another embodiment the invention relates to a method of treating atheroma in a subject comprising administering a therapeutic amount of a compound as described above to said subject.

In another embodiment the invention relates to a method of treating atherosclerosis in a subject comprising administering a therapeutic amount of a compound as described above to said subject.

In another embodiment the invention relates to a method of inducing regression of atherosclerosis in a subject comprising administering a therapeutic amount of a compound as described above to said subject.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### DETAILED DESCRIPTION

In one aspect the invention relates to a compound having the formula:

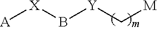

wherein
A is the active part of the compound, most suitably a (PAR2 agonist-PAR1 antagonist) part of the compound, and
M is a cytotopic tail, most suitably a membrane anchor.

In one embodiment suitably the active part of the compound (A), comprises a (PAR2 agonist-PAR1 antagonist). More suitably the active part of the compound (A) comprises CH2-CH2-F-Cha-Cha-RKPNDK-NH2. Most suitably the active part of the compound (A) consists of CH2-CH2-F-Cha-Cha-RKPNDK-NH2.

In one embodiment suitably the cytotopic tail (M), comprises a membrane anchor. More suitably the cytotopic tail (M) comprises [Myr2]-KSSKSPSKKDDKKPGD. Most suitably the cytotopic tail (M) consists of [Myr2]-KSSKSPSKKDDKKPGD.

In one aspect the invention relates to a compound comprising:

### CH2-CH2-F-Cha-Cha-RKPNDK-NH2

joined via a linking group to

### [Myr2]-KSSKSPSKKDDKKPGD.

In one embodiment the linking group comprises a cysteine residue (PTL032).

In one embodiment the linking group comprises a lysine residue (PTL0GC1).

Suitably the compound is a soluble compound. Suitably soluble means soluble in aqueous solvent. Suitably soluble means soluble in water. Suitably soluble means soluble in serum or plasma.

In one aspect the invention relates to a compound having the formula: SEQ ID NO: 1-linker-SEQ ID NO: 2.

In one embodiment the invention relates to a compound having the formula: SEQ ID NO: 1-linker-SEQ ID NO: 3.

In one embodiment the invention relates to a compound having the formula: SEQ ID NO: 1-linker-SEQ ID NO: 4.

The active part (A) is suitably joined at the first C of the first CH2 to the linker. For the avoidance of doubt exemplary structures are disclosed to illustrate the joining.

The cytotopic tail (M) is suitably joined at the last D of the . . . PGD to the linker. For the avoidance of doubt exemplary structures are disclosed to illustrate the joining.

### Linking Group

Sometimes the linking group may be referred to as the linker. This means the atoms between the active part of the compound (A) and the cytotopic tail (M).

Suitably the linking group is as below:

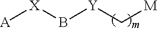

wherein
A is the active part of the compound, most suitably the (PAR2 agonist-PAR1 antagonist) part of the compound;
M is the cytotopic tail, most suitably the membrane anchor part of the compound; and the intervening atoms are the linking group (linker):

### X is S, O, or NR

B is optional and is an optionally substituted C1 to C6 alkyl

### Y is S, O, or NR

m is 1, 2, 3, 4, 5, or 6
wherein each R is independently selected from H, or optionally substituted C1-6 alkyl

As will be apparent to the skilled reader, the stereochemistry (i.e. bond orientation/bond geometry) is not indicated for the bond from M to first member of linking group. In one embodiment suitably the direction of bond is out of the page from M to first member of linking group.

“Substituted”, when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

“Optionally substituted” refers to a parent group which may be unsubstituted or which may be substituted with one or more substituents. Suitably, unless otherwise specified, when optional substituents are present the optional substituted parent group comprises from one to three optional substituents.

Substituents may be selected from C₁₋₆ alkyl, C₂₋₇ alkenyl, C₂₋₇ alkynyl, C₁₋₆ alkoxy, C₅₋₂₀ aryl, C₃₋₁₀ cycloalkyl, C₃₋₁₀ cycloalkenyl, C₃₋₁₀ cycloalkynyl, C₃₋₂₀ heterocyclyl, C₃₋₂₀ heteroaryl, acetal, acyl, acylamido, acyloxy, amidino, amido, amino, aminocarbonyloxy, azido, carboxy, cyano, ether, formyl, guanidino, halo, hemiacetal, hemiketal, hydroxamic acid, hydroxyl, imidic acid, imino, ketal, nitro, nitroso, oxo, oxycarbonyl, oxycarboyloxy, sulfamino, sulfamyl, sulfate, sulfhydryl, sulfinamino, sulfinate, sulfino, sulfinyl, sulfinyloxy, sulfo, sulfonamido, sulfonamino, sulfonate, sulfonyl, sulfonyloxy, uredio groups.

For example in one embodiment when the compound is PTL032 the linking group comprises a disulphide bridge.

In one embodiment, when the compound is PTL032 suitably the linking group has the formula above wherein:

### X is S

B is absent

### Y is S

m is 1

For example in one embodiment when the compound is PTL0GC-1 the linking group comprises a thioether group.

In one embodiment, when the compound is PTL0GC-1 suitably the linking group has the formula above wherein:

### X is S

B is substituted C2 alkyl, specifically —CH2C(═O)—

### Y is NR, R is H

m is 4

### PTL032

In one embodiment the invention relates to compound:

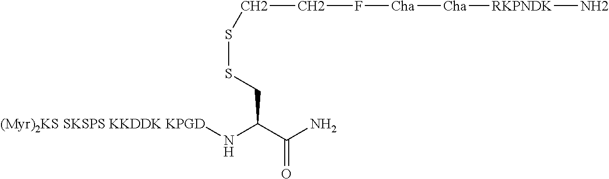

In one embodiment the invention relates to a compound:

### [Myr2]-KSSKSPSKKDDKKPGDC(S—S—CH2-CH2-Phe-Cha-Cha-Arg-Lys-Pro-Asn-Asp-Lys-NH2)NH2

In the written formula the Cysteine residue is written as ‘C’ ( . . . GDC).

In the drawn structure the Cysteine residue is drawn out in full.

### PTL0GC1

In one embodiment the invention relates to a compound:

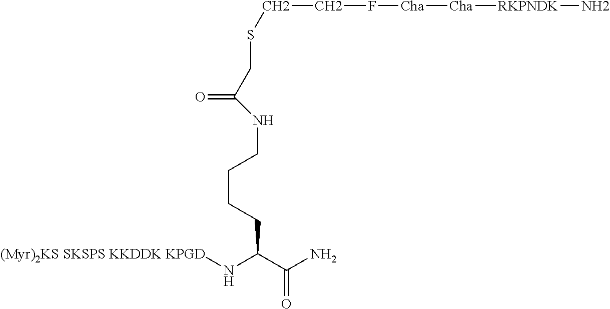

In one embodiment the invention relates to a compound:

### [Myr2]-KSSKSPSKKDDKKPGDK(CO—CH2-S—CH2-CH2-Phe-Cha-Cha-Arg-Lys-Pro-Asn-Asp-Lys-NH2)NH2

In the written formula the Lysine residue is written as ‘K’ ( . . . GDK).

In the drawn structure the Lysine residue is drawn out in full.

### FURTHER DESCRIPTION

The membrane binding element is capable of binding to a cell membrane such as a mammalian cell membrane. Suitable naturally-occurring membrane binding elements are well known to those skilled in the art, either as components of proteins that mediate membrane interactions or as membrane components such as sterols or sphingolipids.

The membrane binding element can bind to the membrane surface of a cell so as to localise the antithrombotic agent upon the external surface of the cell in a manner that permits the functionality of the antithrombotic agent to be expressed against its target. The membrane binding element should be sufficiently hydrophilic so that the compound of the invention is soluble.

The membrane binding element may optionally or additionally comprise: fatty acid derivatives such as fatty acyl groups; basic amino acid sequences; ligands of known integral membrane proteins; sequences derived from the complementarity-determining region of monoclonal antibodies raised against epitopes of membrane proteins; and membrane binding sequences identified through screening of random chemical or peptide libraries. In one embodiment, the membrane binding element may optionally or additionally be a phospholipid which has been derivatised to increase its water-solubility. For example, the phospholipid may optionally or additionally be derivatised with a hydrophilic polymer, such as polyethylene glycol (PEG), polyvinylpyrrolidone, dextran, or polysarcosine. Other suitable polymers are apparent to a skilled person. The membrane binding element may optionally or additionally comprise a glycosylphosphatidylinositol (GPI) anchor or an analogue thereof. Suitable GPI anchors and analogues are well known to those skilled in the art and are described, for example, in Paulick M G and Bertozzi C R (Biochemistry 47: 6991-7000, 2008). The carbohydrate portion of the GPI anchor may be comprised of any suitable saccharide monomers. Suitable saccharide monomers will be apparent to one skilled in the art as will the length of the carbohydrate portion.

In an alternative embodiment, the membrane binding element may be a peptide which is capable of interacting with one or more components of the outer cell membranes of cells, for example, phospholipids. Preferably, the peptide is between 3 and 25 amino acids. More preferably, the peptide is between 4 and 20 amino acids. Preferably, the peptide is a hydrophilic peptide. In one embodiment, the peptide comprises between three and 8 lysine residues, preferably, L-lysine residues.

The peptide may additionally comprise one or more groups which are capable of interacting with the lipid bilayer core of a cell membrane. Suitable groups are well known to those skilled in the art. These groups should be hydrophobic groups. For example, the one or more groups may be fatty acyl groups, such as myristoyl and/or palmitoyl groups. Preferably, the one or more groups are located at or near the N-terminal of the peptide. Other examples of suitable hydrophobic groups include long-chain aliphatic amines and thiols, steroid and farnesyl derivatives. This approach is based on the structure and function of the myristoyl-electrostatic switch (MES) (Thelen M et al. Nature 351: 320-2, 1991). In one embodiment, the one or more group is an isoprenoid group such as farnesyl and geranylgeranyl residues. The membrane binding element may be a plurality of groups which are capable of interacting with the lipid bilayer core of a cell membrane.

In another embodiment, the membrane binding element may be one or more groups which are capable of interacting with the lipid bilayer core of a cell membrane. These groups should be hydrophobic groups. The one or more groups may be fatty acyl groups, such as myristoyl, palmitoyl, or stearoyl groups. Other examples of suitable hydrophobic groups include long-chain aliphatic amines and thiols, steroids and farnesyl derivatives. In one embodiment, the one or more group is an isoprenoid group such as farnesyl and geranylgeranyl residues. The membrane binding element may be a plurality of groups which are capable of interacting with the lipid bilayer core of a cell membrane.

The compound of the invention may comprise one or more membrane binding elements. Suitably the compound comprises one membrane binding element.

Most suitably the compound comprises one membrane binding element.

Most suitably the membrane binding element comprises, or consists of:

### [Myr2]-KSSKSPSKKDDKKPGD

### USES AND APPLICATIONS

The invention finds application in treatment or prevention of atherosclerosis.

The invention finds application in reversal or regression of atherosclerosis.

The invention finds application in inducing regression of atherosclerotic plaques.

The invention finds application in preventing or inhibiting formation of atherosclerotic plaques.

The invention finds application in treatment or prevention of inflammatory disease such as chronic inflammatory disease.

The invention finds application in treatment or prevention of coronary artery disease.

The invention finds application in treatment or prevention of peripheral vascular disease.

The invention finds application in treatment or prevention of cerebrovascular disease.

Compounds of the invention find application in regression of atherosclerosis.

Compounds of the invention find application in regression of atherosclerotic plaques.

Compounds of the invention find application in manufacture of a medicament for regression of atherosclerotic plaques.

In another embodiment the invention relates to delivering a PAR-1 antagonist/PAR-2 agonist as useful for regression of atherosclerotic plaques.

In another embodiment the invention relates to use of a (PAR-1 antagonist/PAR-2 agonist) for treatment of atherosclerotic plaques. Suitably said treatment is regression of atherosclerotic plaques.

Unless otherwise apparent, the terms “treating” and “regression” of atherosclerosis should have their normal meaning in the art.

It should be noted that “treating” atherosclerosis may include ‘holding it at bay’ so that it does not worsen e.g. preventing and/or arresting the growth or expansion or proliferation of plaque(s). This may include stabilisation of a subject in a condition such that their condition does not decline, and/or may include reduction, regression, shrinking or eliminating plaques since clearly that is also a valuable treatment of a subject.

It should be noted that “regression” of atherosclerosis includes facilitating or inducing reduction of plaques such as reduction in number or reduction in size or reduction in severity; this may include the effect that a plaque is visibly lessened upon imaging. This may be referred to as ‘reversal’ or ‘reversing’. Thus regression is an especially advantageous treatment.

The compound of the invention can bind to the cell membrane of cells, tissues and organs to prevent or reduce the formation of blood clots. Since the compound can bind to cell membranes, it can be administered locally so that the compound has an effect at a specific location rather than having a systemic effect. The compound can be used in the short-term manipulation of organs in transplantation. It can also be used in cell therapies in which it is desirable to confer resistance to coagulation.

An advantage provided by the compound is that it is relatively small and so it is relatively easy to manufacture. Further, the compound can be manufactured synthetically.

In another embodiment the invention relates to use of a compound as described above, or an aqueous solution of a compound as described above, for perfusing or bathing organ(s), tissue(s) or cell(s), more suitably for perfusing or bathing mammalian organ(s), tissue(s) or cell(s). In one embodiment said perfusion is in vitro. In one embodiment said perfusion is in vivo.

In one embodiment the present invention provides a method of perfusing an organ, tissue or cell, more suitably for perfusing or bathing mammalian organ(s), tissue(s) or cell(s), comprising contacting a compound as described above with the organ, tissue or cell. Suitably said method is carried out to prevent or reduce blood coagulation.

In one embodiment the present invention provides a method of perfusing an organ comprising contacting a compound as described above with the blood vessel(s) of an organ so that the compound binds to the blood vessel(s) of the organ in order to prevent or reduce blood coagulation in the organ, or to prevent or reduce atherosclerotic plaque(s) in the organ.

The method can additionally comprise the step of washing the organ, tissue or cell. Cells can be washed by successive centrifugation and resuspension steps to remove compound not bound to the cell surface. Washing of an organ can be through repeat perfusion with a solution not containing the compound.

The compound can be administered in any suitable form. The compound is suitably in a solution and, more suitably, a physiologically acceptable solution. Suitably the solution is aqueous.

The organ should be perfused for sufficiently long to ensure transfer of the compound to the vascular surface of the organ.

The cells that are perfused can be any suitable cells. In certain embodiments, the cells are derived from human blood, human embryonic or induced pluripotent stem cells, and may include erythrocytes, platelets, lymphocytes, fibroblasts, mesenchymal stem cells and endothelial, epithelial or stromal cells.

The organ can be any suitable organ, for example, the heart, liver, kidney, lungs, pancreas including pancreas islets, skin or corneum.

Additionally, the present invention provides an organ, tissue or cell which has been perfused with a compound as described above.

### Formulation

The invention also relates to compositions comprising one or more of the compounds disclosed herein. The composition may be a pharmaceutical composition.

In another embodiment the invention relates to a pharmaceutical composition comprising a compound as described above and one or more pharmaceutically acceptable excipients.

The invention also relates to a pharmaceutical composition comprising the compound described above and one or more pharmaceutically acceptable excipients. Suitable pharmaceutical excipients are well known to those skilled in the art. Pharmaceutically acceptable excipients that may be used in the pharmaceutical composition of this invention include, but are not limited to serum proteins, such as human serum albumin, buffer substances such as phosphates, glycerine, sorbic acid, potassium sorbate, partial glyceride mixtures of saturated vegetable fatty acids, water, salts or electrolytes, disodium hydrogen phosphate, potassium hydrogen phosphate, sodium chloride.

When the pharmaceutical compositions of this invention are administered to a subject, they may be administered in any suitable way. Preferably, the composition is administered by injection, more preferably by local injection into an organ or a site of disease. The pharmaceutical compositions of this invention may contain any conventional non-toxic pharmaceutically-acceptable carriers or vehicles. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant. It should be noted that if oil-in-water dispersions are employed, the compound of the invention is, by virtue of its hydrophobic components, likely to localise on the surface of the oil droplets. This may affect the rate at which the compounds transfer to the cell surface when the dispersion is contact with cells or the vasculature of an organ. Preferably, the compound is in an aqueous solution.

Other pharmaceutically acceptable additives which may be added to the composition are well known to those skilled in the art.

The composition is suitably sterile.

Provided are ‘unit doses’. A unit doses comprises a syringe containing an amount of the composition of the invention for administration to a subject, such as an amount for administration to an adult human.

The compound of the invention may be formulated in any suitable diluent, excipient or vehicle for administration to mammals, most suitably administration to humans.

Suitably the compound of the invention may be formulated for intravenous administration.

Suitably the compound of the invention may be formulated in sterile saline solution.

Suitably the sterile saline solution is of a comparable osmolarity to human blood.

### Administration

Administration may be by subcutaneous injection.

Administration may be by intravenous injection.

Administration may be by patch e.g. patch applied to the skin.

More suitably administration is by subcutaneous injection.

More suitably administration is by intravenous injection.

Most suitably administration is by intravenous injection.

Administration is suitably once per week.

### Subject

Suitably the subject is a mammal, more suitably a human. Most suitably the subject is an adult human. Adult means 18 years old, or more.

Doses are typically determined by a physician with regard to a subject's weight as well as any other factor(s) considered relevant such as sex, age, condition, and any other clinical observations to be taken into account.

Unless otherwise apparent from the text, doses provided herein are for adult humans.

### Dose

The examples provided include administration to mammals. The examples provided include administration to mice.

Mice typically weigh about 20 gms; humans typically weigh about 60 to 80 kg.

Therefore, humans are about 3000 to 4000 times larger than mice.

Thus suitably mice such as adult mice are considered to weigh 20 g. Suitably adult humans are considered to weigh 60-80 Kg. Therefore amounts of components in the doses/compositions as described may be converted into ‘mg/Kg’ or other units if desired.

Amounts of components in the doses/compositions as described in the examples may be expressed in the same ‘mg/Kg’ terms for comparison. By way of example, a dose comprising 7 μg/g (dose/bodyweight) for administration to a mouse such as an adult mouse equates to a dose of 7 mg/Kg.

By way of example, a dose comprising 7 μg compound of the invention/g bodyweight (i.e. 7 μg/g or 7 mg/Kg) for administration to an adult human equates to a dose of (420 mg/60 Kg to 560 mg/80 Kg).

Suitably the dose for an adult human is 7 mg/Kg.

Suitably the actual amount administered as a dose is determined taking account of the weight of the subject.

In case any further guidance is required, it is common for clinicians to carry out a routine dose finding study, to determine safe/effective doses for human patients. In general, humans require less than mice, so it is likely that the clinical dose for humans is lower than the mg/kg values for mice. This is well understood by those in the field. For example, there are U.S. food and drug administration (FDA) guidelines for Human Equivalent Doses (HED), which are suitably used when determining clinical (human) doses.

For example we refer to Nair and Jacob 2016 (Journal of Basic and Clinical Pharmacy volume 7 pages 27-31) which specifically addresses best practice in this area and provides worked examples of the relevant dose calculations for humans starting from animal model doses.

Thus suitably standard approaches are used to determine the safe/effective human dose, most suitably beginning with calculated HED according to Nair and Jacob 2016 (Journal of Basic and Clinical Pharmacy volume 7 pages 27-31)—this document is incorporated herein by reference specifically for the dose calculations beginning with the animal no observed adverse effect levels (NOAEL) dose and resulting the human equivalent dose (HED). Herein, for the purposes of the calculations the NOAEL dose can be taken from the examples section where the animal is a mouse.

Suitably the human dose further has a safety factor applied as in Nair and Jacob 2016.

Suitably the human dose further has a pharmacologically active dose adjustment as in Nair and Jacob 2016.

### NOTATION/NOMENCLATURE

Standard notation/nomenclature is used unless otherwise apparent from the context. The universal abbreviations for amino acid residues and nucleotide residues are used (‘single letter code’) unless otherwise apparent.

Cha represents cyclohexylalanine.

CH2-CH2-F-Cha-Cha-RKPNDK-NH2 may occasionally be written N-(3-mercaptopropionyl)-F-Cha-Cha-RKPNDK-amide; in more detail N-(3-mercaptopropionyl)-F-Cha-Cha-RKPNDK-amide is suitably used as a component in the manufacture of the compounds of the invention such that when N-(3-mercaptopropionyl)-F-Cha-Cha-RKPNDK-amide is reacted/conjugated to join it to the membrane-binding part of the compound (M) this leaves the residual CH2-CH2-F-Cha-Cha-RKPNDK-NH2 as the active part (A) of the compound of the invention.

[Myr2] represents N-(α,ε bis-myristoyl).

[Myr2K] represents N-(α,ε bis-myristoyl lysine).

[Myr2]-KSSKSPSKKDDKKPGD may occasionally be written N-(α,ε bis-myristoyl lysine) SSKSPSKKDDKKPGD; in more detail N-(α,ε bis-myristoyl lysine) SSKSPSKKDDKKPGD is suitably used as a component in the manufacture of the compounds of the invention such that when N-(α,ε bis-myristoyl lysine) SSKSPSKKDDKKPGD is reacted/conjugated to join it to the active part of the compound (A) this leaves the residual [Myr2]-KSSKSPSKKDDKKPGD as the membrane binding part (M) of the compound of the invention.

As the skilled reader will appreciate, this is a slight simplification in the interests of promoting understanding of the invention—in practice [Myr2]-KSSKSPSKKDDKKPGD will include a final ‘C’ for PTL032 ([Myr2]-KSSKSPSKKDDKKPGDC or N-(α,ε bis-myristoyl lysine) SSKSPSKKDDKKPGDC); and in practice [Myr2]-KSSKSPSKKDDKKPGD will include a final ‘K’ for PTL0GC1 ([Myr2]-KSSKSPSKKDDKKPGDK or N-(α,ε bis-myristoyl lysine) SSKSPSKKDDKKPGDK) where the ‘C’ for PTL032 contributes to the linking group for PTL032 and where the ‘K’ for PTL0GC1 contributes to the linking group for PTL0GC1.

In the unlikely event of any doubt remaining, we rely on the structures drawn out herein.

### FURTHER ASPECTS AND FURTHER APPLICATIONS

Described herein is a compound comprising a PAR1 antagonist joined to a cytotopic tail. Suitably the invention relates to a compound comprising a PAR2 agonist-PAR1 antagonist joined to a cytotopic tail. More suitably the invention relates to a compound consisting of a PAR2 agonist-PAR1 antagonist joined to a cytotopic tail. Suitably the PAR2 agonist-PAR1 antagonist is a single element/single part of the molecule e.g. a single active peptide part of the molecule.

This dual signalling is a key part of the present invention. In one embodiment this dual signalling is induced or mediated via a single element/single part of the molecule e.g. a single active peptide part of the molecule.

Most suitably the PAR2 agonist-PAR1 antagonist comprises, or consists of, CH2-CH2-F-Cha-Cha-RKPNDK-NH2.

In one embodiment suitably the cytotopic tail comprises, or consists of, [Myr2]-KSSKSPSKKDDKKPGDC.

In one embodiment suitably the cytotopic tail comprises, or consists of, [Myr2]-KSSKSPSKKDDKKPGDK.

Most suitably the cytotopic tail comprises, or consists of, [Myr2]-KSSKSPSKKDDKKPGD.

This combination of elements in the same compound is surprisingly effective, as explained in detail herein and supported with data and evidence (including comparative data) below.

In one embodiment the invention finds application as an antithrombotic, such as a membrane bound antithrombotic. Thus in one embodiment the invention relates to use of a compound as described above for the manufacture of a medicament for preventing or reducing blood coagulation. In one embodiment the invention relates to use of a compound as described above for use in treatment or reduction of thrombosis.

In one embodiment the invention relates to a method of treating or reducing thrombosis in a subject comprising administering a therapeutic amount of a compound as described above to said subject.

Further clinical indications or uses of the invention include treatment or prevention of: Acute coronary syndrome—post PCI;

Stable symptomatic angina;
Symptomatic peripheral vascular disease; or

### Familial Hypercholesterolaemia.

Suitably the invention relates to a compound as described above for treatment of Acute coronary syndrome—post PCI, Stable symptomatic angina, Symptomatic peripheral vascular disease or Familial Hypercholesterolaemia.

Suitably the invention relates to use of a compound as described above for manufacture of a medicament for Acute coronary syndrome—post PCI, Stable symptomatic angina, Symptomatic peripheral vascular disease or Familial Hypercholesterolaemia. Suitably the invention relates to a method of treatment of Acute coronary syndrome—post PCI, Stable symptomatic angina, Symptomatic peripheral vascular disease or Familial Hypercholesterolaemia, comprising administering to a subject a therapeutically effective amount of a compound as described above.

As mentioned above, the organ may be kidney. The invention finds application in Acute Kidney Injury (AKI); (sometimes called acute kidney damage). This can arise for a number of reasons including from taking of herbal therapies such as taking an excess amount of certain Chinese herbal therapies; this can also arise from ischaemia reperfusion injury.

Thus in one embodiment the invention relates to a method of treatment or prevention of Acute Kidney Injury (AKI), comprising administering to a subject a therapeutically effective amount of a compound as described above.

Thus in one embodiment the invention relates to use of a compound as described above for the manufacture of a medicament for treatment or prevention of Acute Kidney Injury (AKI).

Thus in one embodiment the invention relates to a compound as described above for use in treatment or prevention of Acute Kidney Injury (AKI).

For Acute Kidney Injury (AKI), most suitably the compound is PTL-GC1.

Acute kidney injury (AKI), resulting in sudden loss of kidney function, is an important cause of morbidity and mortality. However, whereas it used to be thought of as a recoverable injury with no long-term sequelae, it is now recognised that even a single episode of AKI can accelerate progression through to chronic kidney disease (CKD) in some individuals, by initiating the progressive replacement of specialised nephrons and kidney microvasculature by fibrous tissue. Thus, Acute Kidney Injury (AKI) can progress to chronic kidney disease (CKD).

Currently, there is no way of identifying those with AKI at risk of progressing to CKD, and no way of preventing it from occurring. The implication of our data (see Examples section, especially Example 6) is that compounds of the invention such as PTL0GC-1 can break this link between AKI and CKD, that compounds of the invention such as PTL0GC-1 can hasten functional recovery of kidney function and reduce the degree of fibrosis that results following AKI. Importantly, our data was generated in a model of vascular injury (ischaemia reperfusion injury) but it is thought that the cellular and molecular processes at work driving fibrosis in this model are the same in other types of AKI, induced by non-vascular insults (such as tubular toxins). Thus we show that the invention can be expected to have significant therapeutic benefit, particularly if given to individuals at the time of their AKI, or in individuals known to be at high risk of developing AKI (such as those undergoing cardiac or vascular surgery, or those with existing CKD).

Thus in one embodiment the invention relates to a method of treatment or prevention of chronic kidney disease (CKD), comprising administering to a subject a therapeutically effective amount of a compound as described above.

Thus in one embodiment the invention relates to use of a compound as described above for the manufacture of a medicament for treatment or prevention of chronic kidney disease (CKD).

Thus in one embodiment the invention relates to a compound as described above for use in treatment or prevention of chronic kidney disease (CKD).

The invention also finds application in delayed type hypersensitivity (DTH) response (or Type IV hypersensitivity response). This is the archetypal antigen-specific cell mediated immune response involving CD4+ T cells and monocytes/macrophages. It underpins the chronic inflammatory lesions that are characteristic of multiple diseases, including, but not limited to, contact dermatitis, inflammatory bowel disease, chronic infection, sarcoidosis (and other granulomatous diseases), and rejection of transplanted organs. The model used herein is an ear swelling (contact dermatitis) model.

Thus, the inventors expect that

a) PTL060 will inhibit the inflammatory component of these diseases which is expected to have significant therapeutic benefit; and
b) PTL032 and PTL0GC-1 will be more effective than PTL060 in these diseases—i.e. will have greater therapeutic value.

Thus in one embodiment the invention relates to a method of treatment or prevention of delayed type hypersensitivity (DTH), comprising administering to a subject a therapeutically effective amount of a compound as described above.

Thus in one embodiment the invention relates to use of a compound as described above for the manufacture of a medicament for treatment or prevention of delayed type hypersensitivity (DTH).

Thus in one embodiment the invention relates to a compound as described above for use in treatment or prevention of delayed type hypersensitivity (DTH).

Without wishing to be bound by theory, the inventors assert that the molecular basis for the superior effectiveness of the compounds of the invention (such as PTL032/PTL0GC-1) compared to PTL060 can be explained by the unique combination of PAR-1 antagonism and PAR-2 agonism, both of which signal via different intracellular pathways, to

a) promote the differential recruitment of monocytes subsets to sites of inflammation and
b) dampen the sensitivity of monocytes/macrophages to stimulation by interferon gamma.

Data in support of these novel insights are presented in the Examples section.

### FURTHER ADVANTAGES

The inventors believe that PTL032/PTL0GC1 provides better results than PTL060 for reasons which include that the active part of the molecule in PTL032/PTL0GC1 is a PAR-1 antagonist and also a PAR-2 agonist. These improved results show that PTL032/PTL0GC1 is inventive. Moreover, the inventor asserts that this PAR-1/PAR-2 property has not been recognised or exploited previously.

That some compounds can have a dual effect of PAR-1 antagonism and PAR-2 agonism is arguably disclosed in Kawabata et al 1999 (J Pharm and Exp Ther volume 288 pages 358-370). However, this paper does not disclose the compounds of the invention. Moreover this paper does not disclose anything about the medical applications and medical uses taught herein as part of the invention. In addition, the present application demonstrates improved results compared to unclaimed compounds. These results would not be expected. These results are surprising. The inventor asserts that these data are not only surprising but in fact are ‘astonishing’. The inventor asserts that these data change the complexion of knowledge how to address or treat the conditions disclosed herein. These data and assertions clearly evidence the inventive step (non-obviousness) of the appended claims.

In addition, although transgenic expression of anticoagulant activity at cell surfaces shows certain effects in the art, the therapeutic utility of intravenous administration of the compounds according to the present invention could not have been predicted. Among the reasons for this are included that because, following administration of the thromalexins intravenously, it could not be predicted that they would be transported to the vascular endothelium where the anti-atherosclerosis effect is needed. Although data showing that known compounds such as PTL004 can bind to red blood cells is disclosed, the compounds of the present invention are themselves completely novel compared to PTL004. It was not expected before the present invention that thrombalexins might work following intravenous administration. Moreover, the serious medical complications and drawbacks of the anticoagulant effect were revealed to be a significant challenge in this area of medicine and therefore would dissuade a skilled person from trying to follow this approach without knowledge of the invention.

Suitably the compound of the invention is for administration intravenously. Suitably the compound of the invention is administered intravenously. Suitably the method(s) of treatment of the invention comprise the step of administering a compound as described above intravenously. This further supports inventive step since intravenous administration of thrombalexins was not expected/predicted to work from a knowledge of the art.

In published documents where 3-mercaptopropionyl-Phe-Cha-Cha-Arg-Lys-Pro-Asn-Asp-Lys-NH₂ (i.e. the ‘active’ part of PTL032 and PTL0GC-1) has been referenced or used experimentally, the documents typically refer to this agent only as a PAR-1 antagonist. However, this compound actually has a dual action, acting as an agonist at PAR-2, whilst inhibiting PAR-1. This compound is one of many compounds mentioned in isolation in Kawabata et al 1999 (J Pharm and Exp Ther volume 288 pages 358-370), in particular at page 365 Right column:
“These data indicated that Mpr-NH2 was an agonist only for PAR2 and not for PAR1; and that Mpr-NH2 was a PAR1 antagonist.”

It should be noted that this disclosure differs from the present invention in many important respects, including for example that this compound mentioned in Kawabata et al 1999 is a free mercaptoproprionyl-peptide molecule and it has no linker (as required in the invention) and it has no cytotopic tail (as required in the invention) and there is no suggestion in Kawabata et al to attempt any modifications to this mercaptoproprionyl-peptide molecule which is indeed only used in Kawabata et al 1999 as a tool to exemplify their main idea which is the assay system for evaluating the biochemical activity of certain PAR agonists/antagonists disclosed by Kawabata et al. As well as these structural and intellectual differences between Kawabata et al and the present invention it is also important to note that nowhere in Kawabata et al is there any indication towards the medical applications which are a key part of the advances described herein.

In addition the inventors clearly assert that despite Kawabata et al 1999 mentioning the dual action (see above), (i.e. despite this fragment of the compound of the invention being mentioned as having dual action in isolation), all the reports in the art since have either ignored its dual action and/or not been aware of it. This is explained in more detail below.

Thus an important indication of the inventive step/non-obviousness of the present invention is not only about describing the dual action of one segment of the larger molecules of the invention, but more importantly the intellectual insights i.e. the idea/inspiration that this activity could be introduced into a very different molecular structure and the further insights that this could bring something unexpected and beneficial in terms of impact on disease.

The inventors assert that investigators in the field of the invention are unaware of the PAR-2 signalling capacity of this compound (as the inventors themselves were) compared to other PAR-1 antagonists. Certainly the dual action does not feature in studies disclosed in this field. The inventors assert that lack of acknowledgement of the dual action (even if appreciated) reflects the widely held view that PAR-1 and PAR-2 have similar functions and that signalling through each induces similar effects, even though each is activated by different proteases; in particular, thrombin (Factor IIa) cleaves PAR-1 but not PAR-2. For instance, it has been shown in a model of tumour metastases, that signalling through PAR-2 enhanced the effect of thrombin through PAR-1 on tumour cell migration (Shi et al 2004 Molecular Cancer Research vol 2 pages 395-402). Similarly, in a model of murine lung fibrosis, the pro-fibrotic impact of signalling through PAR-1 was dependent on PAR-2 signalling, showing that inhibition of either receptor inhibited development of fibrosis (Lin et al 2015 J Call Mol Med vol 19 pages 1346-1356). Similarly, in a model of vascular remodelling, PAR-1 and PAR-2 played a critical role in activation of adventitial fibroblasts, contributing to pathological vascular remodelling (He et al 2016 BBRC vol 473 pages 517-523). Most importantly, recent work in atherosclerosis-prone ApoE−/− mice (the same model as used by the inventors) suggested that signalling through PAR-2 contributed to the development of atherosclerosis, such that the absence of the receptor prevented plaque development (Jones et al 2018 Arterioscler Thromb Vase Biol 2018; 38(6):1271-1282).

This is a selection of examples which illustrate the common view in the art that both PAR-1 and PAR-2 contribute similarly to a broad range of pathologies, including atherosclerosis, the implication being that inhibition of either, or both, has a similar impact in preventing disease.

Therefore, it is established that a key idea underpinning the invention that providing PAR-2 stimulation whilst inhibiting PAR-1 would have potent anti-inflammatory properties is not predictable from the literature. In fact this goes against the view in the art. The inventors have gone against established thinking in arriving at the invention.

One of the inventive concepts is that this (dual signalling/provision of ‘conflicting’ PAR1 & PAR2 signals) is the basis of why 3-mercaptopropionyl-Phe-Cha-Cha-Arg-Lys-Pro-Asn-Asp-Lys-NH₂ has such potent impact in atherosclerosis.
This concept and the experimental evidence provided in support of it each establish inventive step/non-obviousness.

The inventors' evidence in support of this inventive concept is detailed here. First, in early mouse heart-to-rat transplant experiments, the inventors compared donor hearts from two strains, the first expressing a tissue factor inhibitor on endothelium, the second expressing hirudin on endothelium. In the former strain, as well as thrombin generation being prevented, both tissue factor and the generation of FXa were also inhibited (both can signal through PAR-2), so both PAR-2 and PAR-1 signalling was prevented. However, in the latter strain, only PAR-1 signalling by thrombin was inhibited as FXa was generated during rejection and able to signal through PAR-2. Allowing the provision of a PAR-2 signal whilst inhibiting PAR-1 was associated with a 50% reduction in monocyte infiltration into the transplanted hearts from the strain expressing hirudin, compared to the hearts expressing the tissue factor inhibitor. We refer to the examples section below for further evidence in support of this important new conceptual approach and inventive step/non-obviousness.

To date, there are few therapies that have been shown to cause such efficient regression of existing atheromatous lesions in mouse models of atheroma formation as the compounds as described above. Those known therapies that have caused regression (for example LXR agonists) have proven to be toxic in humans. Therefore the strengths of exemplary compounds of the invention such as PTL032/PTL0GC-1 include:
Magnitude of plaque regression comparable or higher than seen by any other intervention.
PTL032/PTL0GC-1 prevents AND treats atheromatous disease.
Cytotopic properties of PTL032/PTL0GC-1 endow them with potent anti-inflammatory but reduced anticoagulant/antiplatelet activity compared to parental peptides. This makes the new drugs safer for use in humans.
PTL032/PTL0GC-1's mechanism of action complements those of existing therapies.
PTL032/PTL0GC-1 are effective with intermittent dosing.
Cost of PTL032/PTL0GC-1 to be similar to small molecule drugs i.e. advantageously low cost compared to many biologic therapies.

### BRIEF DESCRIPTION OF THE DRAWINGS

Embodiments of the present invention will now be described further, with reference to the accompanying drawings, in which:

FIG. 1 shows photographs and bar charts: Inhibition of TF or thrombin on EC abolishes MIF expression in vascular wall and prevents formation of atheroma.

A-E. Three colour immunofluorescence images of sections through donor aortas, 6-12 weeks post-transplantation. Recipients were ApoE−/− mice, fed a high fat diet (HFD) for two weeks from age 6 weeks, prior to transplantation of aorta from CD31-TFPI-Tg (A,B), CD31-Hir-Tg (C-D) or C57BL/6 mice (E). Blue—nuclear stain 4′,6-diamidino-2-phenylindole (DAPI). Red—anti-hTFPI (A,B), anti-hirudin (Hir-C,D) or anti-CD31 (E). Green—MIF (A,C,E) or CD31 (B,D). Each panel of three images shows consecutive sections.
F-J—Analysis of atheroma development in whole aorta (F,G,H) and aortic root (H,I,J) after a HFD for 6 weeks post-transplantation. F&G: representative Oil Red O-stained en face preparations of aorta from ApoE−/− mice transplanted with aorta from CD31-TFPI-Tg (F) or BL/6 (G) mice. The transplanted section is highlighted by arrows. H: Quantitative assessments show the area occupied by atheroma, assessed at three different sites (as indicated) as a proportion of the total area (n=6 males each group) in ApoE−/− mice transplanted with aortas from CD31-TFPI-Tg (white bars) or BL/6 (grey bars) donors. Graphs show box plots with median with interquartile range (IQR) with whiskers showing upper and lower limits and outliers indicated as single data points. Means are represented with ‘x’. I&J Representative light photomicrographs of elastic/van Gieson stained sections from aortic root of mice transplanted with aortas from CD31-TFPI-Tg (I) or BL/6 (J) mice.
K-O—Analysis of atheroma development in the whole aorta (K, L,M) and aortic root (M, N, O) after a HFD for 12 weeks post-transplantation. K&L: representative Oil Red O-stained en face preparations of aorta from ApoE−/− mice transplanted with aorta from CD31-Hir-Tg (K) or BL/6 (L) mice. The transplanted section is highlighted by arrows. M: Quantitative assessments show the area occupied by atheroma, assessed at three different sites (as indicated) as a proportion of the total area (n=6 males each group) in ApoE−/− mice transplanted with aortas from CD31-Hir-Tg (white bars) or BL/6 (grey bars) donors. Graphs show box plots with median with interquartile range (IQR) with whiskers showing upper and lower limits and outliers indicated as single data points. Means are represented with ‘x’. N&O representative light photomicrographs of elastic/van Gieson stained sections from aortic root of mice transplanted with aortas from CD31-Hir-Tg (N) or BL/6 (O) mice. Quantitative analyses were performed by a member of the team blinded to the mouse strain.

FIG. 2 shows photographs, graphs and charts: Impact of IV PTL060.

A&B: Two colour IF images of cross sections through aorta harvested at 6 hours post-IV injection of 10 mg/g PTL060 (A) or equimolar (5 mg/g) HLL (B) stained with isotype control or RICS2 antibody (which recognises HLL) as indicated. Blue-DAPI. (NB Sections examined at all other time points showed less evidence of binding by PTL060).
C-E: Flow cytometric assessment of binding to erythrocytes (C), CD11b+ leukocytes (D) and platelets (gated on CD41+) (E) obtained from mice given either saline control, HLL (2.5 mg/g), or PTL060 (5 mg/g). Graphs show percentage of population binding RICS antibody (left column) and the geometric mean of the fluorescence intensity of binding (right column). Samples were taken from mice at the time points post-injection as indicated. n=3 per group.
F&G: Thrombin clotting times (seconds f SEM) in plasma. Blood was collected into citrated tubes at the times specified under terminal anaesthesia before spinning at 15000 g for 10 minutes to separate out cellular components and plasma. Thrombin times performed by adding 25 U (F) or 50 U (G) thrombin to 100 ml of plasma and recording time for a fibrin clot to form. Mice (n=3 per group) injected with PTL060 (5 mg/g—filled squares) or equimolar dose of HLL (2.5 mg/g—circles). Plasma from mice treated with PTL060 was centrifuged for a further 20 minutes at 10000 g, to remove any membrane bound PTL060, before repeating assessments (open squares).
H: Graph depicting tail bleeding times in minutes f SEM at various times after IV injection of control phosphate buffered saline (open circles), PTL060 10 mg/g (squares) or equimolar (5 mg/g) HLL (closed circles). N=6 per group. Mouse euthanised at 20 minutes if tail still bleeding.

FIG. 3 shows photographs and bar charts: IV PTL060 inhibits MIF and prevents atherosclerosis

A: Quantitative impact of PTL060 on MIF expression by endothelium (left axis), represented as the proportion of CD31+ cells staining for MIF, plotted against time or development of atheroma (right axis) 4 weeks post injection. Mice (n=6) given either PBS control (white) or PTL060 10 mg/g (grey) by IV injection, 2 weeks after starting a HFD and analysed at the time points indicated. Graphs show box plots with median with interquartile range (IQR) with whiskers showing upper and lower limits and outliers indicated as single data points. Means are represented with ‘x’.
B&C: Representative light photomicrographs of elastic/van Gieson stained sections from aortic root of ApoE−/− mice treated with PBS (B) or 10 mg/g PTL060 (C).
D: Quantitative impact of PTL060 on MIF expression 1-week post injection (left axis), represented as the proportion of CD31+ cells staining for MIF, or development of atheroma (right axis) 4 weeks post injection. Mice (n=6) given either PTL060 2.5 mg/g (white bars), PTL060 5 mg/g (grey bars), PTL060 10 mg/g (striped bars) or HLL 5 mg/g (diamond bars) by IV injection, 2 weeks after starting a HFD and analysed at the time points indicated. HLL 5 mg/g is equimolar to PTL060 10 mg/g. Graphs show box plots with median with interquartile range (IQR) with whiskers showing upper and lower limits and outliers indicated as single data points. Means are represented with ‘x’.
E-H: Representative light photomicrographs of elastic/van Gieson stained sections from aortic root of ApoE−/− mice treated with PTL060 2.5 mg/g (E), 5 mg/g (F), 10 mg/g (G), or HLL 5 mg/g (H).

FIG. 4 shows photographs and bar charts: IV PTL060 causes regression of atherosclerosis

A-D: Representative Oil Red O-stained en face preparations of aorta from ApoE−/− mice fed a HFD from age of 6-22 weeks (baseline: A), or 6-28 weeks with weekly injections (weeks 23-28) of saline (B), control cytotopic ‘tail’ compound (C) or PTL060 10 mg/g (D).
E-H: Representative light photomicrographs of elastic/van Gieson stained sections from aortic root of ApoE−/− mice fed a HFD from age of 6-22 weeks (baseline: E), or 6-28 weeks with weekly injections (weeks 23-28) of saline (F), control cytotopic ‘tail’ compound (G) or PTL060 10 mg/g (E).
I: Qualitative comparison of impact of PTL060 on atheroma formation in mice on HFD aged 6-22 weeks (white bars) or 6-28 weeks with weekly injections (weeks 23-28) of saline (grey bars), control ‘tail’ compound (striped bars) or PTL060 10 mg/g (diamond bars).
J-M: Representative Oil Red O-stained en face preparations of aorta from ApoE−/− mice fed a HFD from age of 6-22 weeks (Baseline: J), or 6-28 weeks with weekly injections (weeks 23-28) of saline (K), control ‘untailed’ HLL (L) or PTL060 10 mg/g (M).
N-Q: Representative light photomicrographs of elastic/van Gieson stained sections from aortic root of ApoE−/− mice fed a HFD from age of 6-22 weeks (N), or 6-28 weeks with weekly injections (weeks 23-28) of saline (O), control untailed HLL (P) or PTL060 10 mg/g (Q).
R: Qualitative comparison of impact of PTL060 on atheroma formation in mice on HFD aged 6-22 weeks (white bars) followed by weekly injections, for 6 weeks of saline (grey bars), control untailed HLL (striped bars) or PTL060 10 mg/g (diamond bars).
S-T: Impact of PTL060 on foam cells in atherosclerosis. Representative light photomicrographs of elastic/van Gieson stained sections from aortic root (S) with consecutive sections analysed by two-colour immunofluorescence (T) stained with DAPI (blue) or anti-CD68 (green). ApoE−/− mice were fed a HFD from age of 6-22 weeks, followed by weekly injections, for 6 weeks of saline, control untailed HLL or PTL060 10 mg/g as indicated.
U: Graphical representations of the % of plaque area staining with Oil Red O (upper panel) and, in lower panel, the % of area occupied by CD68+ cells (white bars) with the proportion of those CD68+ cells co-localising with lipid (grey bars). Each graph is a box plot with median with interquartile range (IQR) with whiskers showing upper and lower limits and outliers indicated as single data points. Means are represented with ‘x’. Data is derived from an assessment of each of the three aortic root plaques from 6 individual mice, from consecutive sections as illustrated in S&T.
V-W: Representative Oil Red O-stained en face preparations of aorta from ApoE−/− mice fed a normal chow diet to the age of 28 weeks, followed by weekly injections, for 6 weeks of saline (V) or PTL060 10 mg/g (W).
X-Y: Representative light photomicrographs of elastic/van Gieson stained sections from aortic root of ApoE−/− mice fed a chow diet age to the age of 28 weeks, followed by weekly injections, for 6 weeks of saline (X) or PTL060 10 mg/g (Y).
Z: Qualitative comparison of impact of PTL060 on atheroma formation in mice on chow diet to for 28 weeks followed by weekly injections, for 6 weeks of saline (white bars) or PTL060 10 mg/g (grey bars).

FIG. 5 shows photographs and bar charts: Phenotype of plaque cells after PTL060 Three colour immunofluorescence images show confocal microscopic analysis of consecutive sections of aortic roots of ApoE−/− mice, fed a high fat diet from 6 to 22 weeks (‘Baseline’ A, K,O) or 6-28 weeks, with mice administered weekly injections of saline (B,L,P), HLL (C,M,Q), or PTL060 (D,N,R) as indicated between weeks 22-28. Panels show the plaque expression of CD68 (red) with (green) either MIF (A-D) CCR7 (K-N) or ABCA1 (O-R). Yellow in overlay image indicates co-localisation. The plaque area is demarcated by the lumen (L) and the dotted white line. Le=aortic leaflet. Each panel of images is accompanied by graphical representations of the % of plaque area staining for the molecule of interest (E-MIF, H-CCR7, S-ABCA1) and the % of plaque area occupied by CD68+ (F, I, T) and the proportion of CD68+ cells (white bars) and CD68-negative cells (grey bars) co-staining for MIF (G), CCR7 (J), or ABCA1 (U). Each graph is a box plot with median with interquartile range (IQR) with whiskers showing upper and lower limits and outliers indicated as single data points. Means are represented with ‘x’. Each is derived from an assessment of each of the three aortic root plaques from 6-24 individual mice.

FIG. 6 shows photographs and bar charts: Impact of adoptive transfer of CD11b+ cells expressing tethered thrombin inhibitor

All panels: CD11b cells, harvested from either BL/6 or CD31-Hir-Tg mice were labelled in vitro with PKH26 (red) and adoptively transferred into ApoE−/− mice fed a HFD between ages of 6-22 weeks. Aortic roots were collected 48 hours post-injection, for confocal IF analysis of the phenotype of adoptively transferred cells. Graphs are a box plot with median with interquartile range (IQR) with whiskers showing upper and lower limits and outliers indicated as single data points. Means are represented with ‘x’. Each is derived from an assessment of at least 3 aortic root plaques from 6-35 individual mice.
A: To illustrate the expression of MIF (green) at baseline age 22 weeks, throughout the plaque area in a mouse that received BL/6 CD11b+ cells.
B-D: Comparison of the recruitment of CD11b+ cells from BL/6 (B) and CD31-Hir-Tg (C) mice. Hirudin (green) only seen in cells from CD31-Hir-Tg mice. D illustrates quantitative assessment of the proportion of plaque area occupied by PKH26+ cells.
E-G: To illustrate expression of Ly6G (green) within the plaque after adoptive transfer of CD11b+ cells from BL/6 (E) or CD31-HIr-Tg (F) mice. G illustrates quantitative assessment of the proportion of PKH26+ cells co-expressing Ly6G.
I-J: To illustrate expression of CCR2 (green) within the plaque after adoptive transfer of CD11b+ cells from BL/6 (H) or CD31-HIr-Tg (I) mice. J illustrates quantitative assessment of the proportion of PKH26+ cells co-expressing CCR2.
K-M: To illustrate expression of ABCA1 (green) within the plaque after adoptive transfer of CD11b+ cells from BL/6 (K) or CD31-HIr-Tg (L) mice. M illustrates quantitative assessment of the proportion of PKH26+ cells co-expressing ABCA.
N-P: To illustrate expression of CCR7 (green) within the plaque after adoptive transfer of CD11b+ cells from BL/6 (N) or CD31-HIr-Tg (O) mice. P illustrates quantitative assessment of the proportion of PKH26+ cells co-expressing CCR7.

FIG. 7 shows photographs and bar charts: Monocyte recruitment and phenotype after systemic PTL060.

Confocal microscopic analysis of three colour immunofluorescence images through consecutive sections of aortic roots of ApoE−/− mice, fed a high fat diet from 6 to 26 weeks, with mice administered weekly injections of saline or PTL060 as indicated below between weeks 22-25. 1 week after the last injection, mice were injected with PKH2-labelled CD11b cells (green) and aortic roots harvested 48 hours later. Graphs are a box plot with median with interquartile range (IQR) with whiskers showing upper and lower limits and outliers indicated as single data points. Means are represented with ‘x’. Each is derived from a double assessment of each of the three aortic root plaques from 3 individual mice.
A-C: To illustrate the expression of MIF (red) after adoptive transfer of BL/6 CD11b+ cells in mice treated with saline (A) or PTL060 (B). C illustrates quantitative assessment of the proportion of plaque area occupied by PKH2+ cells. D-F: To illustrate the expression of CCR2 (red) after adoptive transfer of BL/6 CD11b+ cells in mice treated with saline (D) or PTL060 (E). F illustrates quantitative assessment of the proportion of PKH2+ cells co-expressing CCR2. G-I: To illustrate the expression of CCR7 (red) after adoptive transfer of BL/6 CD11b+ cells in mice treated with saline (G) or PTL060 (H). I illustrates quantitative assessment of the proportion of PKH2+ cells co-expressing CCR7. J-K: To illustrate the expression of ABCA1 (red) after adoptive transfer of BL/6 CD11b+ cells in mice treated with saline (J) or PTL060 (K). L illustrates quantitative assessment of the proportion of PKH2+ cells co-expressing ABCA1.

FIG. 8 shows photographs and bar charts: Regression induced by thrombin inhibitor on isolated CD11b+ cells

Samples represented here are from ApoE−/− mice fed a HFD from age of 6-28 weeks with weekly (weeks 23-28) injections of CD11b+ cells from BL/6 mice pre-incubated with saline (A, E), control ‘tail’ molecule (B, F), PTL060 10 mg/g (C,G) or with CD11b cells from CD31-Hir-Tg mice (D, H)
A-D: Representative Oil Red O-stained en face preparations of aorta
E-H: Representative light photomicrographs of elastic/van Gieson stained sections from aortic root
I: Qualitative comparison of atheroma regression in the whole aorta (en face) or aortic root of mice fed a HFD from age of 6-28 weeks with weekly (weeks 23-28) injections of CD1b+ cells from BL/6 mice pre-incubated with saline (white bars), control ‘tail’ molecule (grey bars), PTL060 10 mg/g (striped bars) or with CD1b cells from CD31-Hir-Tg mice (diamond bars).
J: Graph is a box plot with median with interquartile range (IQR) with whiskers showing upper and lower limits and outliers indicated as single data points. Means are represented with ‘x’. Each is derived from an assessment of at least 3 aortic root plaques from 6-24 individual mice. It illustrates the proportion of plaque area occupied by cells expressing the various markers (as indicated on abscissa) from mice receiving CD11b+ cells from BL/6 mice pre-incubated with saline (white bars) or CD31-Hir-Tg mice (grey bars).

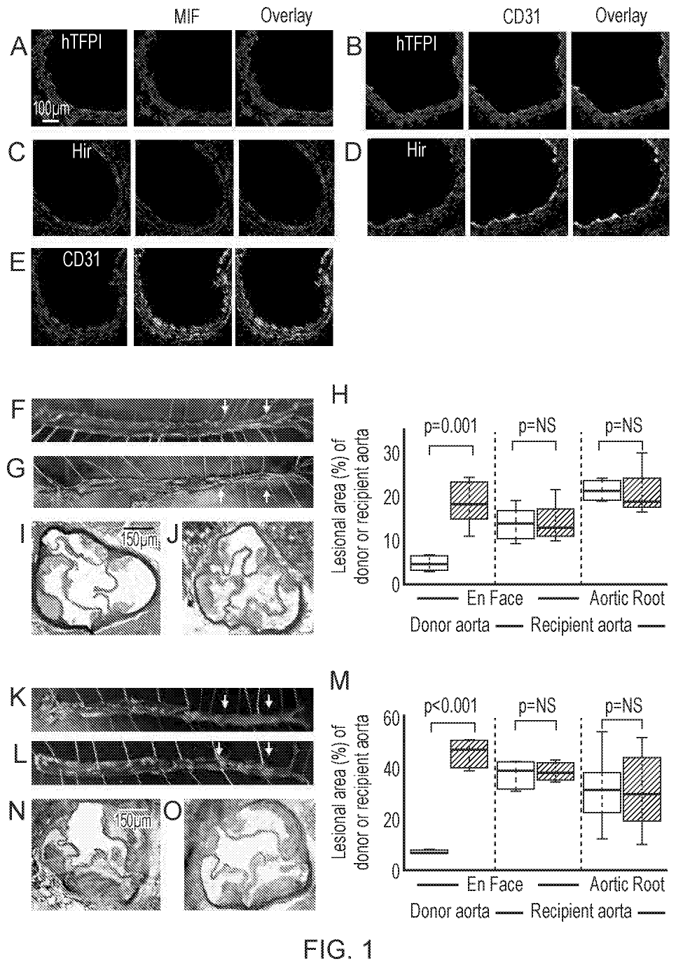

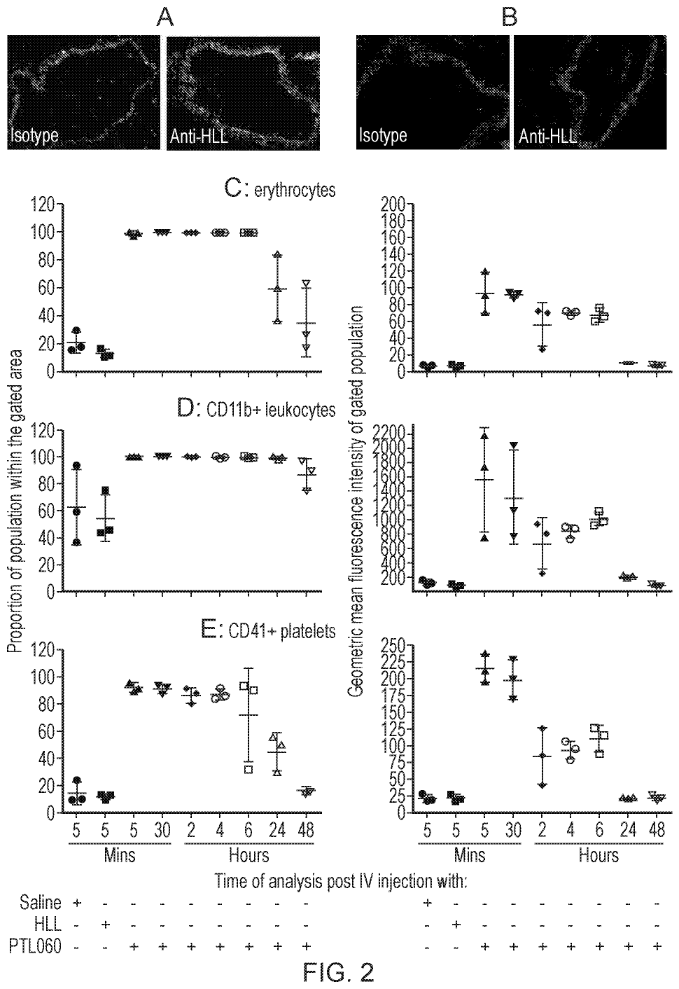

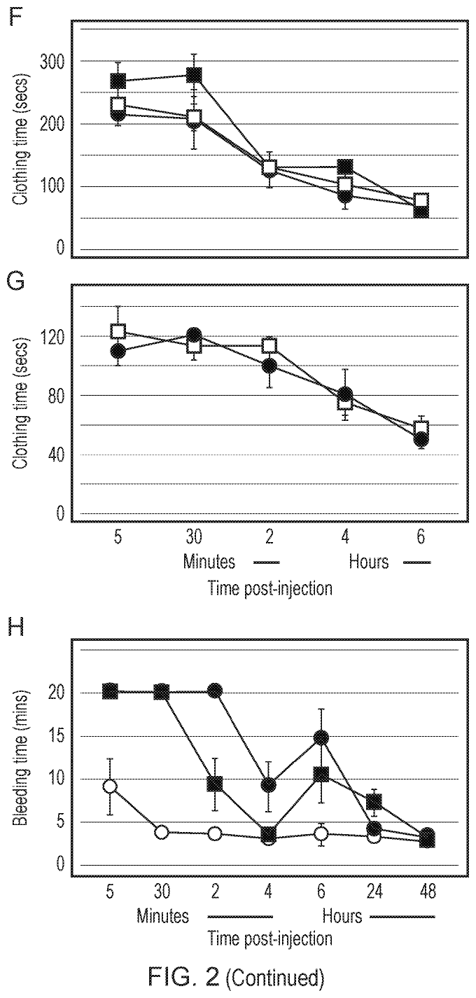

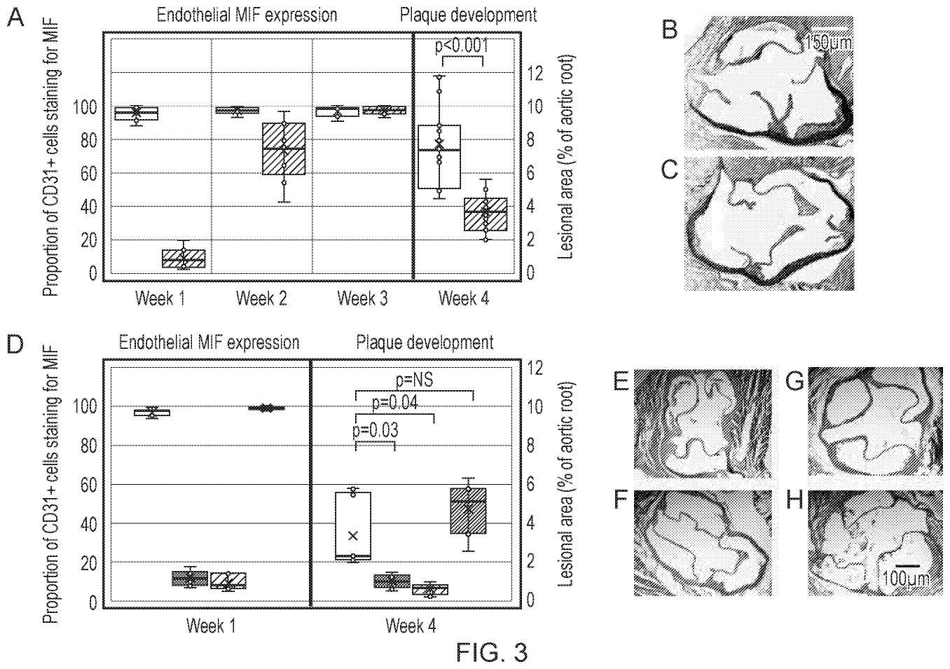

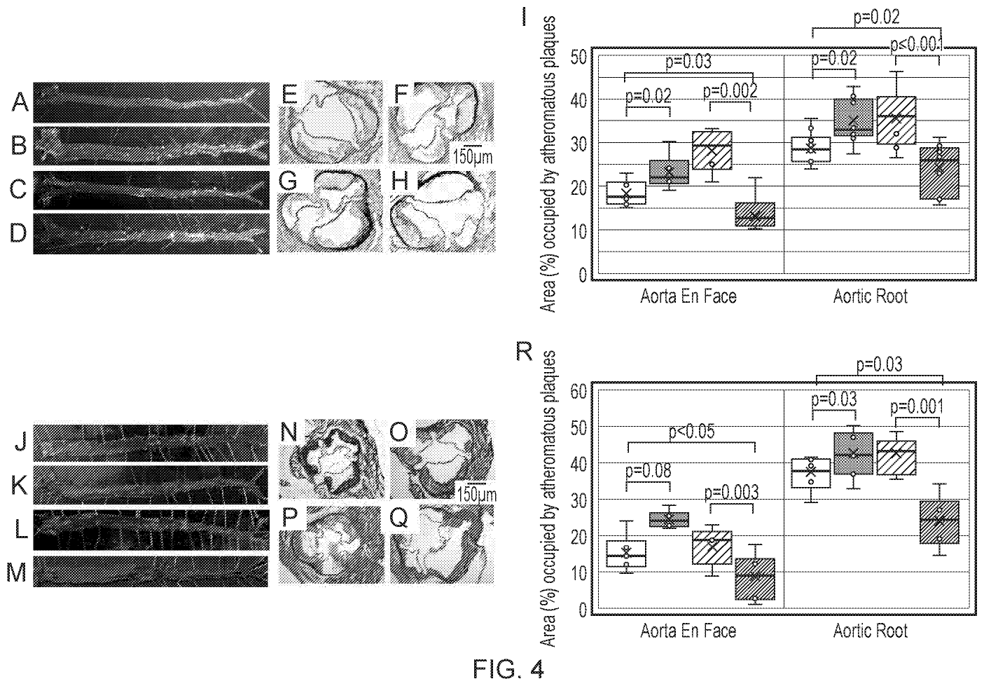

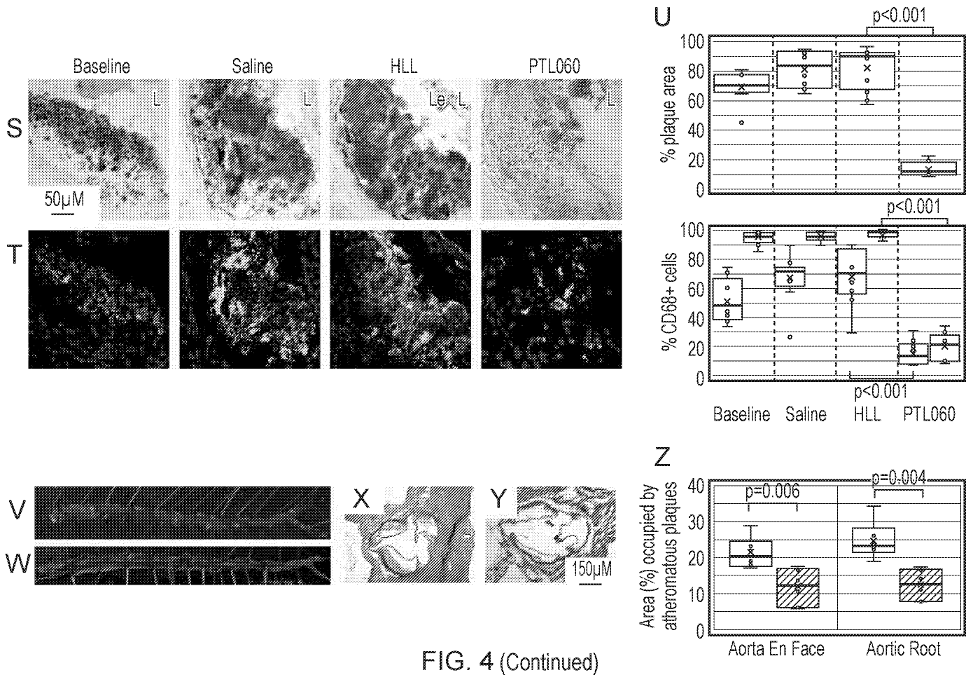

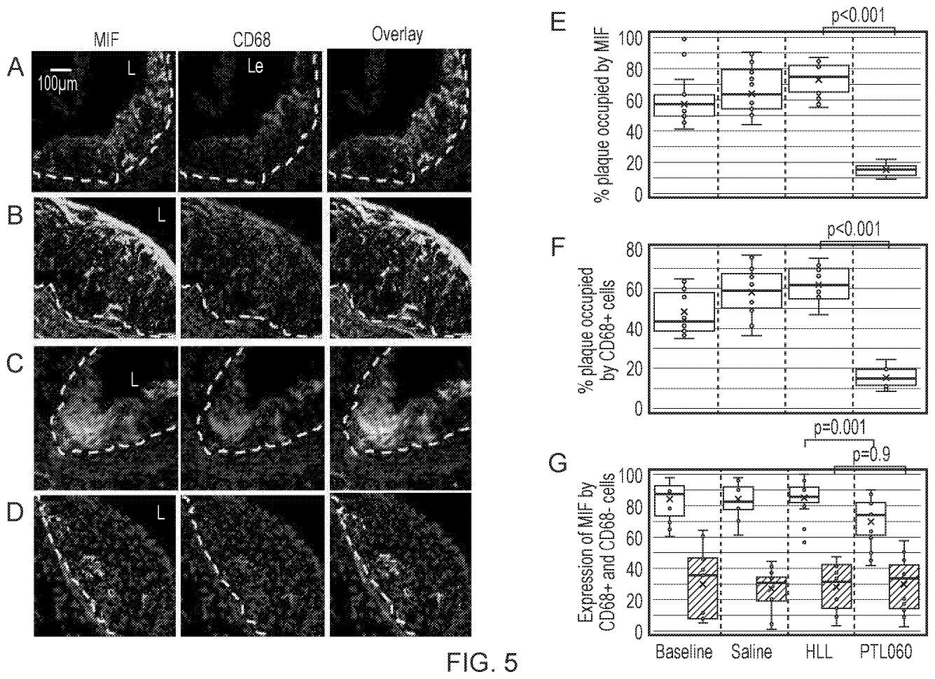

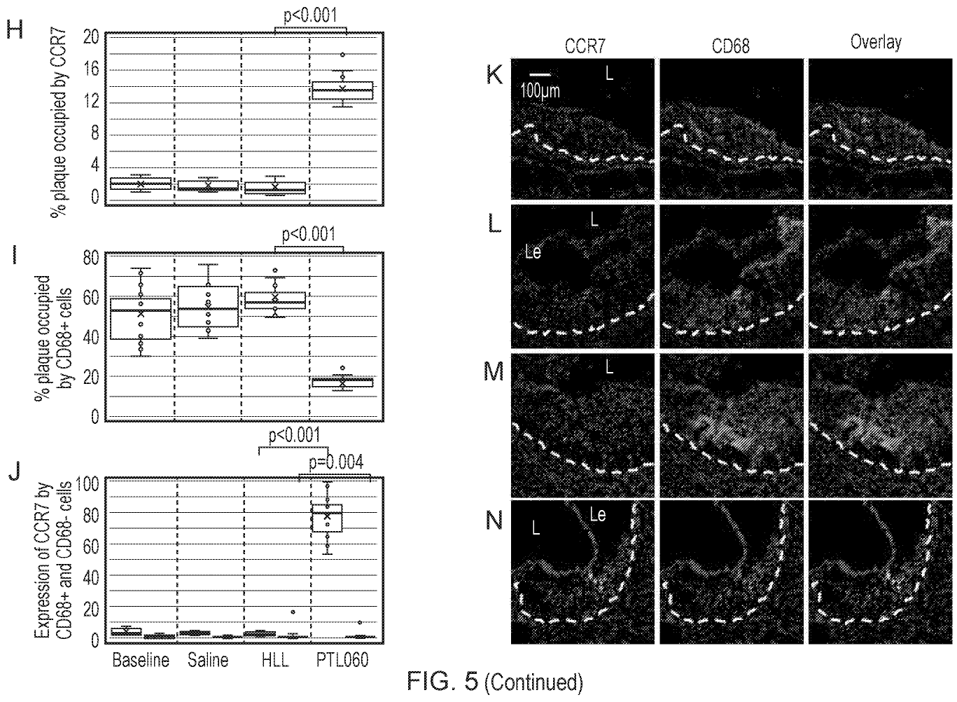

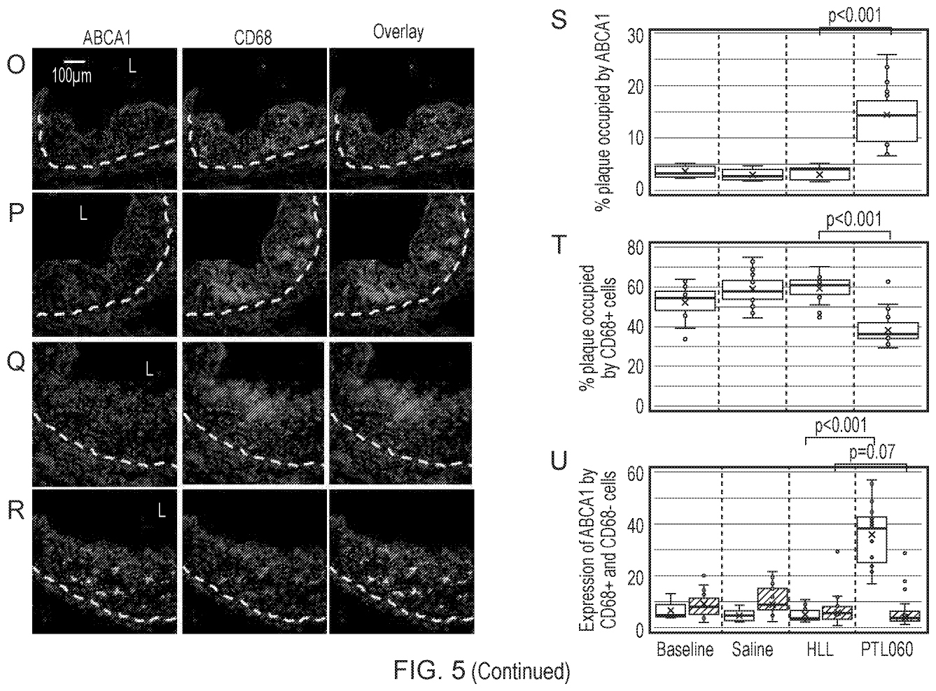

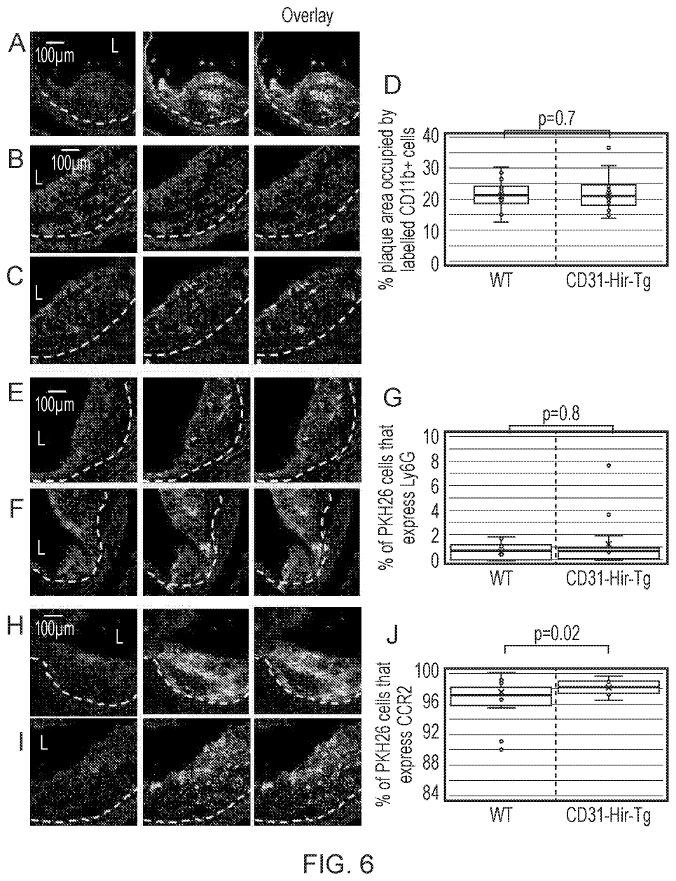

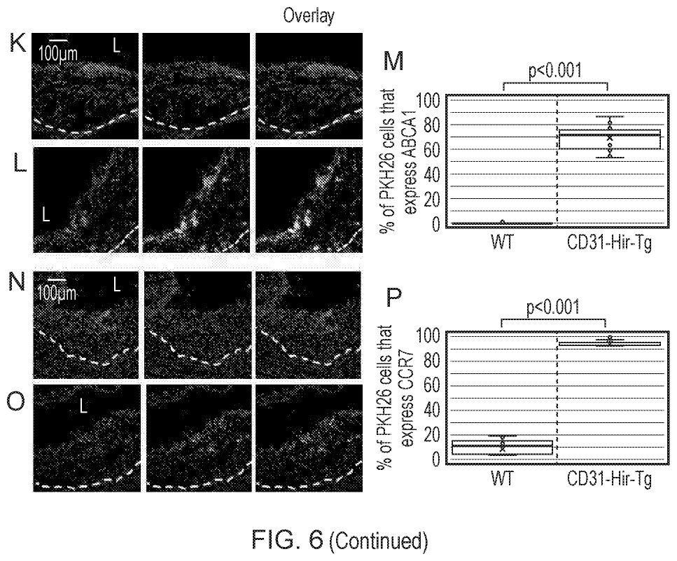

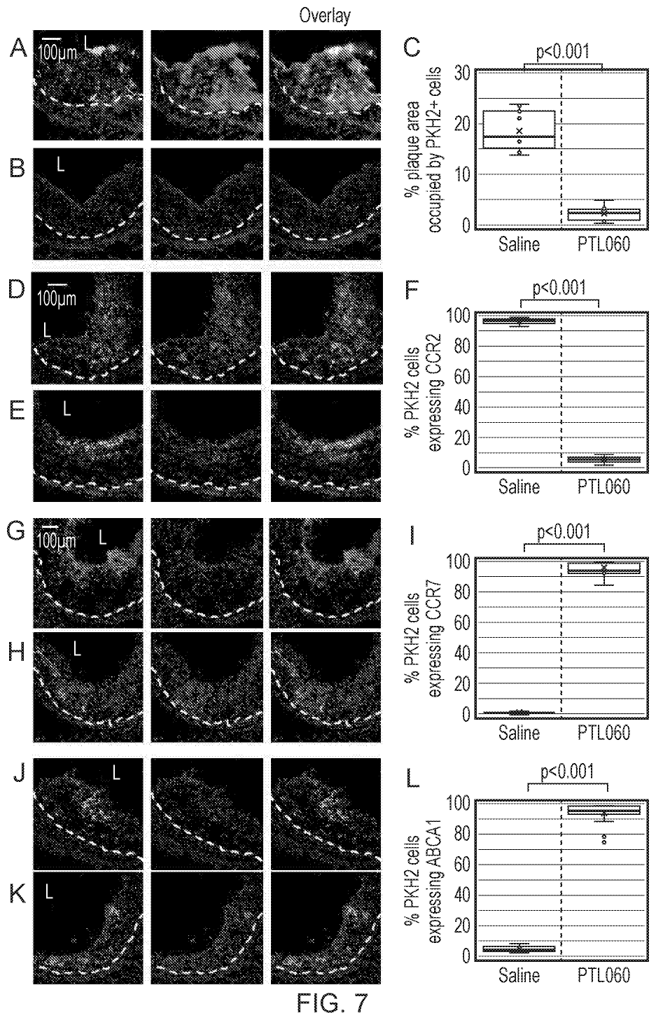

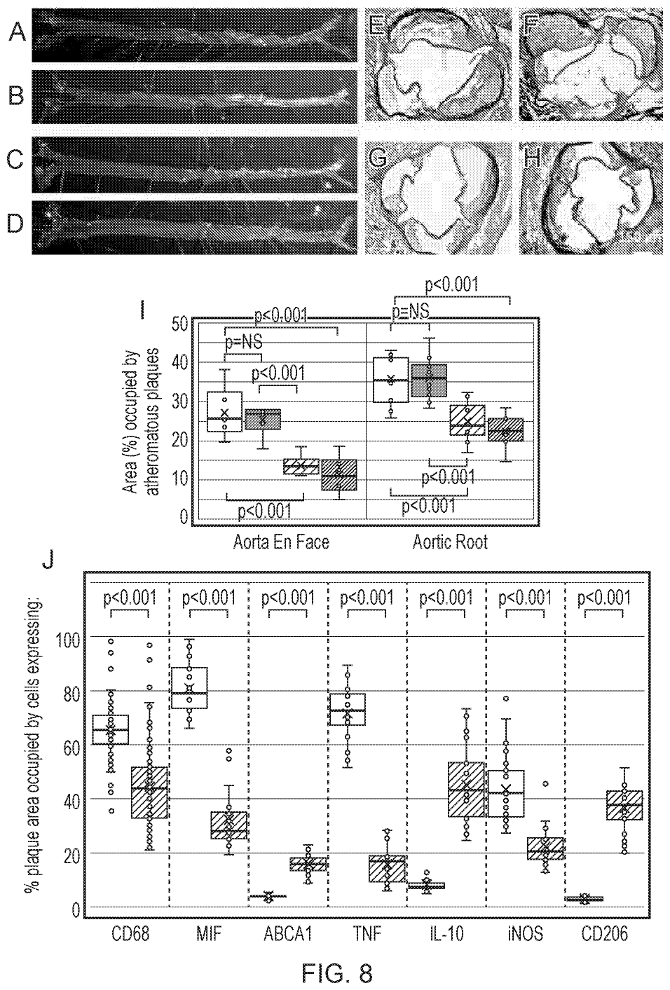

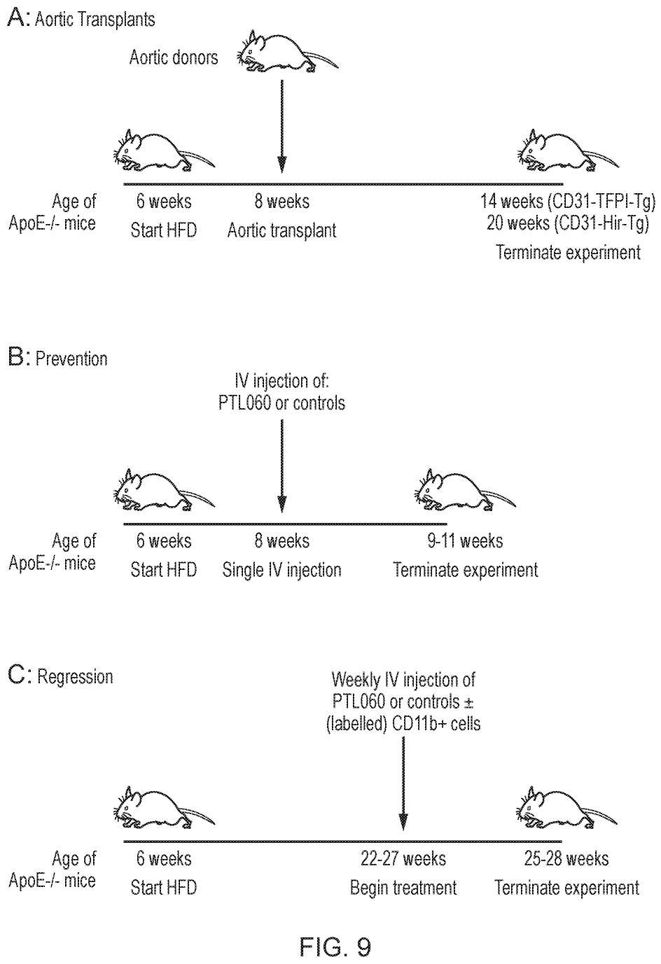

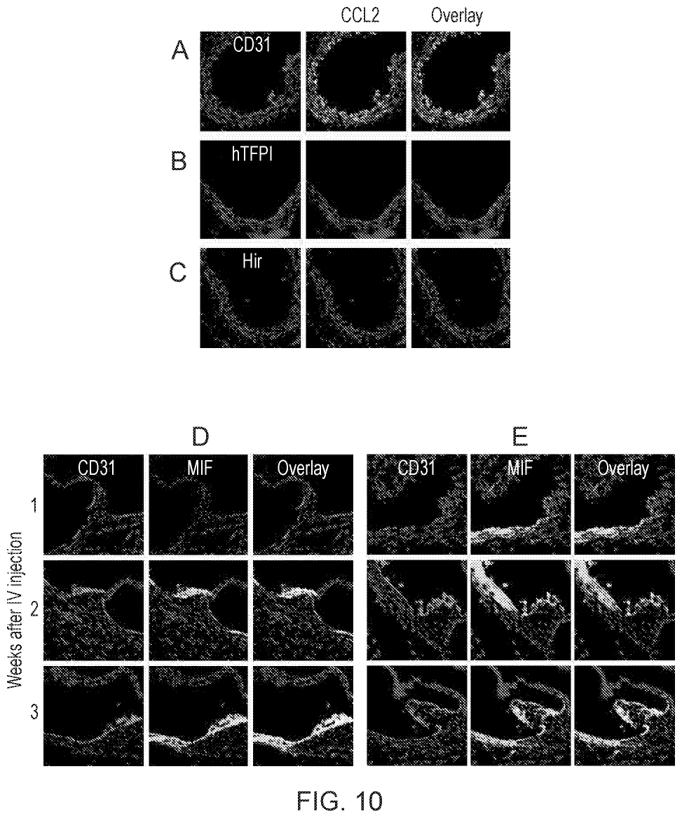

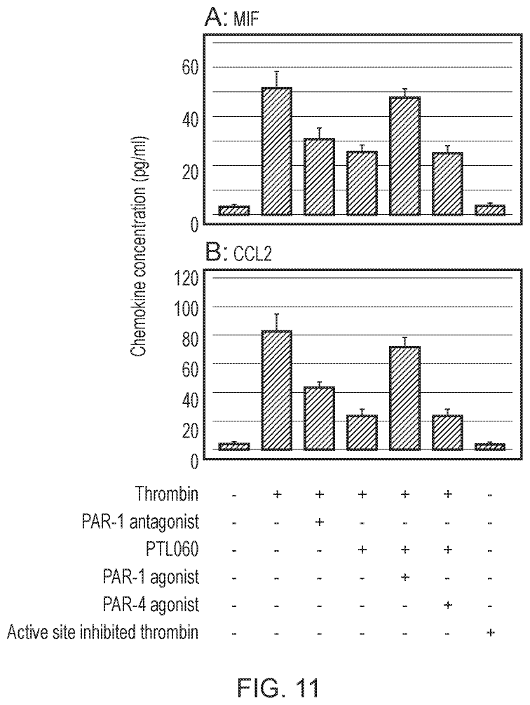

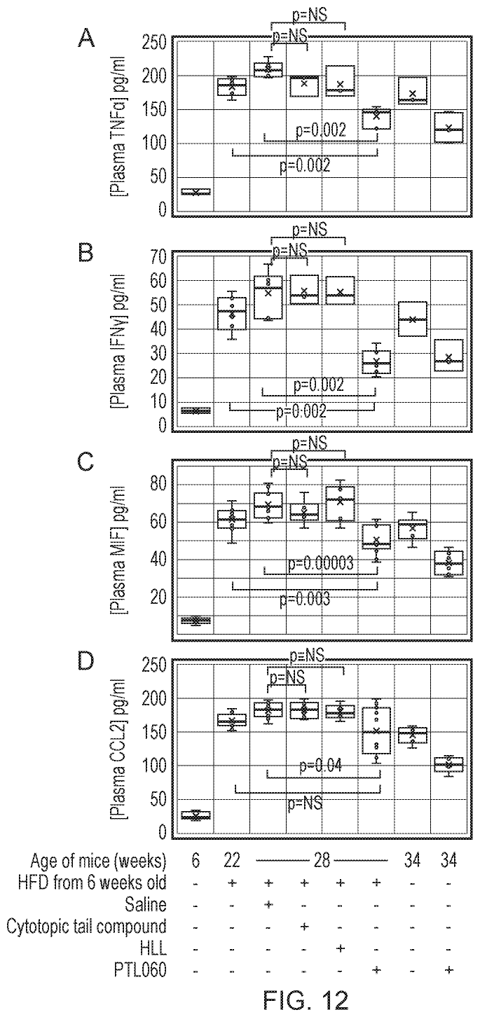

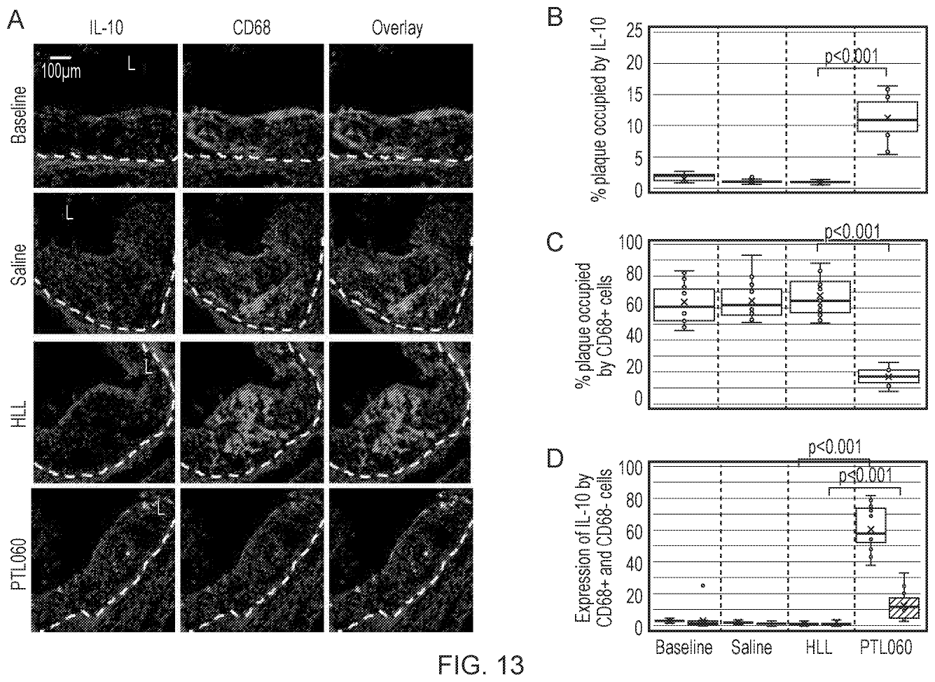

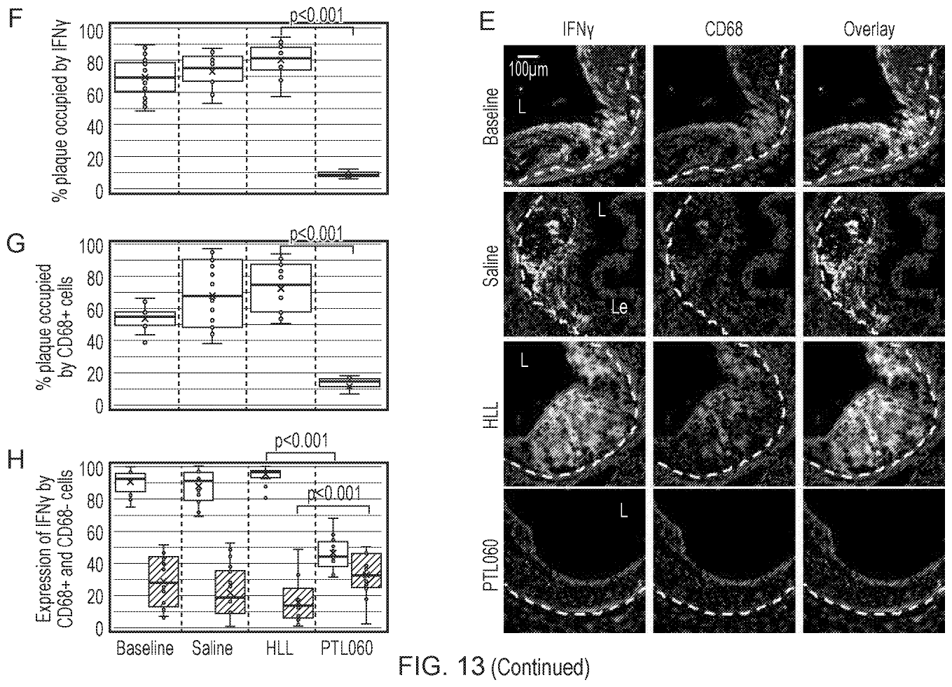

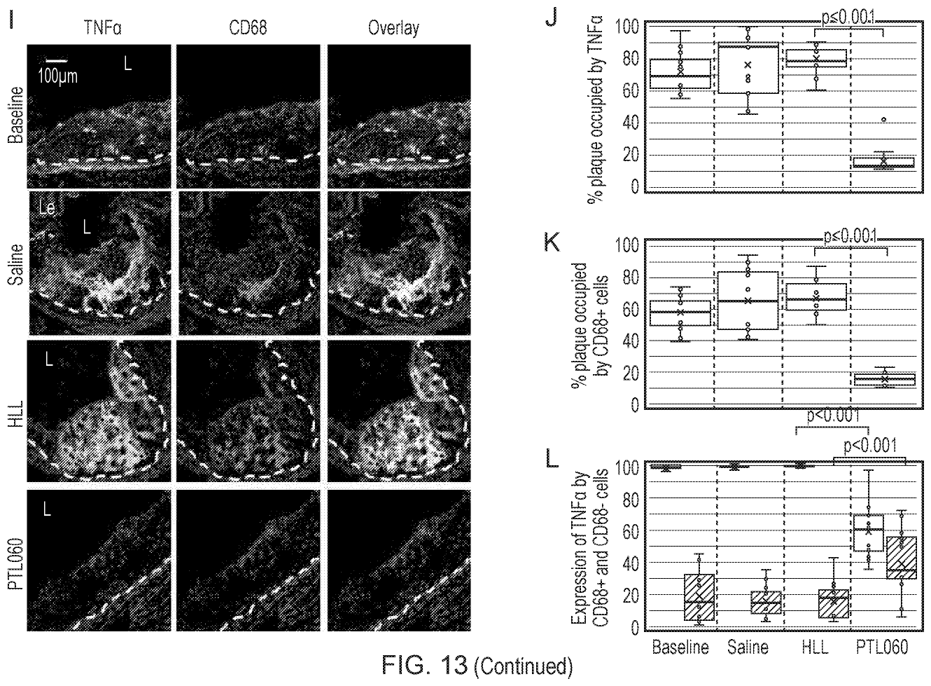

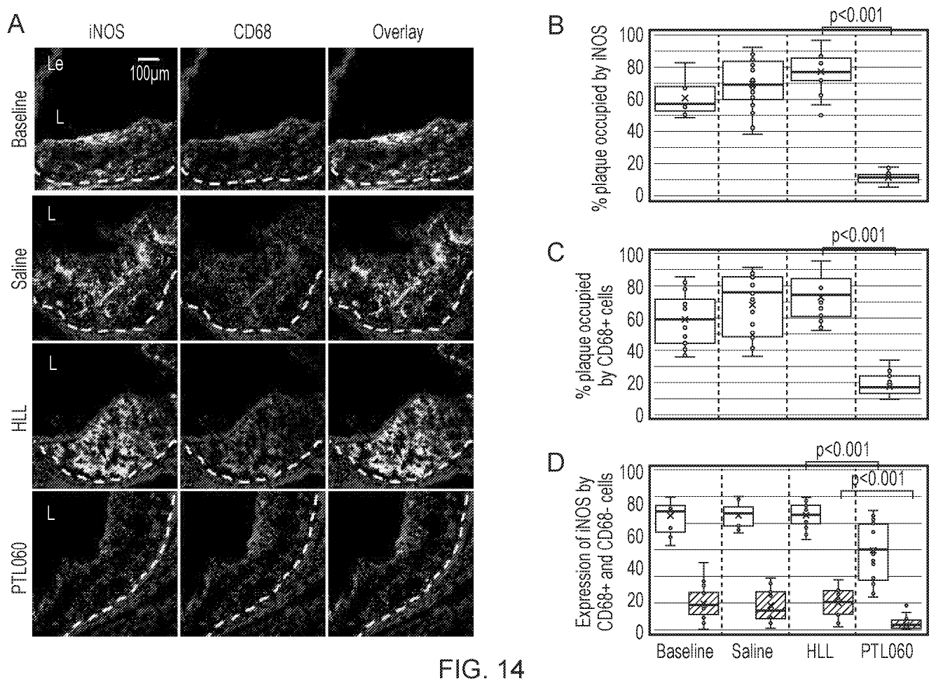

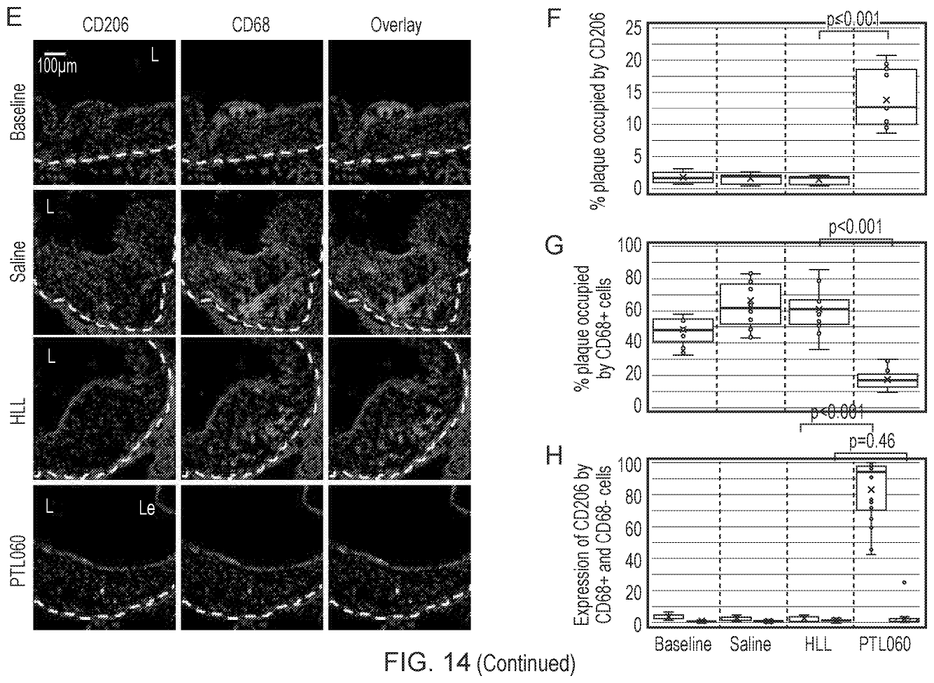

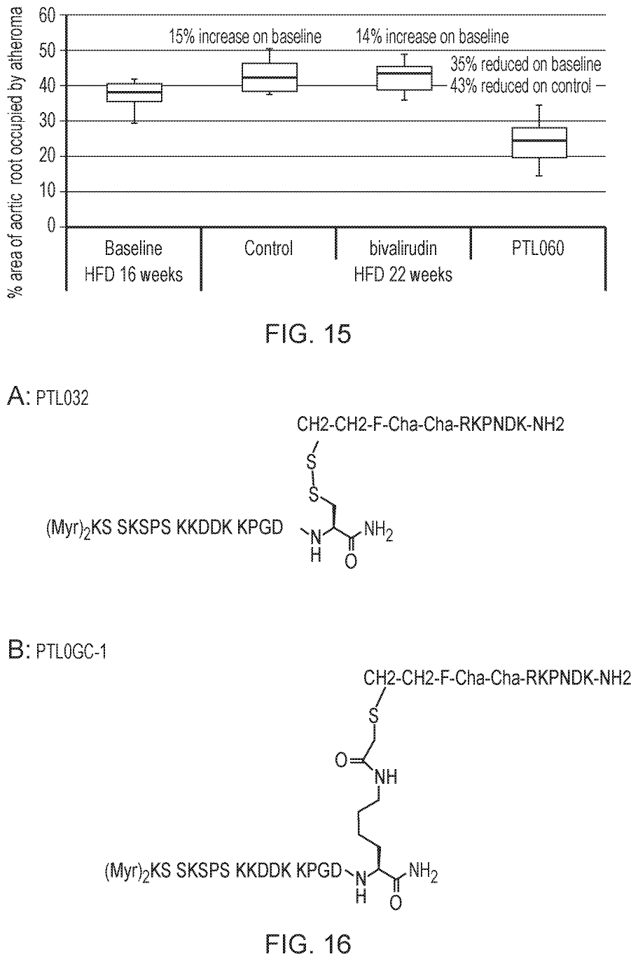

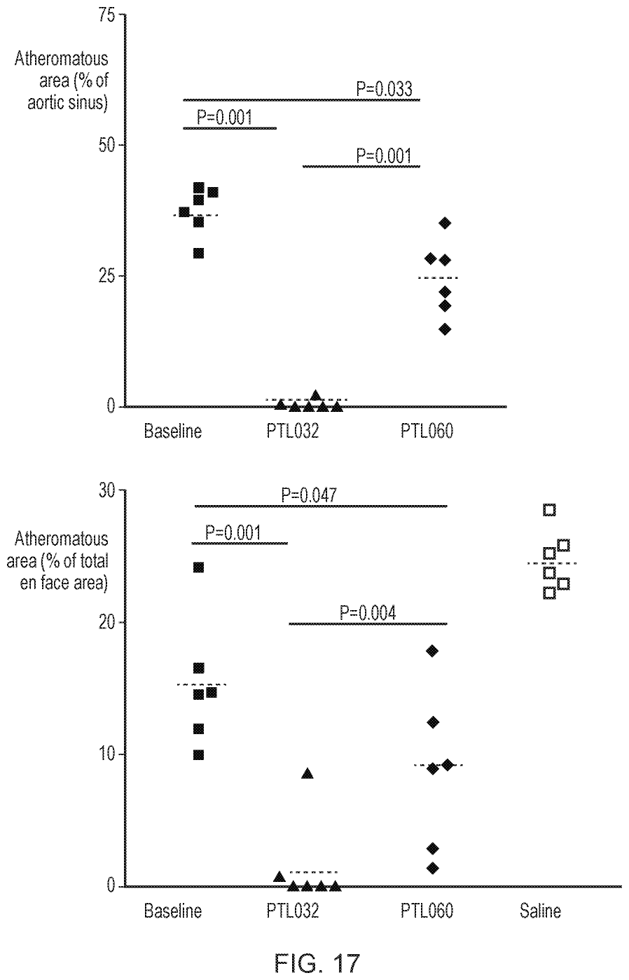

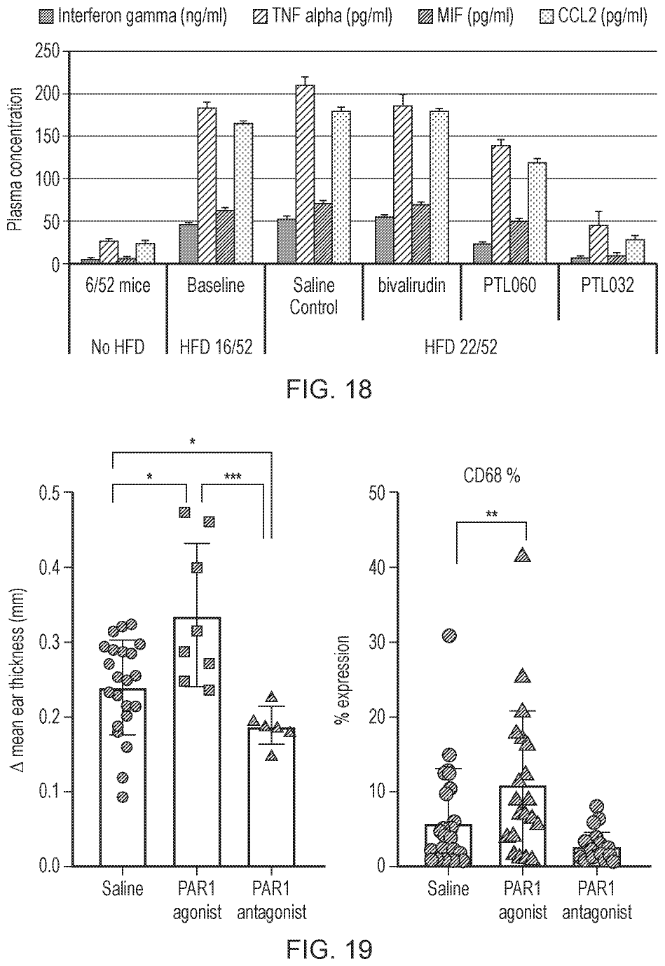

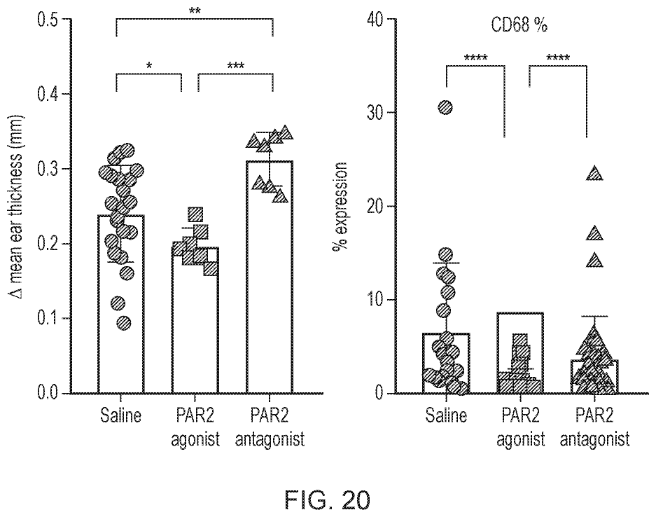

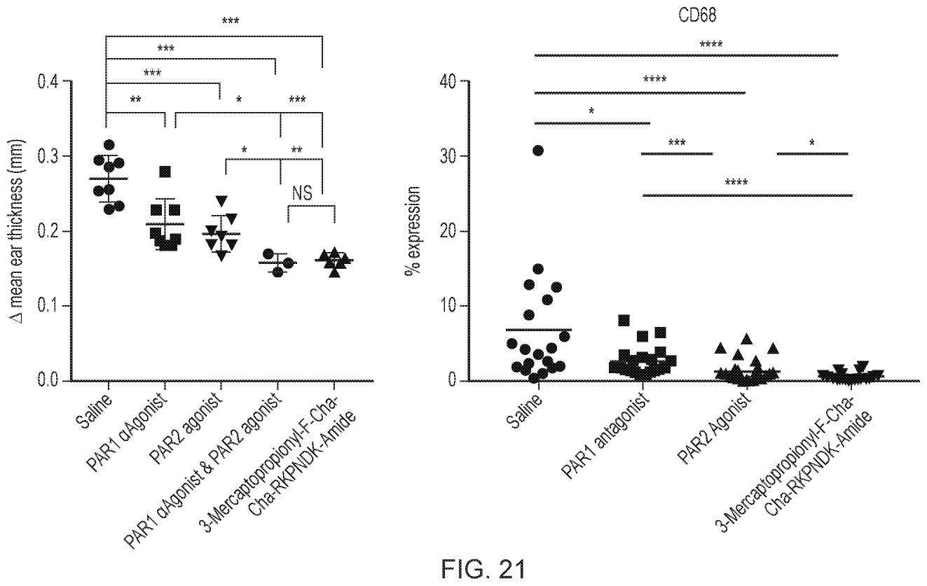

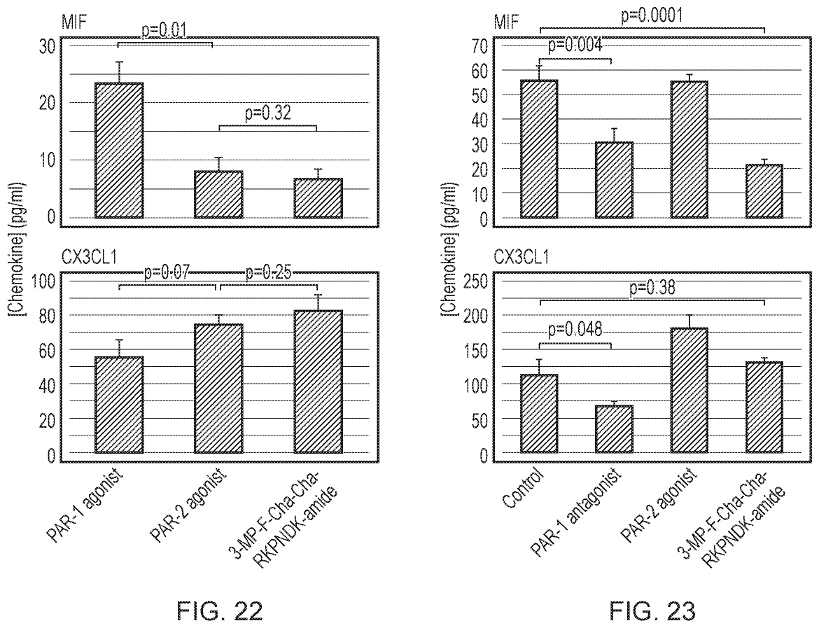

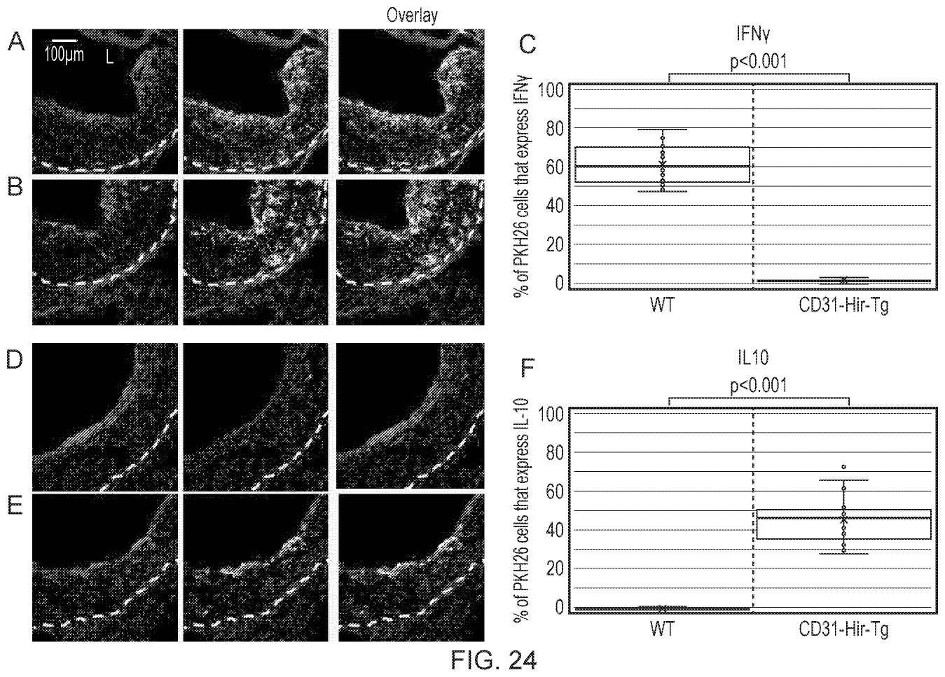

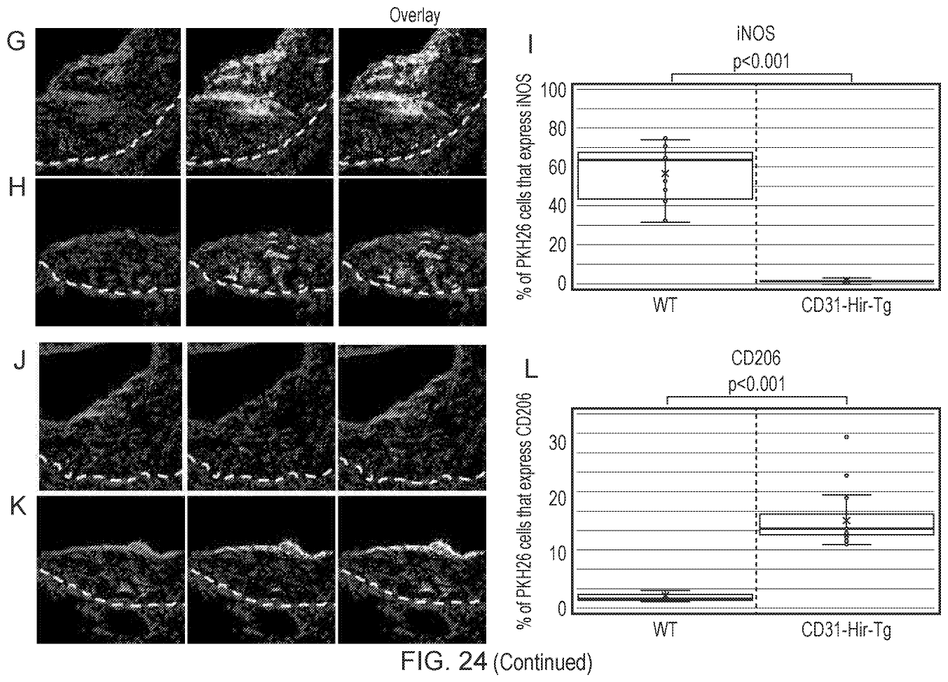

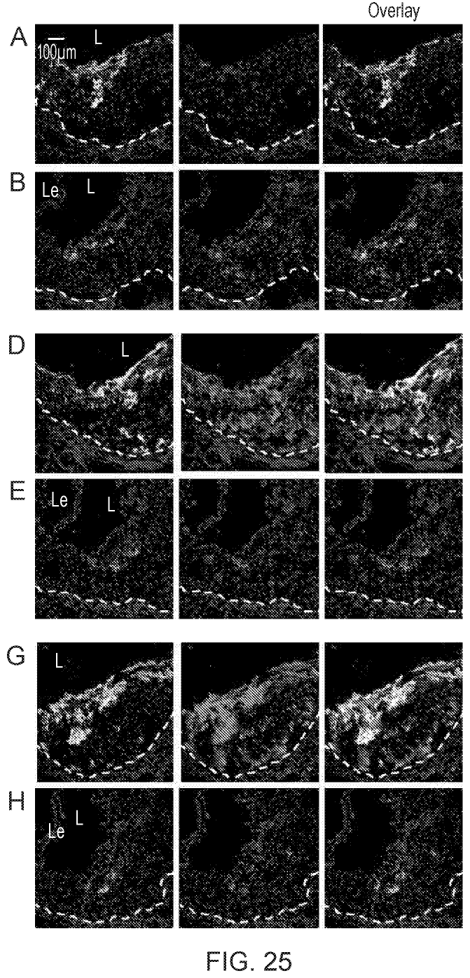

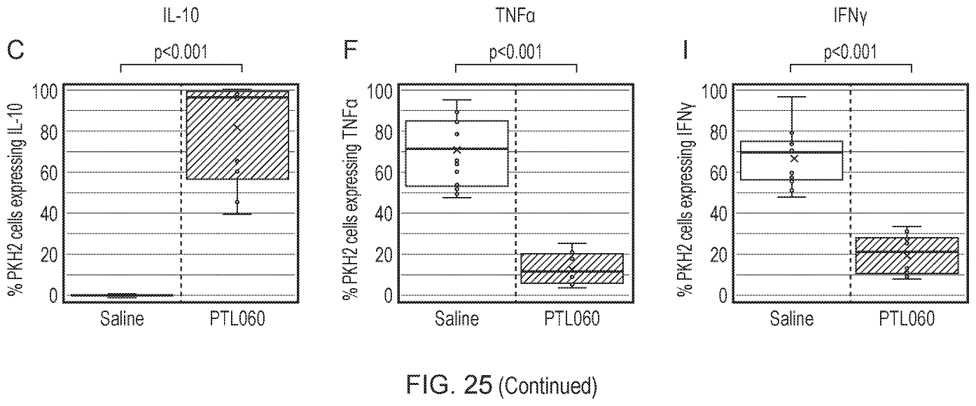

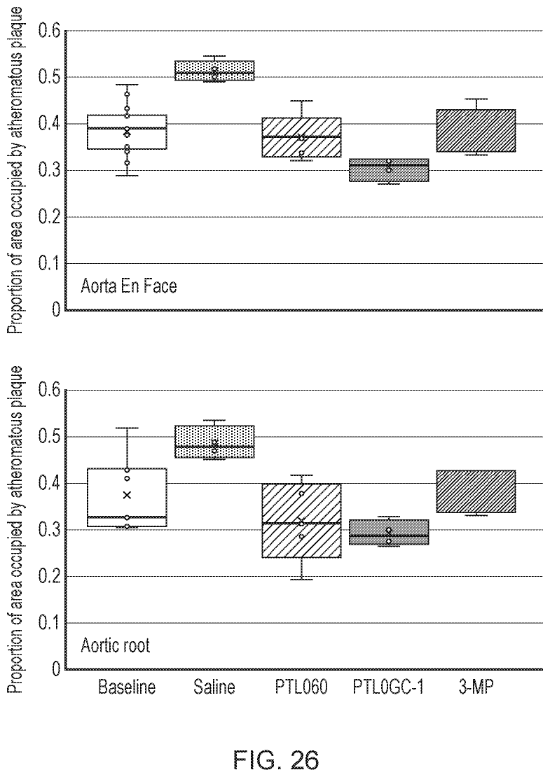

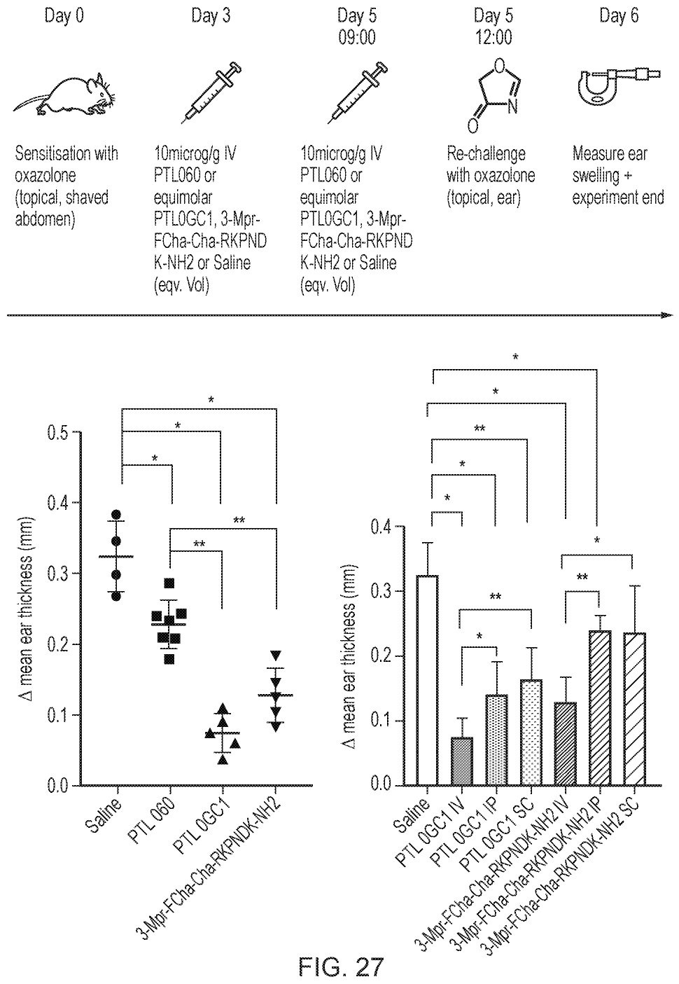

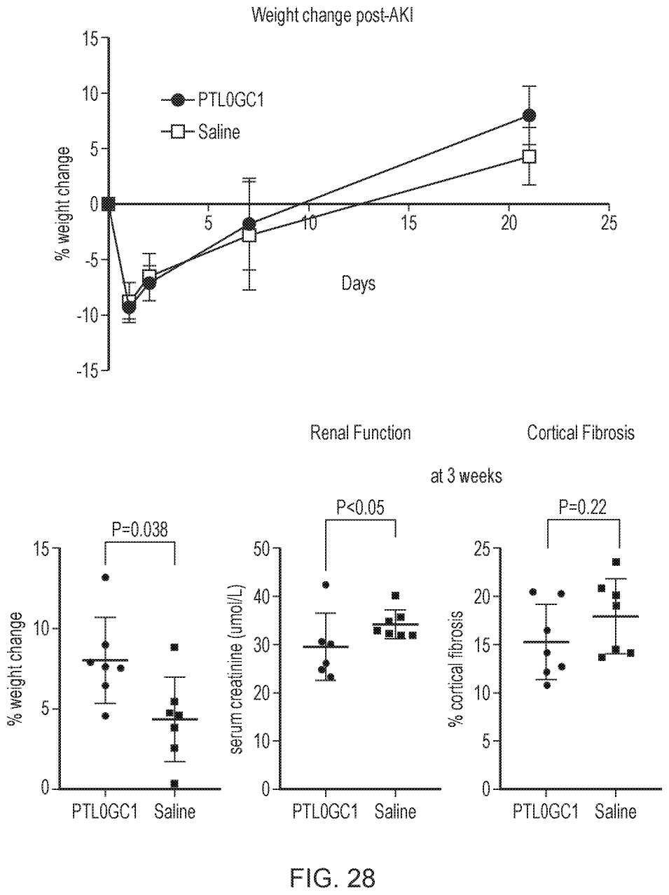

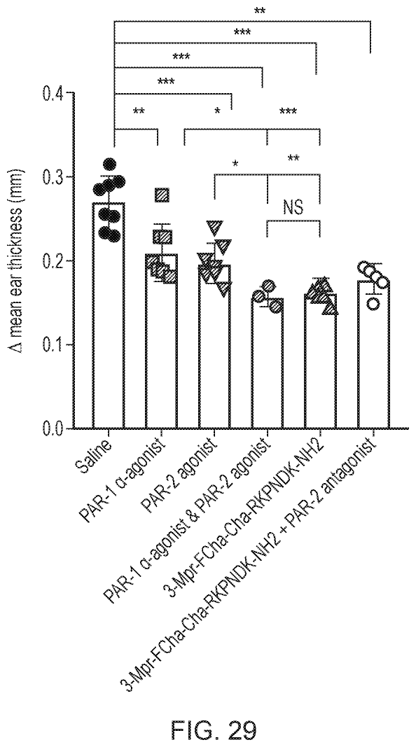

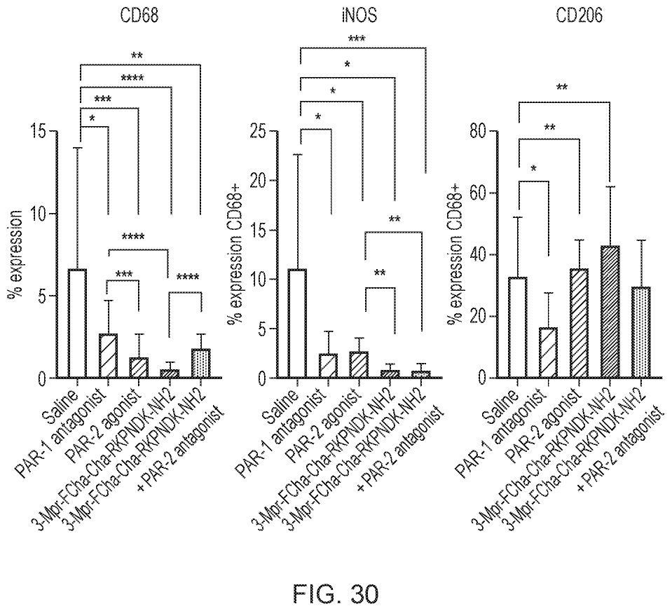

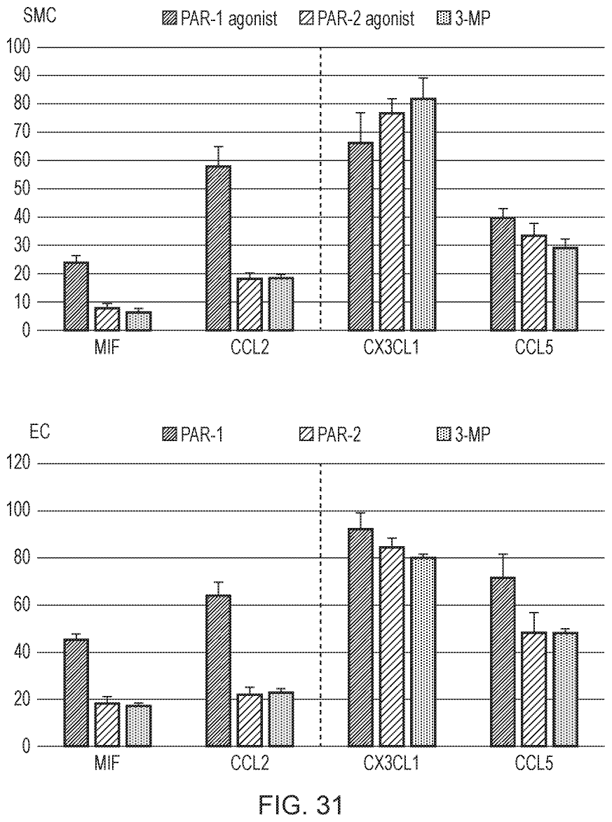

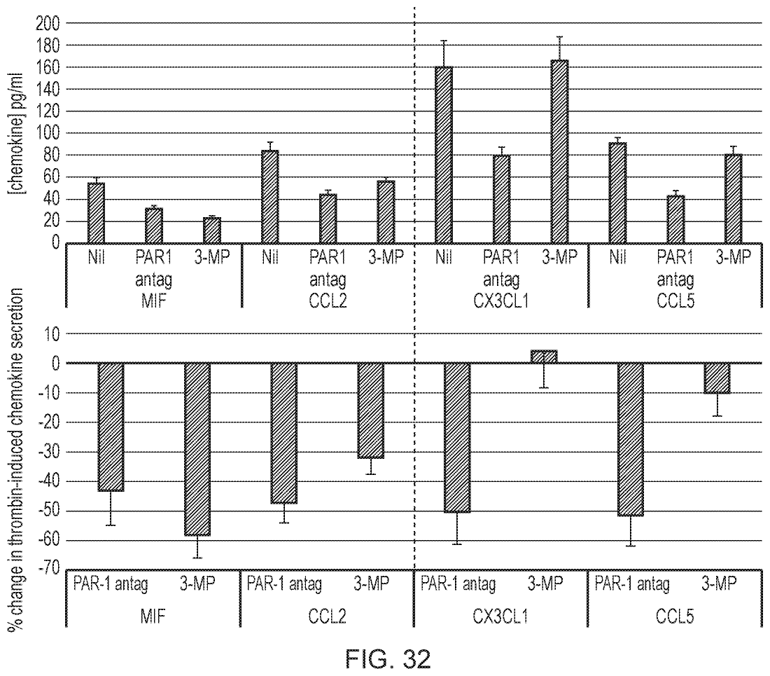

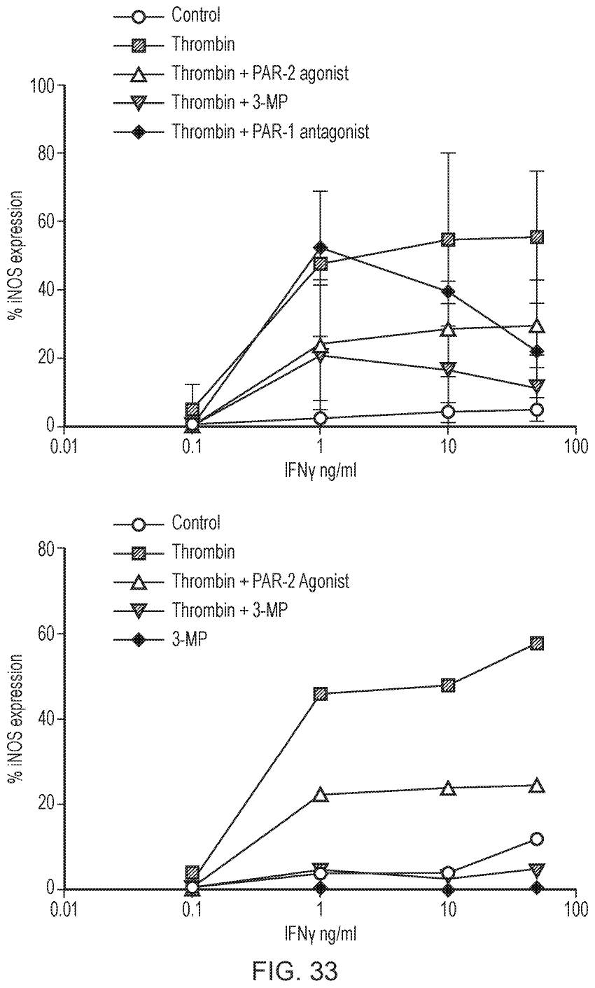

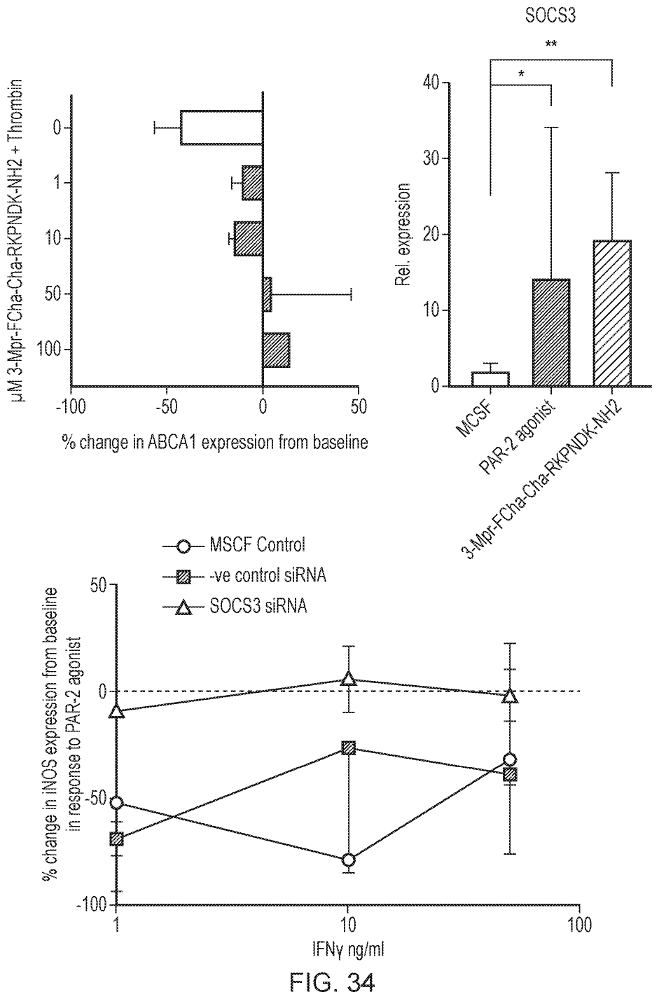

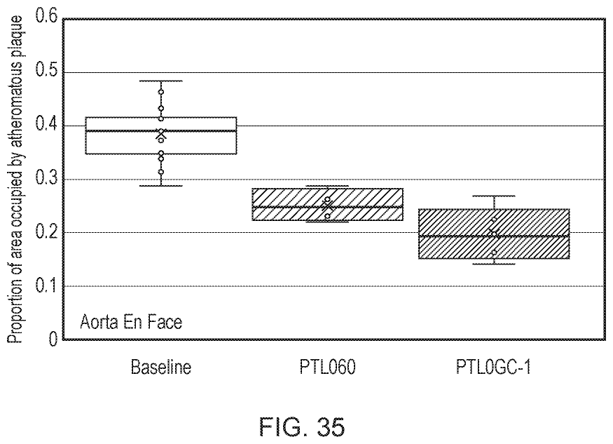

FIG. 9 shows supplementary FIG. 1 which shows a diagram

FIG. 10 shows supplementary FIG. 2 which shows photographs: Inhibition of TF or thrombin on EC abolishes CCL2 and MIF expression in vascular walls

A-C. Three colour immunofluorescence images of sections through donor aortas, 6 weeks post-transplantation. Recipients were ApoE−/− mice, fed a high fat diet (HFD) for two weeks from age 6 weeks, prior to transplantation of aorta from BL/6 (A) CD31-TFPI-Tg (B) or CD31-Hir-Tg (C). Blue—nuclear stain 4′,6-diamidino-2-phenylindole (DAPI). Red—anti-CD31 (A) anti-hTFPI (B) or anti-hirudin (Hir-C). Green—CCL2. Each panel of three images shows consecutive sections.

D&E: Three colour IF images of consecutive sections through aortic root, taken 1, 2 or 3 weeks post IV injection of 10 mg/g of PTL060 (D) or PBS (E). ApoE−/− mice were commenced on a high fat diet 2 weeks prior to the injections. Blue—DAPI. Red—anti-CD31. Green—MIF.

FIG. 11 shows supplementary FIG. 3 which shows a bar chart: PTL060 inhibits thrombin- and PAR-1-mediated chemokine production in vitro.

In vitro analysis of MIF (A) or CCL2 (B) production by cultured mouse SMCs, following stimulation by thrombin, with addition of reagents to demonstrate that PTL060 predominantly inhibits PAR-1 mediated chemokine production.

FIG. 12 shows supplementary FIG. 4 which shows bar charts: Systemic inhibition of inflammation by PTL060.

Graphs show box plots with median with interquartile range (IQR) with whiskers showing upper and lower limits and outliers indicated as single data points. Means are represented with ‘x’. P values by Mann Whitney U test.
Plasma TNFα (A), IFNg (B), MIF (C) and CCL2 (D) in different groups of ApoE−/− mice, as indicated on abscissa.

FIG. 13 shows supplementary FIG. 5 which shows photographs and bar charts: Phenotype of plaque cells induced by PTL060_2

Confocal microscopic analysis of three colour immunofluorescence images through consecutive sections of aortic roots of ApoE−/− mice, fed a high fat diet from 6 to 22 weeks (‘Baseline’, all panels) or 6-28 weeks, with mice administered weekly injections of saline, HLL, or PTL060 as indicated between weeks 22-28. Panels show the plaque expression of CD68 (red) with (green) either IL-10 (A) IFNγ (E) or TNFα (I). Yellow in overlay image indicates co-localisation. The plaque area is demarcated by the lumen (L) and the dotted white line. Le=aortic leaflet.
Each panel of images is accompanied by graphical representations of the % of plaque area staining for the molecule of interest (B-IL-10, F-IFNγ, J-TNF α) and the % of plaque area occupied by CD68+ (C, G, K) and the proportion of CD68+ cells (white bars) and CD68-negative cells (grey bars) co-staining for IL-10 (D), IFNγ (H), or TNF α (L). Each graph is a box plot with median with interquartile range (IQR) with whiskers showing upper and lower limits and outliers indicated as single data points. Means are represented with ‘x’. Each is derived from an assessment of each of the three aortic root plaques from at least 6 individual mice.

FIG. 14 shows supplementary FIG. 6 which shows photographs and bar charts: Phenotype of plaque cells induced by PTL060_3

Confocal microscopic analysis of three colour immunofluorescence images through consecutive sections of aortic roots of ApoE−/− mice, fed a high fat diet from 6 to 22 weeks (‘Baseline’, all panels) or 6-28 weeks, with mice administered weekly injections of saline, HLL, or PTL060 as indicated between weeks 22-28. Panels show the plaque expression of CD68 (red) with (green) either iNOs (A) or CD206 (E). Yellow in overlay image indicates co-localisation. The plaque area is demarcated by the lumen (L) and the dotted white line. Le=aortic leaflet.
Each panel of images is accompanied by graphical representations of the % of plaque area staining for the molecule of interest (B-iNOS, F-CD206) and the % of plaque area occupied by CD68+ (C, G) and the proportion of CD68+ cells (white bars) and CD68-negative cells (grey bars) co-staining for iNOS (D) or CD206 (H). Each graph is a box plot with median with interquartile range (IQR) with whiskers showing upper and lower limits and outliers indicated as single data points. Means are represented with ‘x’. Each is derived from an assessment of each of the three aortic root plaques from at least 6 individual mice.

FIG. 15 shows a bar chart.

FIG. 16 shows molecular structures.

FIG. 17 shows plots.

FIG. 18 shows bar chart.

FIG. 19 shows bar chart.

FIG. 20 shows bar chart.

FIG. 21 shows plots.

FIG. 22 shows bar charts.

FIG. 23 shows bar charts.

FIG. 24 shows supplementary FIG. 7 which shows photographs and bar charts:

Impact of adoptive transfer of CD11b+ cells expressing hirudin_2
All panels: CD11b cells, harvested from either BL/6 or CD31-Hir-Tg mice were labelled in vitro with PKH26 (red) and adoptively transferred into ApoE−/− mice fed a HFD between ages of 6-22 weeks. Aortic roots were collected 48 hours post-injection, for confocal IF analysis of the phenotype of adoptively transferred cells. Graphs are a box plot with median with interquartile range (IQR) with whiskers showing upper and lower limits and outliers indicated as single data points. Means are represented with ‘x’. Each is derived from a double assessment of each of the six aortic root plaques from 6 individual mice.
A-C: To illustrate expression of IFNγ (green) within the plaque after adoptive transfer of CD11b+ cells from BL/6 (A) or CD31-HIr-Tg (B) mice. (C) illustrates quantitative assessment of the proportion of PKH26+ cells co-expressing IFNγ.
D-F: To illustrate expression of IL-10 (green) within the plaque after adoptive transfer of CD11b+ cells from BL/6 (D) or CD31-HIr-Tg (E) mice. (F) illustrates quantitative assessment of the proportion of PKH26+ cells co-expressing IL-10.
G-I: To illustrate expression of iNOS (green) within the plaque after adoptive transfer of CD11b+ cells from BL/6 (G) or CD31-HIr-Tg (H) mice. (I) illustrates quantitative assessment of the proportion of PKH26+ cells co-expressing iNOS.
J-L: To illustrate expression of CD206 (green) within the plaque after adoptive transfer of CD11b+ cells from BL/6 (J) or CD31-HIr-Tg (K) mice. (L) illustrates quantitative assessment of the proportion of PKH26+ cells co-expressing CD206.

FIG. 25 shows supplementary FIG. 8 which shows photographs and bar charts:

Monocyte recruitment and phenotype after systemic PTL060_2.
Confocal microscopic analysis of three colour immunofluorescence images through consecutive sections of aortic roots of ApoE−/− mice, fed a high fat diet from 6 to 26 weeks, with mice administered weekly injections of saline or PTL060 as indicated below between weeks 22-25. 1 week after the last injection, mice were injected with PKH2-labelled CD11b cells (green) and aortic roots harvested 48 hours later. Graphs are a box plot with median with interquartile range (IQR) with whiskers showing upper and lower limits and outliers indicated as single data points. Means are represented with ‘x’. Each is derived from a double assessment of each of the three aortic root plaques from 3 individual mice.
A-C: To illustrate the expression of IL-10 (red) after adoptive transfer of BL/6 CD11b+ cells in mice treated with saline (A) or PTL060 (B). (C) illustrates quantitative assessment of the proportion of PKH2+ cells co-expressing IL-10.
D-F: To illustrate the expression of TNFα (red) after adoptive transfer of BL/6 CD11b+ cells in mice treated with saline (D) or PTL060 (E). (F) illustrates quantitative assessment of the proportion of PKH2+ cells co-expressing TNFα.
G-I: To illustrate the expression of IFNγ (red) after adoptive transfer of BL/6 CD11b+ cells in mice treated with saline (G) or PTL060 (H). (I) illustrates quantitative assessment of the proportion of PKH2+ cells co-expressing IFNγ.

FIG. 26 shows bar charts. In more detail, this shows Model 1: Atherosclerosis: Superior regression induced by PTL0GC-1 compared to both PTL060 and untailed 3-mercaptopropionyl-F-Cha-Cha-RKPNDK. ApoE−/− mice were fed a high fat diet for 16 weeks (baseline), prior to weekly IV treatment for 6 weeks with either saline, PTL060 10 mg/g, equimolar PTL0GC-1 or equimolar *3-MP. Extent of atherosclerosis was explored by en face examination of the surface area of aorta containing plaques (top panel) or after determination of the proportion of cross-sectional area through the aortic root occupied by plaque, using Oli-Red-O staining. *3-MP=3-mercaptopropionyl-F-Cha-Cha-RKPNDK

FIG. 27 shows a diagram, a plot and a bar chart. In more detail, Model 2: Delayed type hypersensitivity: Anti-inflammatory effect of PTL0GC-1 is superior to that of PTL060 and untailed 3-mercaptopropionyl-F-Cha-Cha-RKPNDK. C57BL/6 mice were topically sensitised with oxazolone on the shaved abdomen on day 0. Treatment with either saline, PTL060 (10 mg/g), equimolar PTL0GC-1 or equimolar *3-MP was given on day 3 and day 5, the last immediately prior to re-challenge with oxazolone onto the right ear. The left ear was treated with vehicle alone. On day 6, the difference in swelling of the right and left ears was measured and is presented as D mean ear thickness. PTL0GC-1 intravenously has the biggest effect, but this data suggests efficacy of both IP and SC administration. *3-MP=3-mercaptopropionyl-F-Cha-Cha-RKPNDK

FIG. 28 shows graphs and plots. In more detail, Model 3: Ischaemia reperfusion injury: PTL0GC-1 induces quicker recovery, associated with reduced fibrosis, post acute kidney injury. Both kidneys of C57BL/6 mice were exposed under recovery anaesthesia, and both renal arteries completely occluded by clamps for 30 minutes, prior to release. 2 doses of PTL0GC-1 (7.5 mg/ml) or saline were administered to the mice immediately prior to clamping, and immediately prior to clamp release. Mice were allowed to recover and then monitored for 3 weeks, prior to euthanasia. PTL0GC-1 has no impact on the AKI, but during recovery phase, mice gain weight quicker and achieve better renal function by 3 weeks. Histological analysis of the kidneys reveals a trend towards less fibrosis developing in the PTL0GC-1-treated mice.

FIG. 29 shows a bar chart. In more detail, delayed type hypersensitivity: confirmation that the anti-inflammatory effect of 3-mercaptopropionyl-F-Cha-Cha-RKPNDK is due to antagonism of PAR-1 signalling combined with agonist activity at PAR-2. PAR-1 inhibition combined with PAR-2 activation best inhibits ear swelling. Pure PAR-1 antagonist (αagonist)=FLLRN; Pure PAR-2 agonist=2-Furoyl-LIGRLO-amide; Pure PAR-2 antagonist=FSLLRY-NH2 trifluoroacetate salt; All used at 10 microM/g. *P≤0.05, ** P≤0.01, ***P≤0.001, **** P≤0.0001

FIG. 30 shows bar charts. In more detail, delayed type hypersensitivity: confirmation that the anti-inflammatory effect of 3-mercaptopropionyl-F-Cha-Cha-RKPNDK is associated with reduced monocyte recruitment with switched phenotype. PAR-1 inhibition combined with PAR-2 activation best inhibits CD68+ macrophage infiltration during delayed type hypersensitivity. Infiltrating macrophages are skewed away from an iNOS+‘M1’ phenotype towards a CD206+‘M2’ phenotype. Pure PAR-1 antagonist (αagonist)=FLLRN; Pure PAR-2 agonist=2-Furoyl-LIGRLO-amide; Pure PAR-2 antagonist=FSLLRY-NH2 trifluoroacetate salt; All used at 10 microM/g. *P≤0.05, ** P≤0.01, ***P≤0.001, **** P≤0.0001

FIG. 31 shows bar charts. Y-axis shows [chemokine] pg/ml. In more detail, Mechanistic insights—monocyte recruitment: In vitro secretion of chemokines by PAR-1 and PAR-2 agonists. Mouse primary smooth muscle cells (SMCs) or endothelial cells (EC) at a density of 1×10⁶ cells/well of a 24-well plate were serum-starved for 24 hours before addition of FLLRN (PAR-1 antagonist, 10 μM), 2-Furoyl-LIGRLO-Amide (PAR-2 agonist, 10 μM) or 3-MP (3-mercaptopropionyl-F-Cha-Cha-RKPNDK 10 μM) for 12 hours, following which thrombin (10 nM) was added for 1 hour, before a final 48 hours incubation in 2% FCS DMEM. Supernatants of stimulated cells were collected to measure MIF, CCL2, CCL5 and CX3CL1 using ELISA, following manufacturer's protocol.

### Interpretation:

MIF and CCL2 are associated with recruitment of CCR2+ (Ly6Chi) monocytes CX3CL1 and CCL5 associated with recruitment of CCR2− (Ly6Clo) monocytes
The two chemokines on the left of the panel (MIF, CCL2) are secreted after stimulation by a PAR-1 agonist, but less so by a PAR-2 agonist.
In contrast, those on the right of the panel (CX3CL1 and CCL5) are secreted after stimulation by either PAR-1 or -2 agonists.
3-MP in this assay behaves as a pure PAR-2 agonist.

SMC and EC respond equivalently

FIG. 32 shows bar charts. In more detail, mechanistic insights—monocyte recruitment: In vitro secretion of chemokines by thrombin—differential impact of PAR-1 antagonist and 3-mercaptopropionyl-F-Cha-Cha-RKPNDK. Secretion of chemokines from murine SMC after stimulation with thrombin alone (nil), or in presence of a pure PAR-1 antagonist, or 3-MP*. Top panel shows the actual concentration of chemokine secreted. Bottom panel shows the % change in thrombin induced chemokine secretion by the PAR-1 antagonist and 3-MP

Thrombin-induced secretion of MIF and CCL2 are reduced by 30-50% by both a PAR-1 antagonist and 3-MP
In contrast, thrombin-induced secretion of CX3CL1 and CCL5 are reduced by 50% by the PAR-1 antagonist, but by only 0-10% by 3-MP
*3-MP=3-mercaptopropionyl-F-Cha-Cha-RKPNDK

FIG. 33 shows graphs. In more detail, mechanistic insights: direct effect on monocyte/macrophage phenotype. Sensitivity of macrophages to IFNγ, as assessed by the proportion that express iNOS after stimulation.

The enhanced sensitivity to IFNγ induced by thrombin is inhibited by a PAR-1 antagonist—this is via expression of ABCA1 and membrane lipid rafts.
A pure PAR-2 agonist also reduces the sensitivity of macrophages to IFNγ induced by thrombin.
3-MP completely (bottom) or near completely (top) abolishes the effect of thrombin on sensitivity to IFNγ. It also inhibits the background response of control macrophages to IFNγ.
BMM purified cells cultured in MCSF for 5 days. Cells then removed for 24 hours and incubated with thrombin (50 units) (if an antagonist or 3-mercaptopropionyl-F-Cha-Cha-RKPNDK was used cells were incubated for 2 hours with the antagonist or 3MP prior to thrombin stimulation to allow time for antagonist to dock with PAR). Cells then had media replaced with thrombin+ PAR reagent and IFN gamma at the dose described. For the PAR2 experiments it was at this point that PAR2 was added. Cells were then incubated for 24 further hours and then analysed by flow cytometry for iNOS. Pure PAR-1 antagonist (αagonist)=FLLRN; Pure PAR-2 agonist=2-Furoyl-LIGRLO-amide; 3-MP=3-mercaptopropionyl-F-Cha-Cha-RKPNDK; All used at 100 microM. Thrombin used at 50 iu

FIG. 34 shows bar charts and a graph. In more detail, mechanistic insights: direct effect on monocyte/macrophage phenotype through SOCS3. To explain how 3-MP inhibits the enhanced sensitivity to IFNγ induced by thrombin. As shown in top left graph, 3-MP, titrated in during incubation of macrophages with thrombin, inhibits thrombin-induced ABCA1 expression, as shown for a PAR-1 antagonist (published), but does not enhance ABCA1 expression. Therefore, the superior effect of 3-MP compared to a pure PAR-1 antagonist is not explained by action on ABCA1

A pure PAR-2 agonist induces SOCS3 expression, as does 3-MP (middle graph). This is a well-described inhibitor of IFNγ signalling.
In the right graph, the thrombin-induced sensitivity to IFNγ, as assessed by change in iNOS expression, is shown. Incubation with a PAR-2 agonist dampens the enhanced sensitivity. A control siRNA does not inhibit the effect of the PAR-2 agonist. However, an siRNA against SOCS3, to inhibit SOCS3 expression, abolishes the effect of the PAR-2 agonist, suggesting that PAR-2 signalling works through SOCS3 upregulation.

FIG. 35 shows a bar chart. In more detail, mechanistic insights: direct effect on monocyte/macrophage phenotype. ApoE−/− mice were fed a high fat diet for 16 weeks (baseline), prior to weekly IV treatment for 6 weeks with monocytes from BL6 mice, pre-treated for 30 minutes with either PTL060 (100 microM) or PTL0GC-1 (actually used at ¼ concentration-25 microM).

Extent of atherosclerosis was explored by en face examination of the surface area of aorta containing plaques using Oil-Red-O

### Interpretation:

Treatment of mice with adoptively transferred monocytes only, after incubation with either PTL060 or PTL0GC-1, is sufficient to induce the same degree of regression associated with IV treatment of the same reagents, suggesting that a direct effect on monocytes may be the dominant mechanism of action.

### EXAMPLES

### Example 1—PTL060: A Cytotopic Direct Thrombin Inhibitor

In this example, PTL060, a cytotopic direct thrombin inhibitor, exemplifies the point that cytotopic modification uncouples the anti-inflammatory effect from systemic anticoagulation during regression of atheroma.

Our experience with the cytotopic compound PTL060, which is a tailed version of the direct thrombin inhibitor bivalirudin, is that the cytotopic tailing process endows bivalirudin with prolonged biological activity, such that weekly (or less frequent) dosing of the tailed moiety is sufficient for therapeutic activity (see FIG. 15).

FIG. 15 shows the impact of cytotopic modification. Percentage of aortic root on cross sectional analysis occupied by atheroma as assessed by Oil Red O staining. Graph shows box plots of median with interquartile range (IQR) with whiskers showing upper and lower limits of % area in the whole group (N=6 per group). All were ApoE−/− mice fed a HFD for 16 weeks. ‘Baseline’ group was assessed at this point. The remaining two groups then received weekly IV injections of either saline (‘Control’), PTL060 (10 mg/g) or an equimolar dose of parental untailed anticoagulant (bivalirudin) for 6 further weeks, whilst remaining on a HFD. Comparing PTL060: to baseline, p=0.03: to bivalirudin p=0.001. (Mann Whitney test).

Using ApoE−/− mice that were fed a high fat diet (HFD) for 16 weeks, IV injection of PTL060 (10 μg/g) weekly for six weeks induced regression in the burden of atheromatous lesions present at the aortic root. The lesional burden reduced by >40% compared to control mice receiving saline injection, but importantly, also reduced by >35% compared to baseline mice examined at week 16, prior to beginning any PTL060 injections. In stark contrast, the mice receiving equimolar doses of parental bivalirudin on the same dosing schedule developed increasing atheroma burden equivalent to that seen in saline control mice. Interestingly, these effects were seen without reductions in plasma total cholesterol levels.

Most importantly, mice treated with PTL060 were systemically anticoagulated for only 1/7^{th} of the time between doses (see example below). Thus, the addition of the cytotopic tail to bivalirudin uncouples the pharmacodynamics of its impact on haemostasis from its effects on inflammation, at doses that both prevent plaque formation and induce plaque regression. To our knowledge, this is the first demonstration of such uncoupling, and represents a significant advance in understanding the true therapeutic potential of targeting coagulation proteases to influence inflammatory disease.

In this model, PTL060 works via two distinct mechanisms (see example below). First, it acts at the vascular wall to promote the recruitment of predominantly CCR2-Ly6Clo monocytes, which are known to be precursors of M2 polarised macrophages. These recruits express CD206, IL-10, ABCA1 and CCR7 and have a phenotype previously associated with regression.

Second, it acts directly on circulating monocytes, and protects them from the effects of thrombin signalling through PAR-1. This means that upon recruitment into the plaque, all monocytes adopt a ‘regression’ phenotype (see example below).

### Example 2—Regression of Atherosclerosis in Apoe−/− Mice Via Modulation of Monocyte Recruitment and Phenotype, Induced by Weekly Dosing of a PTL060 ‘Cytotopic’ Anti-Thrombin without Prolonged Anticoagulation

Background: Coagulation proteases play an important role in atherogenesis. Accordingly, anticoagulants can induce regression in animal models of atherosclerosis, but exploiting this clinically has been limited by major bleeding events that occur after systemic anticoagulation. Here we test a novel thrombin inhibitor, PTL060, that comprises hirulog covalently linked to a synthetic myristoyl electrostatic switch to tether it to cell membranes.

Methods: ApoE−/− mice, fed either chow or high fat diets were used. Transplantation of congenic aortic segments was used to demonstrate the impact of expressing anticoagulants on endothelium. PTL060, parental hirulog or controls were tested to assess suppression of vessel wall chemokine gradients, impact on plaque development and regression of existing plaques. Adoptive transfer of labelled CD11 b positive cells was used to assess recruitment of monocytes and inform on how PTL060 influenced monocyte phenotype.

Results: Transgenic expression of anticoagulant fusion proteins based on TFPI or hirudin on EC led to complete suppression of MIF and CCL2 expression throughout the vessel wall and segments of aorta transplanted into ApoE−/− mice did not develop atherosclerosis. A single IV injection of PTL060, but not parental (unmanipulated) hirulog inhibited the same chemokines for >1 week and atheroma formation was reduced by >50% compared to controls when assessed 4 weeks later. Mice had prolonged bleeding times for only 1/7^{th} of the time that PTL060 was biologically active. Repeated weekly injections of PTL060 but not parental hirulog caused regression of atheroma in ApoE−/− mice fed either chow or high fat diets. Mechanistically, 100% of circulating monocytes quickly became coated with PTL060 after the first dose, following which >70% of CCR2+ monocytes recruited into plaques expressed CCR7, ABCA1 and IL-10, a phenotype associated with regression, compared to <20% of CCR2+ recruits in control mice. Multiple doses caused a significant reduction in the number of monocytes recruited, and a switch to recruitment of CCR2-negative cells, the majority of which (>90%) had a similar regression-associated phenotype. The impact of PTL060 on circulating monocytes appeared dominant, as regression equivalent to that induced by IV PTL060 was induced by adoptive transfer of CD11b+ cells pre-coated with PTL060.

Conclusions: PTL060, a novel tethered direct thrombin inhibitor causes regression of atherosclerosis in ApoE−/− mice, via an effect at the endothelial surface but also through a direct effect on monocytes, causing differentiation into macrophages capable of plaque regression. Covalent linkage of a myristoyl electrostatic switch onto hirulog uncouples the pharmacodynamic effects on haemostasis and atherosclerosis, such that regression is accompanied by only transient anticoagulation.

### INTRODUCTION

Atherosclerosis, is a chronic inflammatory disease that causes coronary artery, peripheral vascular and cerebrovascular disease. It is a major cause of death in the Western world. Important early steps in atherogenesis, in the context of a high lipid microenvironment include secretion of chemokines such as CCL-2 and macrophage migration inhibitory factor (MIF)¹, by activated endothelial cells (ECs) and smooth muscle cells (SMCs)^{2,3}. These promote infiltration of monocytes into the subendothelial space, where they become macrophages and take up very low-density lipoprotein (VLDL) and low-density lipoprotein (LDL) to become foam cells, initiating the process of atheroma formation.

Coagulation proteases, such as thrombin, signal though protease activated receptors (PAR) as well as catalysing fibrin formation and are known to play a role in this process. Increased activity of tissue factor (TF), the 47-Kd cell membrane-bound glycoprotein that initiates the serine protease cascade, is seen in the neointima and underlying media of atherosclerotic plaques⁴⁻⁶ and TF is expressed by EC⁷, monocytes/macrophages⁸ and SMC⁹.

In previous work, we crossed a strain of transgenic mice expressing a membrane-tethered human Tissue Factor Pathway Inhibitor (hTFPI) fusion protein on α-smooth muscle actin (SMA)⁺ cells (called α-TFPI-Tg mice)¹⁰ with Apolipoprotein E-deficient (ApoE^{-/-}) mice to generate a new strain (called ApX4). These mice were resistant to atheroma formation¹¹. In dissecting the mechanism of resistance, we showed that TF expression by SMC was necessary to generate MIF, via generation of thrombin and signalling primarily through PAR-1. Inhibition of either the TF, thrombin mediated PAR-1 signalling or MIF secretion prevented atherosclerosis in mice fed either a high fat diet (HFD) or a regular chow-based diet.

One of the observations from that study was that MIF continued to be secreted by EC in ApX4 mice fed a HFD, beneath which small atherosclerotic plaques developed¹¹. This suggested that targeting SMC with hTFPI was not completely efficient at inhibiting atheroma. In this new study, we explored how transgenically-expressed tethered anticoagulants on EC impacted on atherosclerosis development, and assessed the translational potential of a novel thrombin inhibitor containing the potent peptide hirulog (a direct thrombin inhibitor), that has been chemically modified (HLL) to accept a lipid membrane-binding anchor or ‘cytotopic’ tail. This new compound is called PTL060 (thrombalexin-3). PTL060 has previously been localised within organs before transplantation to successfully inhibit thrombosis and rejection in several models¹²⁻¹⁴. In the process we describe an unpredicted impact of PTL060 on the phenotype of monocytes recruited into atherosclerotic plaques, by two interrelated pathways, one of which occurs by virtue of its ability to tether directly to monocytes. We also provide a mechanistic insight into the role that thrombin appears to play in driving plaque progression, as evidenced by the regression seen when PTL060 is administered systemically.

### Methods

### Mice and In Vivo Procedures

C57BL/6J (BL/6) mice were purchased from Harlan UK Ltd (Bicester, UK) and ApoE−/− mice were from the Jackson Laboratory (Bar Harbor, Me. 04609, USA). CD31-TFPI-Tg and CD31-Hir-Tg¹⁵ mice were bred in house. Mice were housed in a temperature-controlled Specific Pathogen-Free environment at 22-24° C. and all animal surgical protocols, animal experiments and care were approved by the UK Home Office. To assess the distribution of PTL060, male BL/6 mice weighing 25-28 g (n=6 per group) were injected IV through a tail vein with either PTL060 (10 μg/g in 100 μl saline), equimolar HLL (5 μg/g) or saline. At 5, 30 minutes and 2, 4, 6, 24 and 48 hours mice were sacrificed to collect citrated whole blood for separation into cells and plasma and to harvest aortas for immunofluorescence analysis.

Bleeding times were assessed as previously described¹⁵. Briefly, mice were anesthetized and placed in a restrainer (Becton Dickinson), before a distal 3-mm segment of tail was severed with a razor blade. The tail was immediately immersed in 0.9% saline at 37° C. Bleeding time was defined as the time required for bleeding to stop. Experiments were terminated at this time or at 20 minutes.

For all atherosclerosis work, male ApoE−/− mice, from the age of 6-weeks, were fed with a high fat diet (HFD) consisting 35 kcal % fat, 1.25% cholesterol, and 0.5% cholic acid (Special Diet Services, Essex UK). Aortas were transplanted 2 weeks after starting the HFD, using a sleeve anastomosis technique described previously¹¹. Briefly, a 5 mm of the segment of the infrarenal donor aorta, flushed with 300 μl of saline containing 50 U of heparin, was transplanted into ApoE−/− recipient abdomen aortas (N=6 per group). Blood flow was confirmed by direct inspection after removal of the clamps. Mice were fed a HFD for 6-12 weeks post-transplantation before the experiment was terminated (Suppl FIG. 1A). To assess prevention of atheroma, mice (n=6 per group) received a single injection of PTL060 or controls by tail vein, and the experiments terminated 1-3 weeks later (Suppl FIG. 1B). For regression experiments, baseline groups (n=6) were fed a HFD to the age of 22 weeks before the mice were sacrificed. Experimental or control groups (n=3-6 per group) received weekly injections by tail vein for 3-6 weeks, beginning at age of 22 weeks before the experiments were terminated when mice were 25-28 weeks old (Suppl FIG. 1C).

### Cell Isolation and Labelling.

Leukocytes were isolated from the blood of mice aged 8-10 weeks, using anti-CD11b MicroBeads (Miltenyi Biotec Ltd, Surrey, UK) according to manufacturers' instructions. For cell labelling, 2×10⁷ CD11b+ cells were incubated with 4×10⁻⁶ M of either PKH26 or PKH2 fluorescent dyes (Sigma, UK) for 5 minutes at 25° C. according to manufacturers' protocols, with the reaction stopped using 1% BSA in PBS followed by three washes. Each recipient mouse received 0.5×10⁶ cells by IV injection; in some experiments, the cells were incubated with PTL060 (100 μM in 0.5 mls) or equimolar controls for 30 minutes at room temperature and washed three times before immediate injection. For viability assays, murine bone marrow cells were incubated for 5 days in 6 well plates, counted and re-seeded at 2×10⁵ cells/ml in 24 well plates with 25 ng/ml MCSF. After 1 day, media was replaced with new DMEM/FCS containing different concentrations of PTL060 (or a fixed volume of control PBS), and incubated for 30-120 minutes.

### Histological Analysis

Atherosclerotic lesions were evaluated as previously described¹¹. Simply, the entire length of the aorta was perfused with PBS, dissected using a dissecting microscope, longitudinally opened and stained with Oil Red O (ORO) solution (Sigma, UK) for 30 minutes, before being photographed with a digital camera (DSC-W320, Sony, Japan). The total aortic area and lesional area were measured by using Image J. Aortas from every animal were assessed. To assess lesions in the aortic sinus, hearts were embedded in paraffin, sectioned through the aortic root and incubated with elastin/van Gieson stain using the Accustain™ Elastin Stain kit (Sigma). Sections were examined on an Olympus U-ULH optical microscope (Olympus Optical Co. Ltd, Tokyo, Japan). Atheromatous lesional and total aortic root area was determined using Image-Pro Plus TM software version 4.0 (Media Cybernetics, Silver Spring, USA). At least three random sections were examined from each mouse in all groups.

Immunohistology of frozen cross sections were prepared and examined as previously described¹¹. Briefly, isolated tissues were snap-frozen and embedded in OCT compound (VWR International, Dorset, UK), sectioned at 5 μm thickness and fixed in methanol at −20° C. Frozen sections were immersed in 1% bovine serum albumin-phosphate-buffered saline (BSA-PBS) for 30 minutes and then incubated overnight at 4° C. with one or more of the following antibodies: rabbit polyclonal antibody to CD68, iNOs, CD206, TNFα, MIF, CCR7 ABCA1 (all from Abcam, Cambridge, UK), or hirudin (Biobyt, Cambridge, UK) or CCL2 (Lifespan BioScience, Inc., WA 98121, USA); goat polyclonal antibody to CD31 (Santa Cruz Biotechnology, Texas 75220, USA); rat anti-mouse CD68, CD11b (Serotec, Oxford, United Kingdom), CD31, IFNγ (BD Bioscience Pharmingen, Oxford, United Kingdom), Ly-6G (BioLegend, London, UK), IL-10 (Abcam) or biotinylated anti-HLL (RICS-2)¹⁴; mouse anti-CCR2 (Abcam). The following were used as isotype controls; goat anti-rat antibodies to IgG2a, IgG2b (BD Bioscience, Berkshire, UK) and polyclonal rabbit IgG (Abcam). The following anti-IgG FITC or TRITC-conjugated antibodies were used: sheep anti-mouse, rabbit anti-rat, goat anti-rabbit and rabbit anti-goat (all from Sigma). Fluorescein-conjugated streptavidin (Jackson Immunoresearch, Cambridge, UK) was used to detect RICS-2. Stained sections were mounted in Vectashield with DAPI (Vector Laboratories Inc, CA USA). Sections were directly captured and examined by a Leica DMIRBE confocal microscope (Leica, Wetzlar, Germany) equipped with Leica digital camera AG and a confocal laser scanning system with excitation lines at 405, 488, 543, and 560 nm at magnifications 10×/0.40 CS and 20×/0.70 IMM (Leica, Planapo, Wetzlar, Germany). Images were processed using Leica-TCS-NT software associated with the Leica confocal microscope. All immunohistochemistry was performed at 22° C. Quantification of staining was achieved by expressing the area of positive staining as a ratio of the total lesion area, calculated using Image-Pro Plus TM, version 4.0. All quantification was performed by members of the team blinded to the identity of the sections. For estimations of positive stained area, average measurements were derived from examination of at least six random sections from each tissue sample. To detect macrophage-derived foam cells, frozen sections of aortic sinus were analysed by a combination of ORO staining and CD68 immunostaining. Sections were incubated with rat anti-mouse CD68 antibody (overnight at 4° C.) and goat anti-rat antibody (1 hour at room temperature) before staining with filtered ORO solution (0.5% in propylene glycol, Sigma) for 15 minutes at room temperature.

### Plasma Assays.

Anticoagulated whole blood (EDTA 30 mM pH8) was separated into plasma and cells by centrifugation (14,000 g for 10 mins). Plasma TNFα, IFN-γ, MIF and CCL2 were detected using separate specific ELISA kits (R&D Systems, Abingdon, UK) according to the manufacturers' instructions. Total cholesterol, high-density lipoprotein and low-density lipoprotein were determined using kits from Cell Biolabs, and Tryglycerides with a kit from Abcam, (both Cambridge, UK) according to the manufacturer's protocol. Thrombin clotting times were measured in 3.2% trisodium citrated plasma according to the protocol of Ignjatovic¹⁶. Briefly, 100 μl mouse plasma was incubated with 2.5 U of human thrombin in a total volume of 300 μl (Enzyme Research Laboratories (ERL), Swansea, UK) at 37° C., and the time for a clot to form was measured (n=6 per group). For some experiments plasma was further centrifuged (20,000 g for 10 mins) to minimise the presence of extracellular vesicles.

### Flow Cytometry

The cells obtained from whole blood were washed twice in PBS with 2% FCS before staining with either anti-CD11b-FITC (Abcam) or anti-CD41-FITC (eBioscience) with biotinylated RICS2 followed by Streptavidin-PE (Bio-rad). Cells were then washed twice before analysis on a BD FACSCALIBUR with CellQuest Pro software.

Erythrocytes were identified by forward/side scatter profile.

For viability assays, cells were washed twice with PBS and then incubated with Fixable LIVE/DEAD Near-IR fluorescent reactive dye (Life Technologies) for 30 minutes at 4° C. Cells were washed, fixed for 15 minutes in 1% paraformaldehye, then washed with PBS-5% FCS and stored at 4° C. before acquisition and analysis within 24 hours on an LSRII/Fortessa flow cytometer at the BRC Flow Cytometry Laboratory, King's College London with Flowjo software (Treestar Inc). Macrophages identified by forward/side scatter profile.

### SMC-MIF/CCL2 Release Assay In Vitro

SMCs, cultured as previously described¹¹ and seeded at a density of 1×10⁶ cells/well of a 24-well plate were serum-starved for 24 hours before addition of PTL060 (100 μM) for 1 hour, followed by PAR agonists or antagonists (all from ERL) for 12 hours, followed by thrombin 10 nM or active site inhibited thrombin (ERL) for 48 hours, before collection of supernatants. Chemokines were measured by ELISA according to the manufacturers' instructions (R&D systems, Abingdon, UK)

### Statistical Analysis

Statistical analysis was performed with GraphPad Prism software.

### Results.

### Anticoagulants Transgenically Localised to EC Completely Inhibit Vessel Wall Expression of Chemokines and Prevent Formation of Atheroma.

To assess the impact of expressing hTFPI fusion protein on EC alone, we used the congenic aortic transplant model previously described¹¹, and compared the extent of atheroma development in transplanted aortas from CD31-TFPI-Tg mice (expressing hTFPI transgene on EC¹⁵) and BL/6 mice. The recipients were 8-week old ApoE−/− mice fed a HFD for 2 weeks prior to the transplant, and the experiment was terminated 6 weeks after the transplant (suppl FIG. 1). In the aortic transplants from CD31-TFPI-Tg mice, MIF expression was absent through the entire wall of the transplanted vessel, not just the EC (FIG. 1 A&B) and atheroma formation was significantly attenuated in the transplanted donor segment (FIG. 1 F&H). In contrast and as previously reported, control BL/6 aortic transplants developed exaggerated lesions, associated with MIF expression in all layers of the vascular wall (FIG. 1 E, G, H &J). The atherosclerosis that developed in the recipient aortas was independent of the type of donor aorta transplanted (FIG. 1 G, H &l).

Next we transplanted aortas from a second transgenic strain (CD31-Hir-Tg)¹⁵, expressing a tethered hirudin fusion protein on EC (suppl FIG. 1), and these showed similar suppression of MIF expression throughout the vessel wall and were similarly resistant to atheroma development (NB in this series of experiments, the time that aortas were left in situ was extended to 12 weeks post-transplantation.) (FIG. 1C, D, K-O), indicating that inhibition of thrombin and TF on EC was functionally equivalent, entirely consistent with our previously published results¹¹. In addition to MIF, CCL-2 expression was completely suppressed throughout the vessel walls of transplants from both transgenic strains (suppl FIG. 2). This data indicates that inhibiting thrombin generation (by TFPI) or the enzymatic activity of thrombin (by hirudin) on transgenic EC after transplantation into ApoE−/− mice completely suppresses MIF and CCL-2 expression throughout the entire vascular wall and prevents atheroma formation.

### IV Injection of PTL060, a Novel Tethered Therapeutic Anti-Thrombin.

After IV administration of PTL060 into BL/6 mice, linear deposition of the anticoagulant moiety could be found on the luminal surface of the aorta mice several hours later (FIG. 2A). This pattern of staining was never seen after injection of parental (untailed) HLL (FIG. 2B). PTL060 also very quickly attached to the lipid membranes of circulating erythrocytes, leukocytes and platelets (FIG. 2C-E), maintaining stable levels of binding between 2-6 hours post-injection, before reducing between 24-48 hours post injection. No binding was ever seen after injection of HLL. In vitro viability assays, performed by incubating BM-derived macrophages with increasing concentrations of PTL060 for 30-120 minutes confirmed no evidence of toxicity (suppl table 1). Supplementary Table 1

| | SUPPLEMENTARY TABLE 1 | | |
|---|---|---|---|
| | Incubation time (minutes) | | |
| | 30 | 60 | 120 |
| Control PBS | — | — | 99 |
| 25 μM PTL060 | 99 | 95 | 96 |
| 50 μM PTL060 | 96 | 98 | 92 |
| 100 μM PTL060 | 97 | 87 | 80 |

% viability within Macrophage Forward Scatter/Side scatter gate, as assessed by LIVE/Dead aqua fluorescent dye. See methods for details

Thrombin clotting times of citrated plasma were prolonged for >6 hours post injection of PTL060, indicating the presence of a thrombin inhibitor, to a similar extent as was seen after injection of an equimolar amount of HLL (FIG. 2F&G). This was associated with prolonged tail bleeding times (FIG. 2H) lasting for approximately 24 hours, with bleeding times in PTL060-treated mice very similar to those in mice given an equimolar concentration of HLL (FIG. 2H). Thus, IV injection of PTL060 resulted in rapid uptake onto the membranes of circulating cells and platelets, with detectable deposition on EC a few hours later. Despite this, the thrombin inhibitory activity detected in plasma was indistinguishable from that seen after injection of the HLL, and mice showed prolonged bleeding for 24 hours.

We assessed the differential impact of PTL060 and HLL on expression of MIF by the vasculature, as a biomarker of potential efficacy at suppressing atheroma formation. A single IV injection of PTL060 was accompanied by complete suppression of MIF expression throughout the vessel wall for almost 1-week (suppl FIG. 2D; FIG. 3A), which was dose dependent (FIG. 3D). This effect was never seen in controls given saline (suppl FIG. 2E) or in mice administered an equimolar dose of HLL (FIG. 3D). This prolonged biological effect of PTL060 is consistent with our previous demonstration that, once bound to endothelium, it remains detectable for >4 days¹⁴. In vitro experiments confirmed that thrombin-mediated chemokine expression was primarily via PAR-1 and that PTL060 completely inhibited this (suppl FIG. 3).

These data indicate that equimolar doses of PTL060 and HLL induce similar degrees of systemic thrombin inhibition lasting approximately 24 hours, but only PTL060 promotes prolonged suppression of MIF expression by vessel wall cells.

### Impact of PTL060 on Atherosclerosis

A single injection of PTL060 caused significant inhibition of atheroma formation in ApoE−/− mice fed a HFD for two weeks prior to, and four weeks after the injection (FIG. 3A, B&C). This effect was dose dependent (FIG. 3D, E-H), and only occurred with doses that inhibited MIF expression for up to 1 week (FIG. 3A, D; suppl FIG. 2D). It was not seen in mice administered equimolar doses of HLL (FIG. 3D, H). Thus, a membrane tethered thrombin inhibitor can replicate the impact of a transgenically expressed membrane tethered thrombin inhibitor by suppressing the development of atherosclerosis.

To assess the impact of PTL060 on established atheroma, 6-week old ApoE−/− mice were fed a HFD until the age of 22 weeks, before receiving IV injections of saline, HLL, control cytotopic tail compound or PTL060, weekly for a further 6 weeks. PTL060 caused a significant reduction in atheroma burden, when measured either by en face analysis or by cross sectional analysis of the aortic root (FIG. 4A-R), an effect not seen after weekly injections of any of the controls, including HLL at equimolar doses (FIG. 4 A-R). Atheroma burden was diminished even compared to baseline mice, analysed at week 16 prior to any treatment, indicating that disease regression was induced by PTL060. This was associated with a reduction in the area of plaque occupied by lipids, as shown by Oil-red O staining (FIG. 4S&U), as well as a reduction in the CD68+ cells co-localising with lipid (FIG. 4T&U), indicating a significant reduction in the number of foam cells within the plaques. All the effects of PTL060 were seen without any discernible impact on body mass or circulating lipid concentrations (table 1). PTL060 also significantly reduced atheroma burden after administration to ApoE−/− mice fed a normal chow diet, weekly from the age of 28 weeks for 6 weeks (FIG. 4V-Z). Under both HFD and Chow dietary conditions, administration of PTL060 was accompanied by significant reductions in plasma levels of TNFα, IFNγ, MIF and CCL2 (suppl FIG. 4), compared to the appropriate controls.

The Phenotype of Regressing Plaques after PTL060 Treatment

Atheromatous plaques in ApoE−/− mice fed a HFD from 6-22 weeks of age (baseline) contained a significant number of CD68+ cells (monocytes/macrophages), occupying approximately 45% of plaque area (FIG. 5A,F). Compared to control animals injected with either saline or HLL (FIG. 5B-G), weekly injections of PTL060 reduced the proportion of plaque area occupied by CD68+ cells to below 20% (FIG. 5D,F) with an associated increase in the proportion of plaque cells that were CD68-negative (FIG. 5G). The proportion of plaque area staining for MIF reduced from >60% at baseline (FIG. 5A, E) to <20% (FIG. 5 D,E) though the proportion of CD68+ and CD68-neg cells that expressed MIF was not altered (FIG. 5F,G). These data indicate that PTL060 induced a shift in plaque cell composition, from predominantly CD68+ cells in control mice to predominantly CD68-negative cells after PTL060 treatment, in association with a marked reduction in vessel wall MIF expression.

Plaques developing in mice fed a HFD between 6-22 weeks were almost devoid of cells expressing the chemokine receptor CCR7 (FIG. 5H,K) or the cholesterol efflux regulator ATP-binding cassette transporter molecule ABCA1 (FIG. 50,S), with <10% of plaque cells co-expressing these molecules (FIGS. 5I,J & T,U). Six weeks of treatment with weekly PTL060 from weeks 22-28 caused significant increases in the proportion of plaque area occupied by cells expressing CCR7 and ABCA1 (FIG. 5 H,N,R,S) and almost all of these were CD68+ cells (FIG. 5 I,N,R,T), compared to control saline-treated mice and in contrast to mice treated with weekly injections of HLL, in whom expression of both CCR7 and ABCA1 was similar to that seen at baseline (FIGS. 5H-M & O-U). In parallel, little IL-10 staining was seen within the plaques of any of the control animals (suppl FIG. 5A,B), and <2% of plaque-infiltrating CD68+ cells co-expressed IL-10 (suppl FIG. 5C), whereas almost all plaque infiltrating CD68+ cells expressed IFNγ (suppl FIG. 5E, G)) and TNFα (suppl FIG. 5I,K) and 15-20% of plaque CD68-negative cells expressed these two pro-inflammatory cytokines (supp FIG. 5H,L). After six weeks of PTL060, 10-15% of plaque area stained for IL-10 (suppl FIG. 5A, B), including 60% of the plaque-infiltrating CD68+ cells (suppl FIG. 5C), and approximately 10% of CD68-negative cells (suppl FIG. 5D). There was a marked reduction in both the proportion of plaque area (suppl FIG. 5E,F, I,J) and the proportion of CD68+ cells (suppl FIG. 5G,K) staining for INFy and TNFα, compared to control saline-treated mice and in contrast to mice treated with weekly injections of HLL, in whom no reductions were seen. Similar dichotomous patterns of staining were seen for the macrophage polarisation markers iNOS (suppl FIG. 6A-D) and CD206 (suppl FIG. 6E-H), with staining for the former suppressed in both CD68+ and CD68-negative cells, but staining of the latter enhanced in CD68+ cells, by weekly injections of PTL060.

Thus, six weeks of weekly PTL060 injections promoted a significant reduction in plaque CD68+ cells and a significant shift in their phenotype, towards a phenotype that has previously been associated with plaque regression (CCR7+, ABCA1+, IFNγ-, IL-10+, iNOS-, CD206+).

### Mechanism of Regression: Impact of Thrombin Inhibitor Tethered to the Surface of Circulating Monocytes.

As shown already, PTL060 rapidly adheres to the surface of circulating leukocytes. To investigate the impact of this leukocyte-tethered thrombin inhibitor, in isolation to that tethered by EC or platelets and erythrocytes, we adoptively transferred CD11b+ cells labelled with the fluorescent dye PKH26 into ApoE−/− mice fed a HFD from weeks 6-22, before assessing the phenotype of the labelled plaque cells by confocal immunofluorescence microscopy 48 hours later. To avoid the potential confounding influence of transfer of PTL060 from the adoptively transferred cells to vascular membranes, for these experiments we used CD11b+ cells from CD31-Hir-Tg mice, which express covalently tethered cell surface hirudin on all monocytes, and compared the impact of labelled BL/6 cells.

At the point of adoptive transfer, MIF was expressed throughout the plaques (FIG. 6A), and significant numbers of labelled cells were recruited, such that they occupied 20-25% of plaque area (FIG. 6B, C), with no difference in the numbers of BL/6 vs CD31-Hir-Tg cells recruited (FIG. 6D). All recruited cells from the Tg strain expressed hirudin (FIG. 6C). Although the plaques already contained significant numbers of Ly6G+ granulocytes, occupying 20-25% of plaque area, and although the adoptively transferred CD11b+ populations contained granulocytes, <1% of the recruited PKH26-labelled cells co-expressed Ly6G (FIG. 6G), suggesting they were mostly monocytes, and >95% of the labelled cells, from both BL/6 and CD31-Hir-Tg, expressed CCR2 (FIG. 6H-J), suggesting they were predominantly Ly6Chi monocytes. Whereas none of the recruited BL/6 cells expressed ABCA1 or CCR7, the majority of CD31-Hir-Tg cells recruited to the plaques expressed both these markers (FIG. 6K-P). In addition, whereas the majority of recruited BL/6 cells expressed IFNγ and iNOS, these were expressed by few of the recruited CD31-Hir-Tg cells (suppl FIG. 7). Instead, a significant minority of these cells expressed IL-10 and CD206, markers not expressed by labelled BL/6 cells (suppl FIG. 7). These data illustrate that CCR2+ monocytes recruited to established plaques are polarised towards a pro-inflammatory M1 phenotype, but that a membrane tethered anti-thrombin subverts this phenotype towards one that has previously been associated with plaque regression. This strongly suggests that the shift towards regression, induced by PTL060, begins immediately post-injection via the influence of cell tethered PTL060 on the phenotype of CCR2+ monocytes recruited to existing plaques.

Monocyte Recruitment and Phenotype after Systemic PTL060.

To assess whether PTL060 reduced numbers of monocytes recruited, ApoE−/− mice were fed a HFD from 6-22 weeks, before administration of weekly PTL060 or saline for 3 weeks to the age of 25 weeks. PKH2-labelled CD11b cells from BL/6 mice were administered one week after the last injection of PTL060 (by which time all PTL060 should have left the circulation (see FIG. 2), and plaques examined 48 hours later by confocal immunofluorescence microscopy. After 3 weeks treatment with PTL060, there was significant suppression of MIF expression by vessel wall cells (FIG. 7A,B), associated with a significant reduction in the number of adoptively transferred cells recruited to the plaques, such that they occupied only 2% of plaque area compared to 20% in control mice that had received saline (FIG. 7C).

As at baseline, the monocytes recruited into plaques of saline treated animals were predominantly CCR2+(FIG. 7D,F), suggesting they belonged to the Ly6Chi subset.

However, monocytes recruited to plaques in PTL060-treated mice were predominantly CCR2-neg, suggesting they were predominantly Ly6Clo monocytes (FIG. 7E,F).

Compared to cells recruited in saline treated mice, the majority of labelled cells recruited into the plaques of PTL060 mice expressed CCR7 (FIG. 7G-1) and ABCA1 (FIG. 7J-L), as well as IL-10 (suppl FIG. 8A-C) but fewer cells expressed TNFα (suppl FIG. 8D-F) or IFNγ (suppl FIG. 8G-1). Therefore, weekly systemic delivery of PTL060 suppressed vessel wall chemokine production, significantly reduced the recruitment of adoptively transferred monocytes by >90% compared to controls and promoted recruitment of CCR2-negative monocytes, which, independently of any direct binding of PTL060 to their cell surface, had the same phenotype that has already been associated with plaque regression.

A Thrombin Inhibitor on the Surface of CD11 b+ Cells is Sufficient to Induce Regression.

To assess whether tethering of PTL060 to leukocytes alone was sufficient to induce plaque regression, we fed ApoE−/− mice a HFD from 6-22 weeks, and then adoptively transferred, by weekly IV injection during weeks 23-28, CD11b+ cells, while continuing the HFD. Control mice received cells from BL/6 mice incubated, prior to transfer, with either saline or the cytotopic tail compound only. Experimental mice received BL/6 cells pre-incubated with PTL060 or, as a positive control, cells from CD31-Hir-Tg mice. All mice receiving control cells showed progression of atherosclerosis between 23-28 weeks (FIG. 8A,B, E,F,I) that was similar in degree to that seen in saline treated controls described earlier (see FIG. 4). In contrast, mice receiving PTL060-treated BL/6 cells (FIG. 8C,G,I), or cells from CD31-Hir-Tg mice (FIG. 8D, H, I) showed regression of plaque area similar in degree to mice that had been treated with systemic PTL060 (see FIG. 4). The phenotype of regressing plaques in mice given cells from CD31-Hir-Tg mice strongly resembled those in mice receiving systemic PTL060 (FIG. 8J).

Taken together with the data from the adoptive transfer of labelled cells, these data indicate that mechanistically, the impact of systemic PTL060 treatment can be reproduced entirely by isolating a thrombin inhibitor onto the surface of circulating monocytes, suggesting that inhibiting thrombin activity on only these cells is sufficient to promote regression.

### DISCUSSION

The involvement of coagulation proteases in atherosclerosis and the impact of inhibiting them has been described by multiple groups in previous studies. For instance, ApoE^{-/-} mice made deficient in HCII, a natural thrombin inhibitor¹⁷, or carrying a DNA variant resulting in defective thrombomodulin-mediated generation of activated protein C¹⁸ develop severe atheroma, indicating that in this model, endogenous regulators of thrombin act to limit disease severity. Conversely, factor (F)Xa inhibitors^{19,20} and direct thrombin inhibitors²¹⁻²⁴ prevent atheroma progression and maintain plaque stability Systemic anticoagulants can also induce regression of atherosclerosis in ApoE−/− mice. Bea et al used megalatran in 30-week-old animals and showed reduced burden of advanced atheromatous lesions associated with plaque stability²⁵. More recently, Posthuma et al²⁶ reduced atheroma burden in 22 week old animals by 25% after daily treatment for 6 weeks with clinically relevant doses of the FXa inhibitor rivaroxaban.

These data from animal models have fed into clinical practice, and the benefit of systemic anticoagulation in patients with atherosclerosis has been most recently confirmed by the COMPASS trial, which showed that addition of rivaroxaban to aspirin in patients with stable atherosclerotic cardiovascular disease led to fewer deaths, strokes and myocardial infarction²⁷. Moreover, the PAR-1 antagonist vorapaxar has also been shown to reduce the risk of myocardial infarction in patients with stable atherosclerosis²⁸. However, these benefits were associated with a significant increase in the incidence of major bleeding events: this is the biggest drawback to using systemic anticoagulants or antiplatelet drugs for non-thrombotic diseases, as their impact on haemostasis cannot be separated from their clinical efficacy.

The development of thrombalexins built upon a foundation of tethering anti-complement compounds using a generic tail based on the myristoyl-electrostatic switch ^{29,30} We have demonstrated that several versions of thrombalexin, including PTL060 effectively bind to cell membranes, maintain potent thrombin inhibitory activity, and prevent intravascular thrombosis when infused into rodent¹² or primate kidneys¹⁴ prior to transplantation. Under these circumstances, PTL060 remains detectable in tissue for several days.

In this work, we have shown that after IV injection, PTL060 inhibits secretion of vessel wall chemokines for 1 week and prevents atheroma formation but increases the risk of bleeding for only 24 hours. Therefore, the addition of the cytotopic tail uncouples the pharmacodynamics of hirulog's effects on haemostasis from its effects on atheroma formation, so that an increased bleeding tendency is seen for only 1/7^{th} of the period between doses that both prevent plaque formation and induce plaque regression. To our knowledge, this is the first demonstration of such uncoupling, and represents a significant advance in understanding the true therapeutic potential of targeting coagulation proteases to influence inflammatory disease.

Our interest in this area began with the idea that targeting anticoagulants to cell membranes would achieve high concentrations in localised environments, such as the endothelium of an organ transplant, to inhibit vascular thrombosis. We demonstrated proof of concept using transgenically expressed fusion proteins^{15, 31}, and in the process showed that inhibiting thrombin-mediated signalling through protease activated receptors on vessels inhibited local chemokine gradients, which reduced monocyte recruitment to sites of inflammation, including after transplantation, and prolonged survival³². We then went on to show that thrombin was similarly involved in chemokine gradient generation in atherosclerosis, such that expression of a tethered anticoagulant on SMC significantly reduced the development of atheroma in ApoE−/− mice¹¹. In this new work we have confirmed that expression of tethered anticoagulants on EC is equally efficacious at suppressing vessel wall chemokine expression by both EC and SMC in ApoE−/− mice and equally effective at preventing atherosclerosis as expression on SMC. Although there was some variation in the extent of atherosclerosis development by control ApoE−/− mice fed a HFD for 4-6 weeks across temporally distinct experiments, one consistent feature was that single doses of PTL060 caused significant inhibition (≥50%) of atheroma formation compared to controls. We have not investigated the mechanism by which targeted thrombin inhibition on EC influences the phenotype of underlying SMC, but the data are consistent with the known importance of EC/SMC interplay for atheroma development³³, and one possibility is that it acts via regulation of angiopoietin-2 secretion, known to be important in atherosclerosis³⁴, which we have shown to be thrombin-dependent in a separate model system³⁵. Our most important finding was that in ApoE−/− mice fed a HFD for 16 weeks prior to weekly injections of PTL060 for six weeks, atheroma burden was significantly reduced, compared not only to control mice given either saline or an equimolar dose of parental HLL, but also in comparison to baseline, indicating that PTL060 caused regression of existing disease. This was achieved without impacting plasma lipid concentrations. A similar reduction in plaque burden was seen in mice fed a normal Chow diet. There were significantly fewer CD68+ macrophages and foam cells present after 6 weeks treatment in the regressing plaques.

In assessing the mechanisms of regression, we considered the importance of inhibiting vessel wall chemokine gradients. Continuous monocyte recruitment into the vessel wall is one of the major steps in the pathogenesis of atherosclerosis, as evidenced by studies showing that simultaneous inhibition of CCL2, CX3CR1 and CCR5 near abolishes development of atheroma in ApoE−/− mice³⁶. In addition, deficiency of MIF also impairs atheroma development in LDL-R deficient mice³⁷ an inhibitory anti-MIF antibody has been shown to prevent atherosclerosis in ApoE^{-/-} mice³⁸, and our previous work illustrated that MIF secretion was important. We confirmed that a single dose of PTL060 led to prolonged suppression of vessel wall MIF (and CCL-2), and that this associated with prevention of plaque development. In addition, recruitment of labelled CD11 b+Ly6G-monocytes, adoptively transferred 1 week after the last of three doses of PTL060, was reduced by 90%, compared to that seen in control, saline-treated mice. This is consistent with the idea that suppression of vessel wall chemokine expression, interrupting the continuous cycle of monocyte recruitment, foam cell development, cell death and vessel wall inflammation might be an important contributory mechanism of how PTL060 induces plaque regression.

However, PTL060 also modulated the phenotype of recruited monocytes/macrophages. Thus, plaque cells in the regressing plaques in PTL060-treated mice had a significantly different phenotype compared to those detected in the progressing plaques in control animals, with reduced expression of pro-inflammatory IFNγ, TNFα and iNOS, and significant increases in the proportions of cells expressing CD206, IL-10, ABCA1 and CCR7.

These phenotypic characteristics have all been associated with mechanisms of regression defined in other studies. For instance after transplantation of atheromatous aorta from ApoE−/− mice into BL/6 mice³⁹⁻⁴¹, the chemokine receptor CCR7 was shown to be important for emigration of foam cells, as demonstrated by inhibiting the chemokine ligands for CCR7⁴². In another model, LDLR−/− mice treated with an antisense to miR-33 showed regression associated with upregulated ABCA1 expression in plaque macrophages and enhanced reverse cholesterol transport⁴³, in association with increased levels of circulating HDL, consistent with the known importance of ABCA1 for cholesterol loading into HDL and with the phenotype of ABCA1-deficient mice⁴⁴. Finally, the importance of polarising new monocyte recruits to the plaque towards an M2 phenotype has been recently demonstrated in the aortic transplant model, by confirming that regression is dependent on the expression of both appropriate chemokine receptors (CCR2/CX3CR1) and the transcription factor STAT6 by recipient monocytes⁴⁵.

These phenotypic changes were evident in newly recruited monocytes, but our adoptive transfer experiments suggested that CCR2+ or CCR2− monocytes were recruited at different times following PTL060 treatment. In the first experimental setting, using CD11b+ cells from CD31-Hir-Tg mice, transferred into ApoE−/− mice fed a HFD for 16 weeks without PTL060 treatment, we showed that CCR2+ monocytes were predominantly recruited, and these displayed the phenotypic traits associated with regression. In a second experimental setting, we showed that the CD11b+ cells recruited after adoptive transfer into mice already treated with 3 doses of PTL060 were predominantly CCR2− cells but also displaying the same phenotypic traits associated with regression. In this situation, labelled cells were transferred one week after the last of three doses of PTL060, into mice in which PTL060 had been cleared from the circulation, but importantly, into mice in which significant changes in plaque phenotype had already been induced. We suggest that the differential recruitment of the CCR2-(Ly6Clo) subset, known to be precursors of M2 polarised macrophages⁴⁶, is most likely due to the conditions within the plaque already established by the PTL060. We postulate that, after the first dose, the immediate uptake of PTL060 onto the surface of circulating CCR2+ monocytes, protects them from thrombin as they are recruited into established plaques, significantly skews their phenotype as they become macrophages, and this rapidly establishes the microenvironmental conditions inside the plaque that are required to initiate regression.

Although the focus of this work has been on the impact of thrombin inhibition on the vessel wall and circulating leukocytes, they also provide a potential explanation for the mechanisms through which FXa inhibitors induce regression, though these agents would also influence signalling through PAR-2. We also showed immediate uptake of PTL060 onto circulating platelets after IV injection. Since interactions between platelets and EC and between platelets and leukocytes, via CD40, have been shown to promote leukocyte recruitment and exacerbate plaque formation in this model⁴⁷, we cannot exclude the possibility that PTL060 might be modulating these interactions.

However, the data generated showing that weekly adoptive transfer of CD11b+ cells pre-treated with PTL060, or expressing a transgenic hirudin fusion protein can induce the same degree of regression as systemic PTL060, suggests that protecting plaque-recruited monocytes from the direct effects of thrombin is the key factor required for regression. Since thrombin, via protease activated receptor-1 and cullin 3-mediated degradation is known to promote post-transcriptional downregulation of ABCA1 in macrophages⁴⁸, and is also known to promote M1 polarization of microglia after intracerebral haemorrhage⁴⁹, our data is most consistent with the hypothesis that thrombin plays a hitherto unrecognised but pivotal role in determining the inflammatory phenotype of plaque macrophages and promoting plaque progression.

### References to Example 2

1. Zernecke A, Shagdarsuren E and Weber C. Chemokines in atherosclerosis: an update. Arterioscler Thromb Vase Biol. 2008; 28:1897-908.
2. Hansson G K. Inflammation, atherosclerosis, and coronary artery disease. N Engl J Med. 2005; 352:1685-95.
3. Ross R. Genetically modified mice as models of transplant atherosclerosis. Nat Med. 1996; 2:527-8.
4. Taubman M B, Fallon J T, Schecter A D, Giesen P, Mendlowitz M, Fyfe B S, Marmur J D and Nemerson Y. Tissue factor in the pathogenesis of atherosclerosis. Thromb Haemost. 1997; 78:200-4.
5. Toschi V, Gallo R, Lettino M, Fallon J T, Gertz S D, Fernandez-Ortiz A, Chesebro J H, Badimon L, Nemerson Y, Fuster V and Badimon J J. Tissue factor modulates the thrombogenicity of human atherosclerotic plaques. Circulation. 1997; 95:594-9.
6. Yamashita A, Matsuda S, Matsumoto T, Moriguchi-Goto S, Takahashi M, Sugita C, Sumi T, Imamura T, Shima M, Kitamura K and Asada Y. Thrombin generation by intimal tissue factor contributes to thrombus formation on macrophage-rich neointima but not normal intima of hyperlipidemic rabbits. Atherosclerosis. 2009; 206:418-26.
7. Weis J R, Pitas R E, Wilson B D and Rodgers G M. Oxidized low-density lipoprotein increases cultured human endothelial cell tissue factor activity and reduces protein C activation. FASEB J. 1991; 5:2459-65.
8. Lesnik P, Rouis M, Skarlatos S, Kruth H S and Chapman M J. Uptake of exogenous free cholesterol induces upregulation of tissue factor expression in human monocyte-derived macrophages. Proc Natl Acad Sci USA. 1992; 89:10370-4.
9. Cui M Z, Penn M S and Chisolm G M. Native and oxidized low density lipoprotein induction of tissue factor gene expression in smooth muscle cells is mediated by both Egr-1 and Spi. J Biol Chem. 1999; 274:32795-802.
10. Chen D, Weber M, Shiels P G, Dong R, Webster Z, McVey J H, Kemball-Cook G, Tuddenham E G, Lechler R I and Dorling A. Postinjury vascular intimal hyperplasia in mice is completely inhibited by CD34+ bone marrow-derived progenitor cells expressing membrane-tethered anticoagulant fusion proteins. J Thromb Haemost. 2006; 4:2191-8.
11. Chen D, Xia M, Hayford C, Tham el L, Semik V, Hurst S, Chen Y, Tam H H, Pan J, Wang Y, Tan X, Lan H Y, Shen H, Kakkar V V, Xu Q, McVey J H and Dorling A.

Expression of human tissue factor pathway inhibitor on vascular smooth muscle cells inhibits secretion of macrophage migration inhibitory factor and attenuates atherosclerosis in ApoE−/− mice. Circulation. 2015; 131:1350-60.
12. Karegli J, Melchionna T, Farrar C A, Greenlaw R, Smolarek D, Horsfield C, Charif R, McVey J H, Dorling A, Sacks S H and Smith R A. Thrombalexins: Cell-Localized Inhibition of Thrombin and Its Effects in a Model of High-Risk Renal Transplantation. Am J Transplant. 2017; 17:272-280.
13. Hamaoui K, Gowers S, Boutelle M, Cook T H, Hanna G, Darzi A, Smith R, Dorling A and Papalois V. Organ Pretreatment With Cytotopic Endothelial Localizing Peptides to Ameliorate Microvascular Thrombosis and Perfusion Deficits in Ex Vivo Renal Hemoreperfusion Models. Transplantation. 2016; 100:e128-e139.
14. Manook M, Kwun J, Burghuber C, Samy K, Mulvihill M, Yoon J, Xu H, MacDonald A L, Freischlag K, Curfman V, Branum E, Howell D, Farris A B, Smith R A, Sacks S, Dorling A, Mamode N and Knechtle S J. Thrombalexin: Use of a Cytotopic Anticoagulant to Reduce Thrombotic Microangiopathy in a Highly Sensitized Model of Kidney Transplantation. Am J Transplant. 2017; 17:2055-2064.
15. Chen D, Giannopoulos K, Shiels P G, Webster Z, McVey J H, Kemball-Cook G, Tuddenham E, Moore M, Lechler R and Dorling A. Inhibition of intravascular thrombosis in murine endotoxemia by targeted expression of hirudin and tissue factor pathway inhibitor analogs to activated endothelium. Blood. 2004; 104:1344-9.
16. Ignjatovic V. Thrombin clotting time. Methods Mol Biol. 2013; 992:131-8.
17. Vicente C P, He L and Tollefsen D M. Accelerated atherogenesis and neointima formation in heparin cofactor II deficient mice. Blood. 2007; 110:4261-7.
18. Borissoff J I, Otten J J, Heeneman S, Leenders P, van Oerle R, Soehnlein O, Loubele S T, Hamulyak K, Hackeng T M, Daemen M J, Degen J L, Weiler H, Esmon C T, van Ryn J, Biessen E A, Spronk H M and ten Cate H. Genetic and pharmacological modifications of thrombin formation in apolipoprotein e-deficient mice determine atherosclerosis severity and atherothrombosis onset in a neutrophil-dependent manner. PLoS One. 2013; 8:e55784.
19. Zhou Q, Bea F, Preusch M, Wang H, Isermann B, Shahzad K, Katus H A and Blessing E. Evaluation of plaque stability of advanced atherosclerotic lesions in apo E-deficient mice after treatment with the oral factor Xa inhibitor rivaroxaban. Mediators Inflamm. 2011; 2011:432080.
20. Hara T, Fukuda D, Tanaka K, Higashikuni Y, Hirata Y, Nishimoto S, Yagi S, Yamada H, Soeki T, Wakatsuki T, Shimabukuro M and Sata M. Rivaroxaban, a novel oral anticoagulant, attenuates atherosclerotic plaque progression and destabilization in ApoE-deficient mice. Atherosclerosis. 2015; 242:639-46.
21. Lee I O, Kratz M T, Schirmer S H, Baumhakel M and Bohm M. The effects of direct thrombin inhibition with dabigatran on plaque formation and endothelial function in apolipoprotein E-deficient mice. The Journal of pharmacology and experimental therapeutics. 2012; 343:253-7.
22. Kadoglou N P, Moustardas P, Katsimpoulas M, Kapelouzou A, Kostomitsopoulos N, Schafer K, Kostakis A and Liapis C D. The beneficial effects of a direct thrombin inhibitor, dabigatran etexilate, on the development and stability of atherosclerotic lesions in apolipoprotein E-deficient mice: dabigatran etexilate and atherosclerosis. Cardiovasc Drugs Ther. 2012; 26:367-74.
23. Pingel S, Tiyerili V, Mueller J, Werner N, Nickenig G and Mueller C. Thrombin inhibition by dabigatran attenuates atherosclerosis in ApoE deficient mice. Arch Med Sci. 2014; 10:154-60.
24. Preusch M R, Ieronimakis N, Wijelath E S, Cabbage S, Ricks J, Bea F, Reyes M, van Ryn J and Rosenfeld M E. Dabigatran etexilate retards the initiation and progression of atherosclerotic lesions and inhibits the expression of oncostatin M in apolipoprotein E-deficient mice. Drug Des Devel Ther. 2015; 9:5203-11.
25. Bea F, Kreuzer J, Preusch M, Schaab S, Isermann B, Rosenfeld M E, Katus H and Blessing E. Melagatran reduces advanced atherosclerotic lesion size and may promote plaque stability in apolipoprotein E-deficient mice. Arterioscler Thromb Vase Biol. 2006; 26:2787-92.
26. Posthuma J J, Posma J J N, van Oerle R, Leenders P, van Gorp R H, Jaminon A M G, Mackman N, Heitmeier S, Schurgers L J, Ten Cate H and Spronk H M H. Targeting Coagulation Factor Xa Promotes Regression of Advanced Atherosclerosis in Apolipoprotein-E Deficient Mice. Sci Rep. 2019; 9:3909.
27. Eikelboom J W, Connolly S J, Bosch J, Dagenais G R, Hart R G, Shestakovska O, Diaz R, Alings M, Lonn E M, Anand S S, Widimsky P, Hori M, Avezum A, Piegas L S, Branch K R H, Probstfield J, Bhatt D L, Zhu J, Liang Y, Maggioni A P, Lopez-Jaramillo P, O'Donnell M, Kakkar A K, Fox K A A, Parkhomenko A N, Ertl G, Stork S, Keltai M, Ryden L, Pogosova N, Dans A L, Lanas F, Commerford P J, Torp-Pedersen C, Guzik T J, Verhamme P B, Vinereanu D, Kim J H, Tonkin A M, Lewis B S, Felix C, Yusoff K, Steg P G, Metsarinne K P, Cook Bruns N, Misselwitz F, Chen E, Leong D, Yusuf S and Investigators C. Rivaroxaban with or without Aspirin in Stable Cardiovascular Disease. N Engl J Med. 2017; 377:1319-1330.
28. Kidd S K, Bonaca M P, Braunwald E, De Ferrari G M, Lewis B S, Merlini P A, Murphy S A, Scirica B M, White H D and Morrow D A. Universal Classification System Type of Incident Myocardial Infarction in Patients With Stable Atherosclerosis: Observations From Thrombin Receptor Antagonist in Secondary Prevention of Atherothrombotic Ischemic Events (TRA 2 degrees P)-TIMI 50. J Am Heart Assoc. 2016; 5.
29. Thelen M, Rosen A, Nairn A C and Aderem A. Regulation by phosphorylation of reversible association of a myristoylated protein kinase C substrate with the plasma membrane. Nature. 1991; 351:320-2.
30. Smith R A. Targeting anticomplement agents. Biochem Soc Trans. 2002; 30:1037-41.
31. Chen D, Weber M, McVey J H, Kemball-Cook G, Tuddenham E G, Lechler R I and Dorling A. Complete inhibition of acute humoral rejection using regulated expression of membrane-tethered anticoagulants on xenograft endothelium. Am J Transplant. 2004; 4:1958-63.
32. Chen D, Carpenter A, Abrahams J, Chambers R C, Lechler R I, McVey J H and Dorling A. Protease-activated receptor 1 activation is necessary for monocyte chemoattractant protein 1-dependent leukocyte recruitment in vivo. J Exp Med. 2008; 205:1739-46.
33. Li M, Qian M, Kyler K and Xu J. Endothelial-Vascular Smooth Muscle Cells Interactions in Atherosclerosis. Front Cardiovasc Med. 2018; 5:151.
34. Yu H, Moran C S, Trollope A F, Woodward L, Kinobe R, Rush C M and Golledge J. Angiopoietin-2 attenuates angiotensin II-induced aortic aneurysm and atherosclerosis in apolipoprotein E-deficient mice. Sci Rep. 2016; 6:35190.
35. Chen D, Li K, Tham E L, Wei L L, Ma N, Dodd P C, Luo Y, Kirchhofer D, McVey J H and Dorling A. Inhibition of Angiopoietin-2 Production by Myofibrocytes Inhibits Neointimal Hyperplasia After Endoluminal Injury in Mice. Frontiers in immunology. 2018; 9:1517.
36. Combadiere C, Potteaux S, Rodero M, Simon T, Pezard A, Esposito B, Merval R, Proudfoot A, Tedgui A and Mallat Z. Combined inhibition of CCL2, CX3CR1, and CCR5 abrogates Ly6C(hi) and Ly6C(lo) monocytosis and almost abolishes atherosclerosis in hypercholesterolemic mice. Circulation. 2008; 117:1649-57.
37. Pan J H, Sukhova G K, Yang J T, Wang B, Xie T, Fu H, Zhang Y, Satoskar A R, David J R, Metz C N, Bucala R, Fang K, Simon D I, Chapman H A, Libby P and Shi G P.

Macrophage migration inhibitory factor deficiency impairs atherosclerosis in low-density lipoprotein receptor-deficient mice. Circulation. 2004; 109:3149-53.
38. Burger-Kentischer A, Gobel H, Kleemann R, Zernecke A, Bucala R, Leng L, Finkelmeier D, Geiger G, Schaefer H E, Schober A, Weber C, Brunner H, Rutten H, Ihling C and Bernhagen J. Reduction of the aortic inflammatory response in spontaneous atherosclerosis by blockade of macrophage migration inhibitory factor (MIF). Atherosclerosis. 2006; 184:28-38.
39. Feig J E. Regression of atherosclerosis: insights from animal and clinical studies. Ann Glob Health. 2014; 80:13-23.
40. Fisher E A. Regression of Atherosclerosis: The Journey From the Liver to the Plaque and Back. Arterioscler Thromb Vase Biol. 2016; 36:226-35.
41. Moore K J, Sheedy F J and Fisher E A. Macrophages in atherosclerosis: a dynamic balance. Nat Rev Immunol. 2013; 13:709-21.
42. Trogan E, Feig J E, Dogan S, Rothblat G H, Angeli V, Tacke F, Randolph G J and Fisher E A. Gene expression changes in foam cells and the role of chemokine receptor CCR7 during atherosclerosis regression in ApoE-deficient mice. Proc Natl Acad Sci USA. 2006; 103:3781-6.
43. Rayner K J, Sheedy F J, Esau C C, Hussain F N, Temel R E, Parathath S, van Gils J M, Rayner A J, Chang A N, Suarez Y, Fernandez-Hernando C, Fisher E A and Moore K J. Antagonism of miR-33 in mice promotes reverse cholesterol transport and regression of atherosclerosis. J Clin Invest. 2011; 121:2921-31.
44. Aiello R J, Brees D and Francone O L. ABCA1-deficient mice: insights into the role of monocyte lipid efflux in HDL formation and inflammation. Arterioscler Thromb Vase Biol. 2003; 23:972-80.
45. Rahman K, Vengrenyuk Y, Ramsey S A, Vila N R, Girgis N M, Liu J, Gusarova V, Gromada J, Weinstock A, Moore K J, Loke P and Fisher E A. Inflammatory Ly6Chi monocytes and their conversion to M2 macrophages drive atherosclerosis regression. J Clin Invest. 2017; 127:2904-2915.
46. Woollard K J and Geissmann F. Monocytes in atherosclerosis: subsets and functions. Nat Rev Cardiol. 2010; 7:77-86.
47. Gerdes N, Seijkens T, Lievens D, Kuijpers M J, Winkels H, Projahn D, Hartwig H, Beckers L, Megens R T, Boon L, Noelle R J, Soehnlein O, Heemskerk J W, Weber C and Lutgens E. Platelet C D40 Exacerbates Atherosclerosis by Transcellular Activation of Endothelial Cells and Leukocytes. Arterioscler Thromb Vase Biol. 2016; 36:482-90.
48. Raghavan S, Singh N K, Mani A M and Rao G N. Protease-activated receptor 1 inhibits cholesterol efflux and promotes atherogenesis via cullin 3-mediated degradation of the ABCA1 transporter. J Biol Chem. 2018; 293:10574-10589.
49. Wan S, Cheng Y, Jin H, Guo D, Hua Y, Keep R F and Xi G. Microglia Activation and Polarization After Intracerebral Hemorrhage in Mice: the Role of Protease-Activated Receptor-1. Transl Stroke Res. 2016; 7:478-487.

TABLE 1
Effect of PTL060 on body mass and plasma lipids in ApoE-/- mice

| | | Aortic Tx recipients*-fed HFD 6-14 weeks | | | | | |
|---|---|---|---|---|---|---|---|
| Prevention | | | CD31- | | | CD31- | P |
| experiments | | BL/6 | TFPI-Tg | P value | BL/6 | Hlr-Tg | value† |
| Body | Age 6 weeks | 18.6 ± 0.27 | 18.6 ± 0.25 | 0.89 | 19.6 ± 0.42 | 19.6 ± 0.31 | 0.96 |
| mass | End of Exp. | 25.2 ± 1.71 | 24.6 ± 1.36 | 0.70 | 26.8 ± 0.34 | 25.9 ± 0.77 | 0.32 |

| | | | | | | |
|---|---|---|---|---|---|---|
| Cholesterol (mmol/L) | 53.1 ± 3.31 | 52.9 ± 3.67 | 0.97 | 50.1 ± 4.98 | 50.2 ± 5.77 | 0.99 |
| Triglycerides (mmol/L) | 2.1 ± 0.14 | 2.0 ± 0.20 | 0.72 | 2.0 ± 0.13 | 2.0 ± 0.14 | 0.70 |
| HDL(mmol/L) | 1.5 ± 0.11 | 1.5 ± 0.10 | 0.90 | 1.6 ± 0.07 | 1.5 ± 0.08 | 0.60 |
| LDL (mmol/L) | 57.2 ± 2.73 | 55.2 ± 3.49 | 0.46 | 63.9 ± 9.97 | 61.0+6.28 | 0.83 |

Effect of PTL060 on body mass and plasma lipids in ApoE-/- mice

| | | Single injection*-fed HFD 6-12 weeks | | | | | |
|---|---|---|---|---|---|---|---|
| Prevention | | | HLL | PTL060 | PTL060 | PTL060 | P |
| experiments | | PBS | (5 μg/g) | (2.5 μg/g) | (5 μg/g) | (10 μg/g) | value† |
| Body | Age 6 weeks | 19.0 ± 0.40 | 19.4 ± 0.22 | 19.4 ± 0.44 | 19.4 ± 0.38 | 19.2 ± 0.24 | 0.87 |
| mass | End of Exp. | 32.0 ± 1.63 | 32.0 ± 0.99 | 31.4 ± 0.84 | 29.8 ± 0.35 | 29.4 ± 0.73 | 0.36 |
| Cholesterol (mmol/L) | | 51.0 ± 5.56 | 54.1 ± 5.70 | 49.3 ± 4.56 | 50.3 ± 4.36 | 50.1 ± 5.44 | 0.97 |
| Triglycerides (mmol/L) | | 2.1 ± 0.31 | 2.1 ± 0.36 | 2.2 ± 0.28 | 2.2 ± 0.27 | 2.1 ± 0.30 | 1.00 |
| HDL(mmol/L) | | 1.4 ± 0.28 | 1.5 ± 0.08 | 1.6 ± 0.12 | 1.5 ± 0.09 | 1.5 ± 0.09 | 0.92 |
| LDL (mmol/L) | | 55.6 ± 6.64 | 54.7 ± 4.48 | 54.1 ± 4.72 | 51.7 ± 5.89 | 52.8 ± 5.42 | 0.99 |
| | | | Series 1 HFD 6-28 weeks | | | | |
| Regression | | 6-week | | | | PTL060 | P |
| experiments | | old | Baseline^{Ø} | PBS^{¥} | Tail only | (10 μg/g) | value† |
| Body | Age 6 weeks | — | 19.3 ± 0.28 | 19.6 ± 0.37 | 19.1 ± 0.15 | 19.3 ± 0.38 | 0.74 |
| mass | Age 22 weeks | — | 30.5 ± 0.58 | 32.8 ± 0.44 | 30.4 ± 0.90 | 32.0 ± 0.81 | 0.19 |
| | End of Exp. | — | — | 33.2 ± 0.31 | 31.8 ± 0.73 | 28.7 ± 1.55 | 0.03 |
| Cholesterol (mmol/L) | | 10.3 ± 4.6 | 56.7 ± 6.08 | 61.5 ± 6.51 | 61.9 ± 6.35 | 57.9 ± 3.61 | 0.88 |
| Triglycerides (mmol/L) | | 0.36 ± 0.14 | 2.08 ± 0.15 | 2.25 ± 0.11 | 2.49 ± 0.13 | 2.19 ± 0.16 | 0.26 |
| HDL(mmol/L) | | 4.46 ± 1.1 | 1.54 ± 0.007 | 1.49 ± 0.06 | 1.46 ± 0.06 | 1.56 ± 0.06 | 0.65 |
| LDL (mmol/L) | | 14.4 ± 2.8 | 46.5 ± 3.39 | 56.1 ± 2.92 | 57.4 ± 2.57 | 50.8 ± 2.84 | 0.07 |
| | | Series 2 HFD 6-28 weeks | | | | | |
| | | | | | PTL060 | PTL060 | P |
| | | Baseline^{Ø} | PBS | HLL (5 μg/g) | (5 μg/g) | (10 μg/g) | value† |
| Body | Age 6 weeks | 20.2 ± 0.39 | 20.1 ± 0.38 | 20.6 ± 0.07 | 20.1 ± 0.30 | 20.3 ± 0.26 | 0.73 |
| mass | Age 22 weeks | 31.5 ± 1.34 | 31.4 ± 0.84 | 31.1 ± 0.66 | 31.2 ± 1.00 | 31.9 ± 0.78 | 0.85 |
| | End of Exp. | — | 32.9 ± 1.19 | 31.4 ± 0.33 | 30.2 ± 0.93 | 29.8 ± 0.59 | 0.11 |

| | | | | | | |
|---|---|---|---|---|---|---|
| Cholesterol (mmol/L) | 54.9 ± 6.25 | 56.9 ± 6.93 | 54.1 ± 5.39 | 53.9 ± 6.31 | 54.3 ± 13.1 | 0.41 |
| Triglycerides (mmol/L) | 2.27 ± 0.28 | 1.68 ± 0.31 | 2.21 ± 0.38 | 2.2 ± 0.33 | 2.16 ± 0.69 | 0.86 |
| HDL(mmol/L) | 1.61 ± 0.12 | 1.58 ± 0.11 | 1.78 ± 0.2 | 1.6 ± 0.16 | 1.68 ± 0.38 | 0.27 |
| LDL (mmol/L) | 53.8 ± 3.51 | 62.7 ± 4.43 | 63.5 ± 3.55 | 60.6 ± 6.79 | 63.3 ± 16.7 | 0.97 |

HFD; high fat diet.
BL/6; C57BL/6J.
HLL; hirulog modified to accept the myristoyl tail (NB: HLL 5 μg is equimolar to PTL060 10 μg).
PBS; phosphate buffered saline.
Exp.; experiment.
HDL; high density lipoprotein.
LDL; low density lipoprotein.
*In prevention experiments, aortic transplants performed and single injections given to mice aged 8 weeks, 2 weeks after starting HFD.
^{Ø}Baseline = week 22. Mice in the ‘baseline’ groups were harvested at this timepoint prior to any treatment
^{¥}Treatments given weekly by IV injection for 6 weeks (mice aged 22-28 weeks).
†Kruskal Wallis Test for multiple groups (NB: values from 6-week old mice not included in comparisons)

### Example 3—PTL032 and PTL0GC1

Inspired by their new understanding of how PTL060 works, the inventors devised the idea that generation of a tethered PAR-1 antagonist, which would inhibit the signalling of thrombin through PAR-1 (by virtue of the PAR-1 antagonist domain), and be endowed with the uncoupled pharmacodynamic impact on haemostasis and inflammation (by virtue of the cytotopic tail) would have the same impact on atheroma regression as PTL060.

Following on from this idea, the inventors worked to generate a new cytotopic PAR-1 antagonist, called PTL032, by conjugating a known PAR-1 antagonist, 3-mercaptopropionyl-Phe-Cha-Cha-Arg-Lys-Pro-Asn-Asp-Lys-NH₂ (3-mercaptoproprionyl-F-Cha-Cha-RKPNDK amide), to the cytotopic tail via a linker.

The inventors also generated another new cytotopic PAR-1 antagonist, called PTL0GC-1, containing the same active PAR-1 element joined to the cytotopic tail via a different linker, which lacks a disulphide bond.

The structures are shown in FIG. 16.

FIG. 16 shows the structure of PTL032 and of PTL0GC-1. The primary structures comprise the active component 3-mercaptopropionyl-Phe-Cha-Cha-Arg-Lys-Pro-Asn-Asp-Lys-NH₂ linked via a disulphide bond in PTL032 or via a thioether bond in PTL0GC-1 to a peptidic membrane-localising sequence. The theoretical MW is approximately 3.35 kD.

### Manufacture

The component parts (peptides/peptidic components) are prepared by standard techniques, for example prepared in the appropriate activated form by solid phase synthesis, unless otherwise stated.

Particular embodiments of the invention may be finally prepared by conjugation, for example as described below:

### PTLOGC-1

The final conjugation conditions comprised (Myr)2KS SKSPS KKDDK KPGDK(Bromoacetate)-NH2 (1 eq) and 3-Mercaptopropionyl-Phe-Cha-Cha-Arg-Lys-Pro-Asn-Asp-Lys-NH2 (1.2 eq) at 2 mg/ml concentration, based on the (Myr)2KS SKSPS KKDDK KPGDK(Bromoacetate)-NH2 in sodium phosphate buffer (50 mM, pH 6.5). The reaction was carried out at room temperature with stirring for 18 hours. Upon completion, the conjugation reaction was filtered and purified using an AKTA HPLC system using a Phenomenex Luna C18(2) 15 μm 100 Å 250×20 mm column. For the method, mobile phase A was 0.1% v/v TFA in water and mobile phase B was 0.1% v/v TFA in acetonitrile. Flow rate was variable with UV monitoring at 220, 240 and 280 nm. The gradient was 10-68% B over 12 CV. Fractions containing the product at >95% purity were pooled where appropriate. Solutions were freeze dried in round bottomed flasks for 2 days to give a lyophilized TFA salt.

### PTL032

(Myr)2KS SKSPS KKDDK KPGDC acid was prepared in the activated S-(2-pyridyl)thiocysteine form by solid phase synthesis. The conjugation reaction between (Myr)2KS SKSPS KKDDK KPGDC(S-2-thiopyridyl)-OH (5 mg, 0.932 micromoles thiol equivalent) in DMSO (0.1 ml) and 3-Mercaptopropionyl-Phe-Cha-Cha-Arg-Lys-Pro-Asn-Asp-Lys-NH2 (1.22 mg, 0.94 micromoles) in PBS buffer (0.1 ml) was performed on ice for 3 hours, then 100 ul fractionated on a Superdex 10/300GL peptide column (GE Healthcare, Uppsala, Sweden) at 22° C. using an AKTA purifier pump system (GE Healthcare, Sweden) in 0.02 mM sodium phosphate buffer pH 7.0 run at 0.5 ml/min. The product eluted in a UV-detectable peak at 8.1 ml and a pool between 7.8 and 8.5 ml was collected and stored at −80° C.

### Example 4—Comparative Data

In a direct comparison of PTL060 with PTL032, given at equimolar doses, the inventors were surprised to find that the latter demonstrated considerably better regression of atherosclerosis than PTL060 (FIG. 17), essentially completely eliminating atherosclerotic plaques in 4 out of the 6 animals tested.

FIG. 17: Left panel shows the % cross sectional area of the aortic root occupied by atheroma, whereas right panel shows % of whole thoracic and abdominal aorta, viewed en face, occupied by atheroma, both assessed by Oil Red O staining. Graphs show individual data points from a single mouse (N=6 per group), with mean (dotted line). All were ApoE−/− mice fed a HFD for 16 weeks. ‘Baseline’ group was assessed at this point. The remaining groups then received weekly IV injections of either PTL032 (7 μg/g) or PTL060 at equimolar dose (=10 μg/g) or saline control for 6 further weeks, whilst remaining on a HFD. p values by Mann Whitney U.

As evidenced by ELISA testing of plasma, regression of the atheroma in the mice given PTL032 was associated with suppression of important chemokines and cytokines, almost back to levels seen in very young mice prior to the development of atherosclerosis (FIG. 18).

FIG. 18: Illustrating the systemic proinflammatory state that accompanies development of atherosclerosis in ApoE^{-/-} mice fed a high fat diet, and the impact of PTL032 on that inflammation, compared to PTL060 and controls. Cytokines (interferon-gamma TNFalpha) and chemokines (macrophage migration inhibitory factor, MIF and CCL2) measured by ELISA of plasma samples harvested from mice at the times indicated. HFD=high fat diet. 6/52=6 week old mice, prior to feeding the high fat diet. 16/52=16 weeks. 22/52=22 weeks. PTL060 given at a dose of 10 μg/g weekly for 6/52 by IV injection. Bivalirudin and PTL032 administered at equimolar doses to PTL060 using same dosing schedule. PTL032 inhibits the systemic pro-inflammatory response, almost back to that seen in 6-week old mice, prior to development of atherosclerosis.

All these data provide evidence that PTL032 induces significant regression of established disease in this model of atherosclerosis, with an efficiency better than the tethered direct thrombin inhibitor PTL060 in these direct comparative experiments.

### Example 5—Further Experimental Support

Experimental evidence to explain the remarkable unexpected potency of PTL032/PTL0GC-1 is provided.

Firstly we show how 3-mercaptopropionyl-Phe-Cha-Cha-Arg-Lys-Pro-Asn-Asp-Lys-NH₂ is different to other PAR-1 antagonists.

This example uses a model of contact hypersensitivity. Here, mice are challenged on the abdomen with the contact sensitizer oxazolone, before a re-challenge 4 days later on both sides of the ear with oxazolone (on the right) or vehicle alone (on the left).

Swelling of the ears, measured with a micrometer, reflects the influx of monocytes/macrophages and T cells in a typical delayed-type hypersensitivity (DTH) response. Dual inhibition of both PAR-1 and PAR-2 signalling in mice expressing the tissue factor inhibitor was associated with exacerbated responses to re-challenge with oxazolone (i.e. increased ear swelling), whereas in the mice expressing hirudin, in which PAR-2 signalling was allowed, ear swelling was significantly prevented, even compared to negative littermate controls.

We have followed up these early observations with further experiments using selective PAR agonists and antagonists administered to WT mice simultaneously with the second exposure to oxazolone. As illustrated in FIG. 19, ear swelling of WT mice is inhibited by a PAR-1 antagonist and exacerbated by a PAR-1 agonist.

FIG. 19: Model of DTH involving contact sensitivity to oxazolone. WT mice were administered a PAR-1 agonist or antagonist at the point of second exposure to oxazolone, before ear thickness was assessed 24 hours later, and mice sacrificed for immunohistological analysis of cross sections of the affected ears, to determine the proportional area occupied by CD68+ cells (macrophages). *p<0.05. **p<0.01. ***p<0.001

The swelling directly associates with the proportion of CD68+ cells (macrophages) that can be found infiltrating the ears.

However, the effects of PAR-2 specific reagents are exactly opposite: a PAR-2 antagonist exacerbates ear swelling whereas a PAR-2 agonist inhibits it (FIG. 20). FIG. 20: Model of DTH involving contact sensitivity to oxazolone. WT mice were administered a PAR-2 agonist or antagonist at the point of second exposure to oxazolone, before ear thickness was assessed 24 hours later, and mice sacrificed for immunohistological analysis of cross sections of the affected ears, to determine the proportional area occupied by CD68+ cells (macrophages). *p<0.05. **p<0.01. ***p<0.001. ****p<0.0001

Finally, in the same model, we have directly compared the impact of 3-mercaptoproprionyl-F-Cha-Cha-RKPNDK amide to the other PAR reagents. As can be seen in FIG. 21, it inhibits ear swelling and infiltration by CD68+ cells better than when either a pure PAR-1 antagonist or PAR-2 agonist are administered separately, and even inhibits CD68 infiltration better than when both reagents are administered together.

FIG. 21: Model of DTH involving contact sensitivity to oxazolone. Showing the impact of 3-mercaptoproprionyl-F-Cha-Cha-RKPNDK amide on ear swelling and infiltration of CD68+ cells on WT mice in comparison to pure PAR-1 and PAR-2 reagents administered singly or in combination. *p<0.05. **p<0.01. ***p<0.001. ****p<0.0001

We have also studied the impact of 3-mercaptoproprionyl-F-Cha-Cha-RKPNDK amide on the chemokine expression by both SMC and EC, relevant as influencing chemokine expression at the vessel wall is one of the mechanisms by which PTL060 induces regression (see earlier example). In this model, cells are purified from a WT mouse and grown in vitro as previously described (Chen et al 2015 Circulation vol 131 pages 1350-1360. Once confluent, cells are incubated with PAR agonists/antagonists for 12 hours, followed by, where appropriate, a further 1 hour incubation with thrombin. The chemokines secreted over the following 48 hours are measured in the supernatant by ELISA. As shown in FIG. 22, secretion of MIF (and CCL-2), chemokines involved in recruitment of Ly6Chi (CCR2+) monocytes is stimulated by PAR-1 agonists and less so by a PAR-2 agonist and 3-mercaptoproprionyl-F-Cha-Cha-RKPNDK amide. FIG. 22: Secretion of MIF and CX3CL1 by SMC after 12 hour stimulation with either a pure PAR-1 agonist, pure PAR-2 agonist, or by 3-mercaptoproprionyl(MP)—F-Cha-Cha-RKPNDK amide, followed by 48 hour culture period. All used at 10 mM. Patterns of secretion of CCL-2 resemble those of MIF, whereas secretion of CCL5 resembles CX3CL1.

In contrast, the chemokines CX3CL1 (and CCL5), both of which promote recruitment of Ly6Clo (CCR2-) monocytes, is stimulated better by a PAR-2 agonist and 3-mercaptoproprionyl-F-Cha-Cha-RKPNDK amide than by a PAR-1 agonist.

These differences in PAR-1-dependent and PAR-2 dependent chemokine secretion impact significantly when thrombin-mediated secretion is antagonised by either a pure PAR-1 antagonist or 3-mercaptoproprionyl-F-Cha-Cha-RKPNDK amide. As shown in FIG. 23, thrombin-mediated MIF (and CCL-2) secretion is inhibited equally by both a pure PAR-1 antagonist and 3-mercaptoproprionyl-F-Cha-Cha-RKPNDK amide. However, thrombin-mediated CX3CL1 secretion is inhibited only by the pure PAR-1 antagonist. In contrast, CX3CR1 secretion is maintained by 3-mercaptoproprionyl-F-Cha-Cha-RKPNDK amide, primarily because of the provision of PAR-2 stimulation by the latter. Thus 3-mercaptoproprionyl-F-Cha-Cha-RKPNDK amide promotes differential inhibition of MIF and CCL-2, chemokines involved in recruitment of Ly6Chi (CCR2+) monocytes whilst promoting secretion of CX3CL1 and CCL5, chemokines involved in recruitment of Ly6Clo (CCR2−) monocytes.

FIG. 23: Secretion of MIF and CX3CL1 by SMC after 12 hour stimulation with either a pure PAR-1 antagonist, pure PAR-2 agonist, or 3-mercaptoproprionyl-F-Cha-Cha-RKPNDK amide, all used at 10 mM, followed by a 1 hour incubation with 10 mM thrombin. Chemokines measured 48 hours later. Secretion of CCL-2 resembles that of MIF, whereas secretion of CCL5 resembles CX3CL1.

In summary the inventors have performed the work above to illustrate the differences between the parental compound (3-mercaptoproprionyl-F-Cha-Cha-RKPNDK amide) and other pure PAR-1 antagonists.

The inventors show a comparative experiment with PTL032, indicating that in a formal ‘head to head’ test it is more potent than PTL060.

The inventors have performed multiple experiments in vivo with PTL060, which serve to exemplify how cytotopic proteins with the same membrane binding compound behave in vivo after IV injection.

It is being confirmed that PTL0GC-1 causes significant regression of atherosclerosis in ApoE−/− mice and is more effective than equimolar doses of either PTL060 or a pure PAR-1 antagonist.

It is being confirmed that PTL0GC-1 inhibits development of renal fibrosis in a model of aristolochic acid nephropathy (AAN) and is more effective than equimolar doses of either PTL060 or a pure PAR-1 antagonist.

Studies in a large animal model (pig), formal toxicology studies, and generation of GMP-grade material for clinical use follow.

### Example 6: Further Experimental Support

Research published after the earliest priority date includes (Chen D, Li K, Festenstein S, et al. Regression of Atherosclerosis in ApoE−/− Mice Via Modulation of Monocyte Recruitment and Phenotype, Induced by Weekly Dosing of a Novel “Cytotopic” Anti-Thrombin Without Prolonged Anticoagulation. J Am Heart Assoc 2020; 9:e014811; and Wilkinson H, Leonard H, Chen D, et al. PAR-1 signalling on macrophages is required for effective in vivo delayed type hypersensitivity responses. iScience 2021; 21). These papers together set out:

a) The beneficial impact of PTL060 on both atherosclerosis and delayed type hypersensitivity (DTH);
b) PTL060 operates via two mechanisms in atherosclerosis.
i) A switch from recruitment of Ly6Chi (CCR2-pos) monocytes into atherosclerotic plaques, to recruitment of Ly6Clo (CCR2-neg) monocytes that express a ‘regression phenotype’ (CD206+, ABCA1+, IL-10+, CCR7+, iNOS−).
ii) A direct effect of PTL060 on Ly6Chi monocytes causing expression of a ‘regression phenotype’ (CD206+, ABCA1+, IL-10+, CCR7+, iNOS−);
c) Of these, the second mechanism appears dominant, as evidenced by adoptive transfer of PTL060 pre-treated monocytes, which induces the same degree of regression associated with IV treatment of PTL060;
d) The direct effect of thrombin on monocytes is explained by PAR-1 signals mediating downregulation of the reverse cholesterol transporter ABCA1, resulting in increased membrane expression of cholesterol rich microdomains, and movement of the receptor for interferon gamma (IFNg) into these areas. This enhances the sensitivity of thrombin-treated cells to IFNg by 1000×, such that monocytes are polarised away from the ‘regression phenotype’ by very small concentrations of IFNg. PTL060 inhibits these mechanisms.

In this example we further show:
Compared to PTL060, equimolar PTL0GC-1 injected IV is better at inducing atheroma regression (FIG. 26) and at inhibiting the DTH response to a dermal contact sensitiser (FIG. 27)
In both these models, the impact of PTL0GC-1 is greater than exposure to an equimolar amount of its parental compound, 3-mercaptopropionyl-F-Cha-Cha-RKPNDK (3-MP).
PTL0GC-1 administered via intraperitoneal or subcutaneous routes can inhibit the DTH response (FIG. 27)
In a model of acute kidney injury due to ischaemia reperfusion injury, IV PTL0GC-1 results in quicker recovery of kidney function and body weight, and is associated with the development of less cortical fibrosis compared to control animals given saline (FIG. 28).
Experiments in the DTH model (FIG. 29, FIG. 30) and in vitro using primary smooth muscle or endothelial cells (FIG. 31, FIG. 32) confirm that the biological effect of 3-MP and PTL0GC-1 is due to its combined effect of inhibiting signalling through PAR-1, whilst at the same time delivering an agonist signal through PAR-2.
As in atherosclerosis, in the model of DTH, PTL0GC-1 results in reduced macrophage infiltration into the skin, and those macrophages present have a distinctly different phenotype to those present in controls (FIG. 30), similar to the regression phenotype in atherosclerosis.
When used in combination with thrombin, 3-MP (and by extension PTL0GC-1), inhibits secretion of chemokines associated with recruitment of Ly6Chi monocytes, but maintains those associated with recruitment of Ly6Clo monocytes, compared to a pure PAR-1 antagonist, which reduces secretion of multiple chemokines, including those associated with recruitment of Ly6Clo monocytes (FIG. 32). Without wishing to be bound by theory, this is likely one explanation for the superior effect of PTL0GC-1 compared to PTL060 in atherosclerosis.
By virtue of its PAR-1 antagonistic activity, 3-MP inhibits the direct effects of thrombin on macrophages in vitro, and thus dampens the sensitivity of macrophages to IFNγ (FIG. 33). However, 3-MP is more potent than a pure PAR-1 antagonist (FIG. 33), because of its inherent PAR-2 agonist properties. In the presence of thrombin, it is also more potent than a pure PAR-2 agonist, which also blunts the sensitivity of macrophages to IFNγ induced by thrombin (FIG. 33). This is by virtue of its PAR-1 antagonist properties.
This additional impact of 3-MP (by virtue of its inherent PAR-2 agonist activity) is not mediated via ABCA1 (FIG. 34). Instead, using an siRNA approach, the data suggest it to be mediated by the upregulation of SOCS3, a protein involved in modulating signal transduction through IFNγ (FIG. 34). Since the baseline sensitivity of untreated macrophages to IFNγ is inhibited by 3-MP (FIG. 33), this suggests that PAR-1 and PAR-2 function in vivo to determine the exquisite sensitivity of myeloid cells to endogenous inflammatory signals such as IFNγ. Thus, inhibiting PAR-1 and signalling through PAR-2 influence two separate pathways that modulate macrophage sensitivity to IFNγ.
Finally, adoptively transferred monocytes, pre-treated with PTL0GC-1 or PTL060 and injected weekly into ApoE−/− mice fed a high fat diet cause regression of atherosclerosis (FIG. 35). Pre-treatment with PTL0GC-1 induces superior regression compared to pre-treatment with PTL060, consistent with the data above.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

### SEQUENCE LISTING

SEQ ID NO: 1
CH2-CH2-F-Cha-Cha-RKPNDK-NH2
(PTLo32 and/or PTLoGC1)
SEQ ID NO: 2
[Myr2]-KSSKSPSKKDDKKPGD
(PTLo32-including Cysteine residue)
SEQ ID NO: 3
[Myr2]-KSSKSPSKKDDKKPGDC
(PTLoGC1-including Lysine residue)
SEQ ID NO: 4
[Myr2]-KSSKSPSKKDDKKPGDK

1. A compound comprising:
CH2-CH2-F-Cha-Cha-RKPNDK-NH2
joined via a linking group to
[Myr2]-KSSKSPSKKDDKKPGD.

2. A compound according to claim 1 which has the formula: 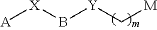
wherein
A is CH2-CH2-F-Cha-Cha-RKPNDK-NH2
M is [Myr2]-KSSKSPSKKDDKKPGD
X is S, O, or NR
B is optional and is an optionally substituted C1 to C6 alkyl
Y is S, O, or NR
m is 1, 2, 3, 4, 5, or 6
wherein each R is independently selected from H, or optionally substituted C1-6 alkyl

3. A compound according to claim 1 or claim 2 wherein said linking group comprises a disulphide bridge.

4. A compound according to claim 1 or claim 2 wherein said linking group consists of a cysteine residue-disulphide bridge.

5. A compound according to claim 1 or claim 2 wherein said linking group comprises a thioether group.

6. A compound according to claim 1 or claim 2 wherein said linking group consists of a lysine residue-thioether group.

7. A compound according to any of claims 1 to 4 which has the formula:

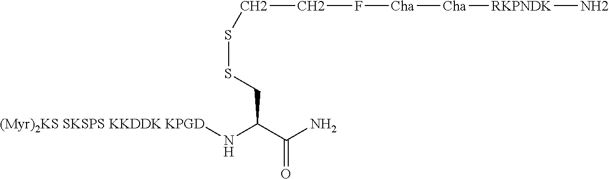

8. A compound according to any of claims 1, 2, 5 or 6 which has the formula:

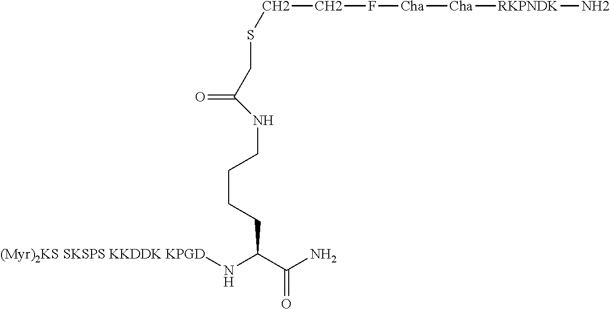

9. A compound according to any of claims 1 to 8 for use in medicine.

10. A compound according to any of claims 1 to 8 for use as a medicament.

11. Use of a compound according to any of claims 1 to 8 for the manufacture of a medicament for atheroma.

12. Use of a compound according to any of claims 1 to 8 for the manufacture of a medicament for atherosclerosis.

13. Use of a compound according to any of claims 1 to 8 for the manufacture of a medicament for inducing regression of atherosclerosis.

14. A compound according to any of claims 1 to 8 for use in treatment of atheroma.

15. A compound according to any of claims 1 to 8 for use in treatment of atherosclerosis.

16. A compound according to any of claims 1 to 8 for use in inducing regression of atherosclerosis.

17. A method of treatment comprising administering a therapeutic amount of a compound according to any of claims 1 to 8 to a subject in need of same.

18. A method of treating atheroma in a subject comprising administering a therapeutic amount of a compound according to any of claims 1 to 8 to said subject.

19. A method of treating atherosclerosis in a subject comprising administering a therapeutic amount of a compound according to any of claims 1 to 8 to said subject.

20. A method of inducing regression of atherosclerosis in a subject comprising administering a therapeutic amount of a compound according to any of claims 1 to 8 to said subject.

21. A method of treating or preventing Acute Kidney Injury (AKI) in a subject comprising administering a therapeutic amount of a compound according to any of claims 1 to 8 to said subject.

22. Use of a compound according to any of claims 1 to 8 for the manufacture of a medicament for treatment or prevention of Acute Kidney Injury (AKI).

23. A compound according to any of claims 1 to 8 for use in treatment or prevention of Acute Kidney Injury (AKI).

24. A method of treating or preventing delayed type hypersensitivity (DTH) in a subject comprising administering a therapeutic amount of a compound according to any of claims 1 to 8 to said subject.

25. Use of a compound according to any of claims 1 to 8 for the manufacture of a medicament for treatment or prevention of delayed type hypersensitivity (DTH).

26. A compound according to any of claims 1 to 8 for use in treatment or prevention of delayed type hypersensitivity (DTH).


SEQUENCE LISTING (4 sequences)

SEQ ID NO: 1 - protein, 9 aa, Artificial Sequence
  Synthetic Compound
  X2 (2)..(2): cyclohexylalanine (Cha)
  X3 (3)..(3): cyclohexylalanine (Cha)
FXXRKPNDK

SEQ ID NO: 2 - protein, 16 aa, Artificial Sequence
  Synthetic compound (PTL032 and/or PTL0GC1)
KSSKSPSKKDDKKPGD

SEQ ID NO: 3 - protein, 17 aa, Artificial Sequence
  Synthetic compound (PTL032 - including Cysteine residue)
KSSKSPSKKDDKKPGDC

SEQ ID NO: 4 - protein, 17 aa, Artificial Sequence
  Synthetic compound (PTL0GC1 - including Lysine residue)
KSSKSPSKKDDKKPGDK